SEQUENCE LISTING (18 sequences)

SEQ ID NO: 1 - protein, 18 aa, Artificial Sequence
  Description of Artificial Sequence Synthetic peptide
VDTRYFDDEFTAQSITIT

SEQ ID NO: 2 - protein, 17 aa, Artificial Sequence
  Description of Artificial Sequence Synthetic peptide
GDTSNFDDYEEEEIRVZ

SEQ ID NO: 3 - protein, 17 aa, Artificial Sequence
  Description of Artificial Sequence Synthetic peptide
TSTGSFMDIASTNTSNK

SEQ ID NO: 4 - protein, 18 aa, Artificial Sequence
  Description of Artificial Sequence Synthetic peptide
EPYVDFAEFYRLWSVDHG

SEQ ID NO: 5 - protein, 27 aa, Artificial Sequence
  Description of Artificial Sequence Synthetic peptide
MVAGVSEYELPEDPRWELPRDRLVLGK

SEQ ID NO: 6 - protein, 33 aa, Artificial Sequence
  Description of Artificial Sequence Synthetic peptide
MDPDELPLDEHCERLPYDASKWEFPRDRLKLGK

SEQ ID NO: 7 - protein, 31 aa, Artificial Sequence
  Description of Artificial Sequence Synthetic peptide
MKKHHHHHHGKNNPDPTIYPVLDWNDIKFQD

SEQ ID NO: 8 - protein, 10 aa, Artificial Sequence
  Description of Artificial Sequence Synthetic peptide
IDPKDLTFLK

SEQ ID NO: 9 - protein, 27 aa, Artificial Sequence
  Description of Artificial Sequence Synthetic peptide
MGGSVAAQDEFYRSGWALNMKELKLLQ

SEQ ID NO: 10 - protein, 21 aa, Artificial Sequence
  Description of Artificial Sequence Synthetic peptide
GAMGSSTRDYEIQRERIELGR

SEQ ID NO: 11 - protein, 40 aa, Artificial Sequence
  Description of Artificial Sequence Synthetic peptide
MSTASAASSSSSSSAGEMIEAPSQVLNFEEIDYKEIEVEE

SEQ ID NO: 12 - protein, 18 aa, Artificial Sequence
  Description of Artificial Sequence Synthetic peptide
GAMGSKRQWALEDFEIGR

SEQ ID NO: 13 - protein, 7 aa, Artificial Sequence
  Description of Artificial Sequence Synthetic peptide
EFGGGGG

SEQ ID NO: 14 - protein, 152 aa, Artificial Sequence
  Description of Artificial Sequence Synthetic polypeptide
SLHMIDYKEIEVEEVVGRGAFGVVCKAKWRAKDVAIKQIESESERKAFIVELRQLSRVNHPNIVKLYGACLNPVCLVMEYAEGGSLYNVLHGAEPLPYYTAAHAMSWCLQCSQGVAYLHSMQPKALIHRDLKPPNLLLVAGGTVLKICDFGT

SEQ ID NO: 15 - protein, 113 aa, Artificial Sequence
  Description of Artificial Sequence Synthetic polypeptide
GSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQ

SEQ ID NO: 16 - protein, 30 aa, Artificial Sequence
  Description of Artificial Sequence Synthetic peptide
HSLPPGEDGRVEPYVDFAEFYRLWSVDHGE

SEQ ID NO: 17 - protein, 152 aa, Artificial Sequence; identical to SEQ ID NO: 14
  Description of Artificial Sequence Synthetic polypeptide

SEQ ID NO: 18 - protein, 143 aa, Artificial Sequence
  Description of Artificial Sequence Synthetic polypeptide
GSAAWMAPEVFEGSNYSEKCDVFSWGIILWEVITRRKPFDEIGGPAFRIMWAVHNGTRPPLIKNLPKPIESLMTRCWSKDPSQRPSMEEIVKIMTHLMRYFPGADEPLQYPCQHSLPPGEDGRVEPYVDFAEFYRLWSVDHGE

(19) United States
(12) Patent Application Publication
(10) Pub. No.: US 2011/0171714 A1
(43) Pub. Date: Jul. 14, 2011

(54) CRYSTAL STRUCTURE OF TAK1-TAB1

(75) Inventors: Graham Cheetham, Abingdon (GB); Kieron Brown, Abingdon (GB); Sarah Vial, Abingdon (GB); Neesha Dedi, Abingdon (GB)

(73) Assignee: VERTEX PHARMACEUTICALS INCORPORATED, Cambridge, MA (US)

(21) Appl. No.: 12/986,404

(22) Filed: Jan. 7, 2011

Related U.S. Application Data
(63) Continuation of application No. 11/361,029, filed on Feb. 23, 2006, now abandoned.
(60) Provisional application No. 60/655,606, filed on Feb. 23, 2005.

(51) Int. Cl. C12N 9/96; C12N 9/12; G06G 7/58; G06F 19/00

(52) U.S. Cl. 435/188; 435/194; 703/11; 702/19

(57) ABSTRACT

The invention relates to molecules or molecular complexes which comprise binding pockets of TAK1 or its structural homologues. The invention relates to crystallizable compositions and crystals comprising TAK1. The present invention also relates to a data storage medium encoded with the structural coordinates of molecules and molecular complexes which comprise the TAK1 or TAK1-like ATP-binding pockets. The present invention also relates to a computer comprising such data storage material. The computer may generate a three-dimensional structure or graphical three-dimensional representation of such molecules or molecular complexes. This invention also relates to methods of using the structure coordinates to solve the structure of homologous proteins or protein complexes. In addition, this invention relates to methods of using the structure coordinates to screen for and design compounds, including inhibitory compounds, that bind to TAK1 or homologues thereof.

### CROSS-REFERENCE TO RELATED APPLICATIONS

This is a continuation application of U.S. patent application Ser. No. 11/361,029, filed Feb. 23, 2006 which claims priority to U.S. Provisional Application No. 60/655,606, filed Feb. 23, 2005. The entire contents of these applications are incorporated herein by reference.

### SEQUENCE LISTING

This application includes a Sequence Listing, as set forth in an ASCII-compliant text file named “20080121 Sequence listing txt file VPI05109.txt”, created on Dec. 30, 2008, and containing 11,079 bytes.

### TECHNICAL FIELD OF THE INVENTION

The present invention relates to the design of crystallisable transforming growth factor-beta-activated kinase 1 (TAK1) and TAK1 binding protein (TAB1) complexes and the X-ray analysis of crystalline molecules or molecular complexes of this TAK1-TAB1 chimera. The present invention provides a chimera of TAK1 and a region of the TAK1 activating domain of its protein activator TAB1. The present invention also provides for the first time the crystal structure of a TAK1-TAB1 chimera protein bound to adenosine and TAK1-TAB1 bound to a potent ATP-competitive inhibitor. The present invention also provides crystalline molecules or molecular complexes that comprise binding pockets of TAK1 kinase (TAK1) and/or its structural homologues, the structure of these molecules or molecular complexes. The present invention further provides crystals of TAK1-TAB1 complexed with adenosine and methods for producing these crystals. This invention also relates to a general strategy for the design of crystallisable protein kinases based on both sequence and structural alignments of related protein kinases with close homology that have previously been crystallized in the literature. This invention also relates to crystallizable compositions from which the protein-ligand complexes may be obtained. The present invention also relates to a data storage medium encoded with the structural coordinates of molecules and molecular complexes that comprise the ATP-binding pockets and TAB1-binding pockets of TAK1 or their structural homologues. The present invention also relates to a computer comprising such data storage material. The computer may generate a three-dimensional structure or graphical three-dimensional representation of such molecules or molecular complexes. This invention also relates to methods of using the structure coordinates to solve the structure of homologous proteins or protein complexes. This invention also relates to computational methods of using structure coordinates of the TAK1 complex(es) to screen for and design compounds, including inhibitory compounds and antibodies, that interact with TAK1, TAB1 or homologues thereof.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. (See, Hardie, G. and Hanks, S. The Protein Kinase Facts Book, I and II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J., 9:576-596 (1995); Knighton et al., Science, 253:407-414 (1991); Hiles et al., Cell, 70:419-429 (1992); Kunz et al., Cell, 73:585-596 (1993); Garcia-Bustos et al., EMBO J., 13:2352-2361 (1994)).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there has been a substantial effort in medicinal chemistry to find protein kinase inhibitors that are effective as therapeutic agents.

Among medically important serine/threonine kinases is the family of mitogen-activated protein kinases (MAPKs), which have been shown to function in a wide variety of biological processes (Davis D. J. Trends in Biochem Sci. 19 470-473 (1994); Su B. & Karin M Curr. Opin. Immunol 8 402-411 (1996); Treisman R. Curr. Opin. Cell Biol. 8 205-215 (1996)). MAPKs are activated by phosphorylation on specific tyrosine and threonine residues by MAPK kinases (MAPKKs), which are in turn activated by phosphorylation on serine and serine/threonine residues by MAPKK kinases (MAPKKKs). The MAPKKK family comprises several members including MEKK1, MEKK3, NIK and ASK1 and Raf. Different mechanisms are involved in the activation of MAPKKKs in response to a variety of extracellular stimuli including cytokines, growth factors and environmental stresses (refs).

Transforming growth factor-β (TGF-β-activated kinase 1 (TAK1) is a member of the mitogen-activated protein kinase kinase kinase (MAPKKK) family and has been shown to play critical roles in signaling pathways stimulated by transforming growth factor-β, interleukin-1 (IL-1), tumor necrosis factor-α (TNF-α), lipopolysaccharide, receptor activator of NF-κB ligand where it regulates osteoclast differentiation and activation, and IL-8 (Yamaguchi K et al. Science 270 2008-11 (1995); Ninomiya-Tsuji J et al. Nature 398 252-256 (1999); Sakurai H. et al. J. Biol. Chem 274 10641-10648 (1999); Irie T. et al. FEBS Lett. 467 160-164 (2000); Lee J. et al. J. Leukoc Biol. 68 909-915 (2000); Mizukami J et al. Mol. Cell. Biol. 22 992-1000 (2002); Wald D. et al. J. Immunol 31 3747-3754 (2002)). TAK1 regulates both the c-Jun N-terminal kinase (JNK) and p38 MAPK cascades in which it phosphorylates MAPK kinases MKK4 and MKK3/6, respectively (Wang W. et al. J. Biol. Chem. 272 22771-22775 (1997); Moriguchi T. et al. J. Biol. Chem. 271 13675-13679 (1996)). NF-kB factors regulate expression of a variety of genes involved in apoptosis, cell cycle, transformation, immune response, and cell adhesion (Barkett M and Gilmore T D. Oncogene, 18, 6910-6924 (1999). TAK1 regulates the IKB kinase (IKK) signaling pathways, leading to the activation of transcription factors AP-1 and NF-κB (Ninomiya-Tsuji J et al. Nature 398 252-256 (1999); Sakurai H. et al. J. Biol. Chem 274 10641-10648 (1999); Takaesu G. et al. J. Mol. Biol 326 110-115 (2003)). In early embryos of the amphibian Xenopus, TAK1 also participates in mesoderm induction and patterning mediated by bone morphogenetic protein (BMP), which is another transforming growth factor β family ligand (Shibuya H. et al. EMBO J. 17 1019-1028 (1998)). In addition, TAK1 is a negative regulator of the Wnt signaling pathway, in which TAK1 down-regulates transcription regulation mediated by a complex of β-catenin and T-cell factor/lymphoid enhancer factor (Meneghini M. D. et al. Nature 399 793-797 (1999); Ishitani T. et al. Nature 399 798-802 (1999)). The role of TAK1 in TNF-α and IL-1β-induced signaling events is evident from TAK1 RNAi experiments in mammalian cells (Takaesu G. et al. J. Mol. Biol. 326 105-115 (2003)) in which IL-1 and TNF-α induced NF-κB and MAPK activation were both inhibited. Over-expression of kinase dead TAK1 inhibits IL-1 and TNK-induced activation of both JNK/p38 and NF-kB (Ninomiya-Tsuji J et al. Nature 398 252-256 (1999); Sakurai H. et al. J. Biol. Chem 274 10641-10648 (1999)). TAK1-/- mouse embryonic fibroblasts have diminished IL-1-induced signaling and are embryonic lethal (E11.5) (S. Akira, personal communication). In adult mouse, TAK1 is activated in the myocardium after pressure overload. Expression of constitutively-active TAK1 in myocardium induced myocardial hypertrophy and heart failure in transgenic mice (Zhang D. et al. Nature Med. 6 556-563 (2000)).

TAK1 is activated by the TAK1 binding protein (TAB1) (Shibuya H et al. Science 272 1179-1182 (1996)) via an association with the N-terminal kinase domain of TAK1. It has been reported that the C-terminal 68 amino acids of TAB 1 is sufficient for the association and activation of TAK1 (Shibuya H et al. Science 272 1179-1182 (1996)). However, more recent work indicates that the minimum TAB 1 segment required includes only residues 480-495 (Ono K. et al. J. Biol. Chem. 276 24396-24400 (2001); Sakurai H. et al. FEBS Lett 474 141-145 (2000)). Deletion mutants of TAB1 show that the aromatic Phe484 residue is critical for TAK1 binding (Ono K. et al. J. Biol. Chem. 276 24396-24400 (2001)). Autophosphorylation of threonine/serine residues in the kinase activation loop are necessary for TAB1-induced TAK1 activation (Sakurai H. et al. FEBS Lett 474 141-145 (2000); Kishimoto K. et al. J. Biol. Chem. 275 7359-7364 (2000)), Ser192 appears as the most likely candidate since a Ser192Ala mutation shows no kinase activity (Kishimoto K. et al. J. Biol. Chem. 275 7359-7364 (2000)).

Since TAK1 is a key molecule in the pro-inflammatory NF-KB signaling pathway a TAK1 inhibitor would be effective in diseases associated with inflammation and tissue destruction such as rheumatoid arthritis and inflammatory bowel disease (Crohn's), as well as in cellular processes such as stress responses, apoptosis, proliferation and differentiation. Various pro-inflammatory cytokines and endotoxins trigger the kinase activity of endogenous TAK1 (Ninomiya-Tsuji J et al. Nature 398 252-256 (1999); Irie T et al. FEBS Lett. 467 160-164 (2000); Sakurai H. et al. J. Biol. Chem. 274 10641-10648 (1999)) and the Drosophila homolog of TAK1 was recently identified as an essential molecule for host defense signaling in Drosophila (Vidal S. et al. Genes Dev. 15 1900-1912 (1999)). A natural inhibitor of TAK1, 5Z-7-oxozeaenol, has been identified with an IC50 value of 8nM. 5Z-7-oxozeaenol has been shown to be selective for TAK1 within the MAPKKK family and relieves inflammation in a picryl chloride-induced ear swelling mouse model (Ninomiya-Tsuji J. et al. J. Biol. Chem. 278 18485 (2003)).

Accordingly, there has been an interest in finding selective inhibitors of TAK1 that are effective as therapeutic agents. A challenge has been to find protein kinase inhibitors that act in a selective manner, targeting only TAK1. Since there are numerous protein kinases that are involved in a variety of cellular responses, non-selective inhibitors may lead to unwanted side effects. In this regard, the three-dimensional structure of the kinase would assist in the rational design of inhibitors. The determination of the amino acid residues in TAK1 binding pockets and the determination of the shape of those binding pockets would allow one to design selective inhibitors that bind favorably to this class of enzymes. The determination of the amino acid residues in TAK1 binding pockets and the determination of the shape of those binding pockets would also allow one to determine the binding of compounds to the binding pockets and to, e.g., design inhibitors that can bind to TAK1.

For example, a general approach to designing inhibitors that are selective for an enzyme target is to determine how a putative inhibitor interacts with the three dimensional structure of the enzyme. For this reason it is useful to obtain the enzyme protein in crystal form and perform X-ray diffraction techniques to determine its three dimensional structure coordinates. If the enzyme is crystallized as a complex with a ligand, one can determine both the shape of the enzyme binding pocket when bound to the ligand, as well as the amino acid residues that are capable of close contact with the ligand. By knowing the shape and amino acid residues in the binding pocket, one may design new ligands that will interact favorably with the enzyme. With such structural information, available computational methods may be used to predict how strong the ligand binding interaction will be. Such methods thus enable the design of inhibitors that bind strongly, as well as selectively to the target enzyme.

Despite the fact that the genes for TAK1 has been isolated and the amino acid sequence of TAK1 is known, no one has described X-ray crystal structural coordinate information of TAK protein. As disclosed herein, such information would be extremely useful in identifying and designing potential inhibitors of the TAK kinase or homologues thereof, which, in turn, could have therapeutic utility.

The structures of several serine/threonine kinases have been solved by X-ray diffraction and analyzed. Specifically, the crystal structures of P38 kinase_(Wilson et al., J. Biol. Chem., 271, pp. 27696-27700 (1996)) and MAPKAP Kinase 2 (U.S. Provisional application 60/337,513) have been studied in detail,

To date, no crystal structures of TAK kinase have been reported. Thus the crystal structure of unphosphorylated TAK kinase domain complexes with inhibitors are of great importance for defining the active conformation of TAK kinase. This information is essential for the rational design of selective and potent inhibitors of TAK.

### SUMMARY OF THE INVENTION

The present invention provides for the first time, crystallizable compositions, crystals, and the crystal structures of a TAK1—inhibitor complex. The TAK1 protein used in these studies corresponds to a single polypeptide chain, which encompasses the complete catalytic kinase domain, amino acids 31 to 303 fused to the C-terminal 36 amino acids of TAB1 (468 to 504). Solving this crystal structure has allowed the applicants to determine the key structural features of TAK1, particularly the shape of its substrate and ATP-binding pockets.

Thus, in one aspect, the present invention provides molecules or molecular complexes comprising all or parts of these binding pockets, or homologues of these binding pockets that have similar three-dimensional shapes.

In another aspect, the present invention further provides crystals of TAK1 complexed with adenosine and methods for producing these crystals. In this embodiment, TAK1 is unphosphorylated.

In a further aspect, the present invention provides crystallizable compositions from which TAK1-ligand complexes may be obtained.

In another aspect, the present invention provides for a general strategy for the design of protein constructs for producing crystallisable kinase domains.

In another aspect, the invention provides a data storage medium that comprises the structure coordinates of molecules and molecular complexes that comprise all or part of the TAK1 binding pockets. Such storage medium encoded with these data when read and utilized by a computer programmed with appropriate software displays, on a computer screen or similar viewing device, a three-dimensional graphical representation of a molecule or molecular complex comprising such binding pockets or similarly shaped homologous binding pockets.

In yet another aspect, the invention provides computational methods of using structure coordinates of the TAK1 complex to screen for and design compounds, including inhibitory compounds and antibodies that interact with TAK1 or homologues thereof. In certain embodiments, the invention provides methods for designing, evaluating and identifying compounds, which bind to the aforementioned binding pockets. In certain embodiments, such compounds are potential inhibitors of TAK1 or their homologues.

In a further aspect, the invention provides a method for determining at least a portion of the three-dimensional structure of molecules or molecular complexes which contain at least some structurally similar features to TAK1, particularly ITK, LCK and MAPKAP kinase2 and their homologues. In certain embodiments, this is achieved by using at least some of the structural coordinates obtained from the TAK1 complexes.

### BRIEF DESCRIPTION OF THE FIGURES

FIG. 1 lists the atomic structure coordinates for the unphosphorylated TAK1-9-β-D-Ribofuranosyladenine (“adenosine”) inhibitor complex as derived by X-ray diffraction from the crystal (Ser 27-Thr 178 of FIG. 1: SEQ ID NO: 14; Gly 191-Gln 303 of FIG. 1: SEQ ID NO: 15; His 468-Glu 497 of FIG. 1: SEQ ID NO: 16).

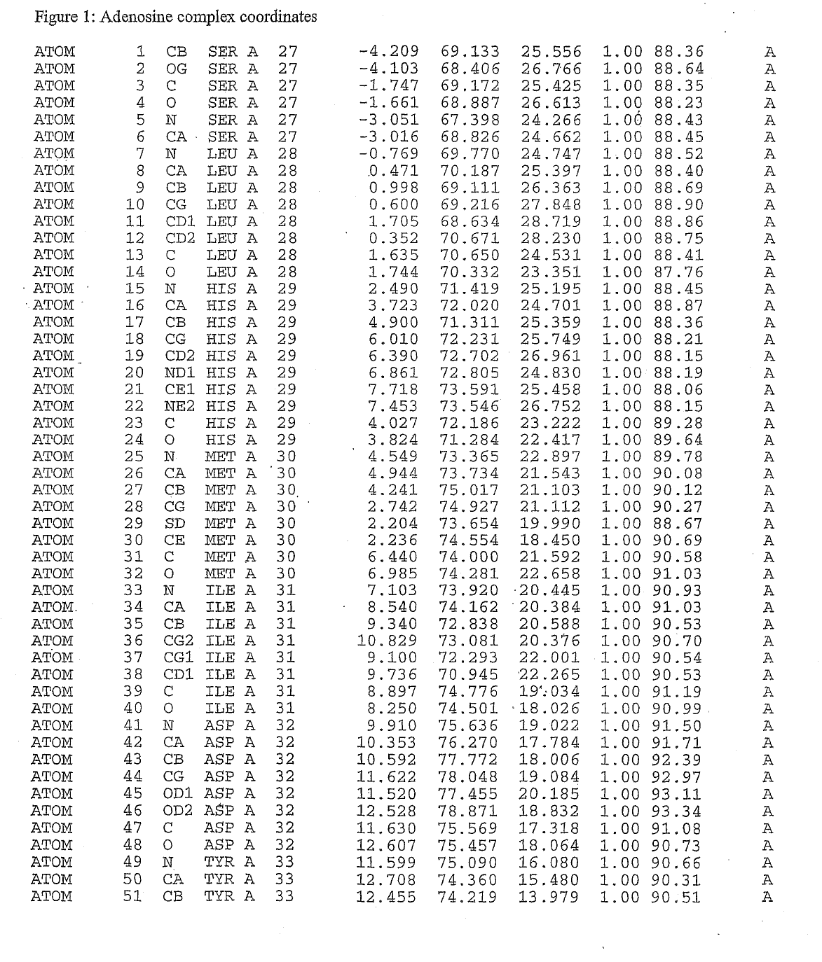

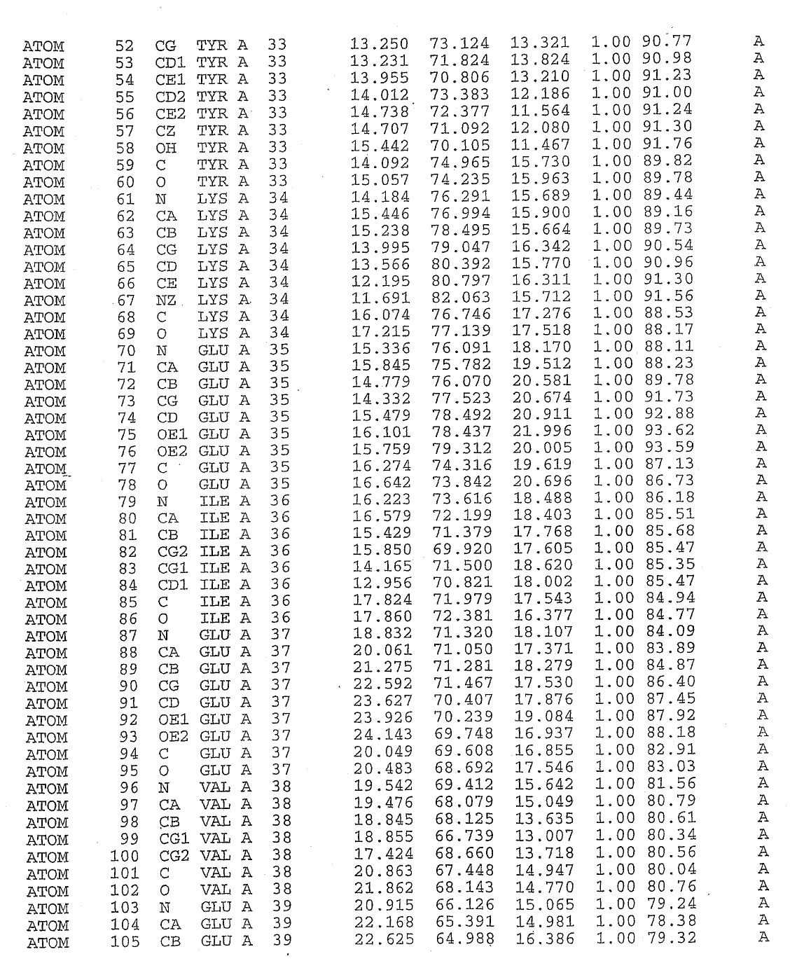

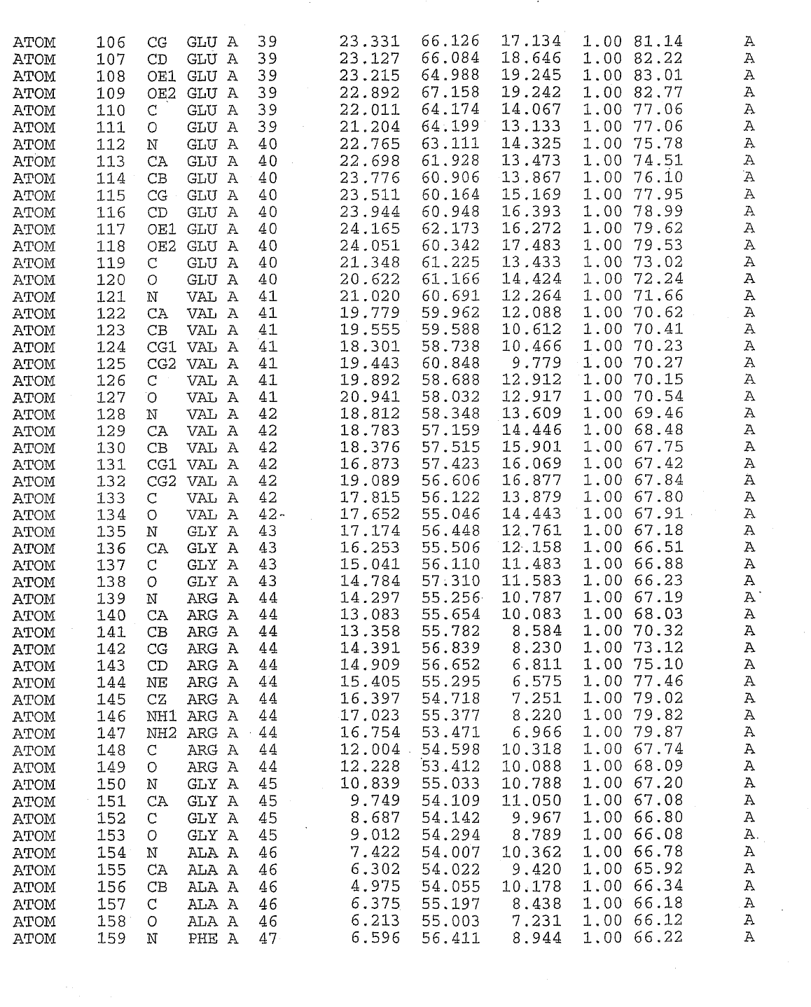

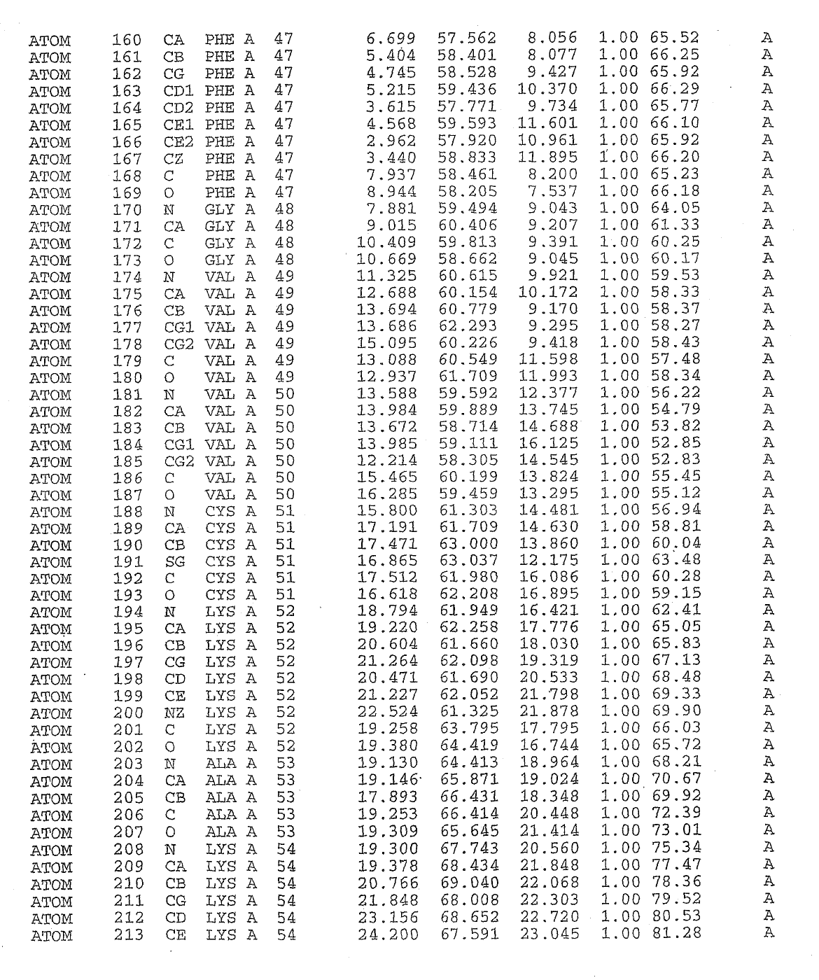

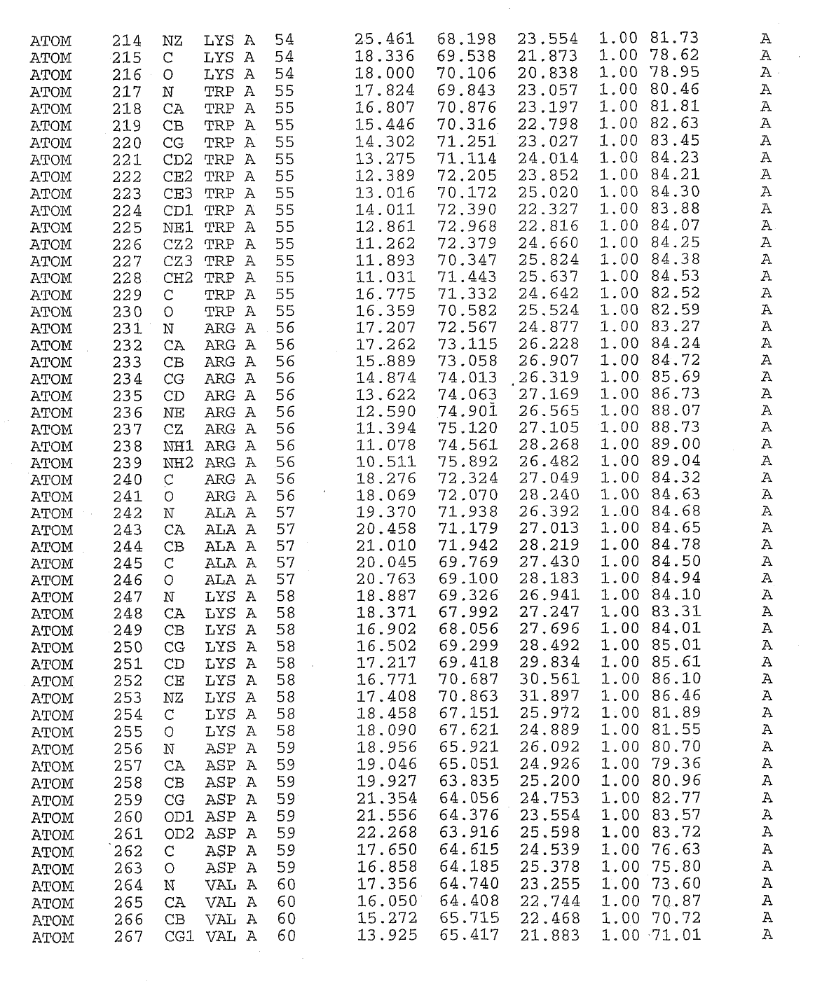

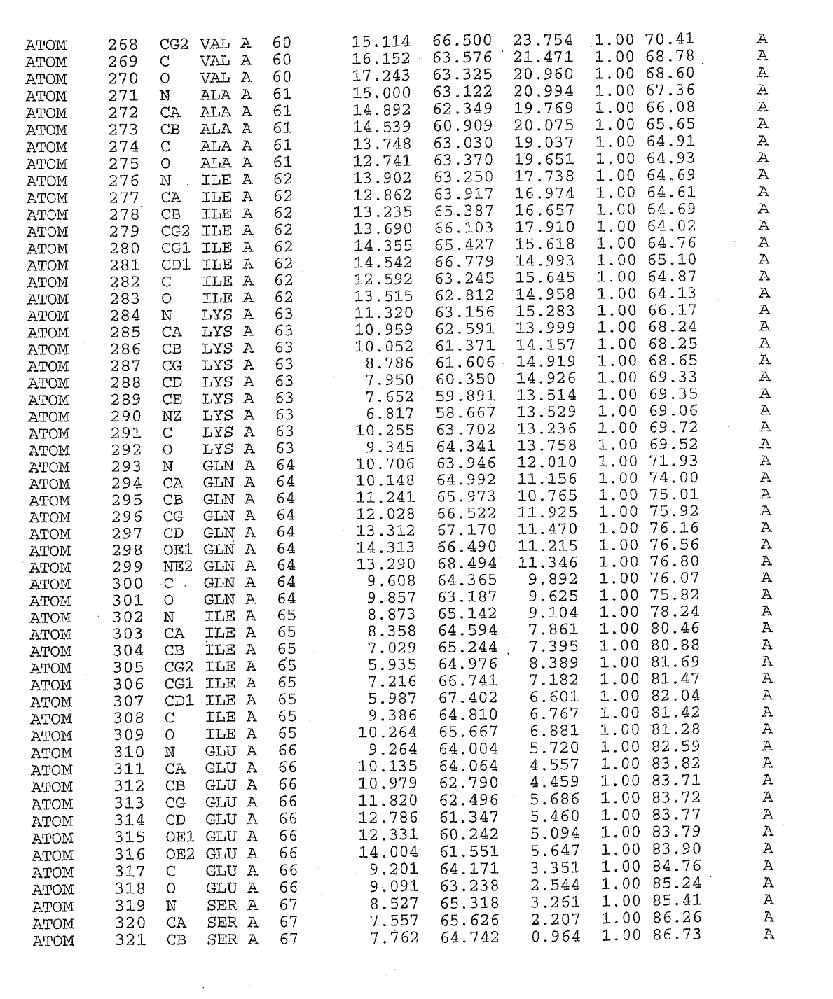

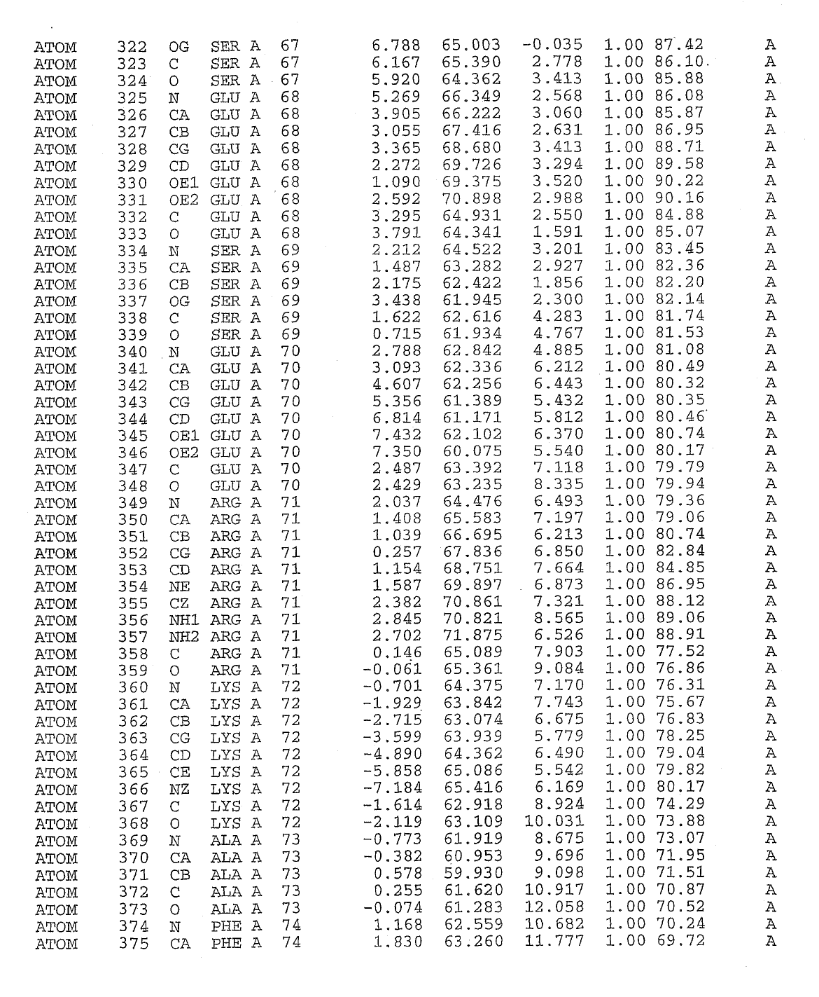

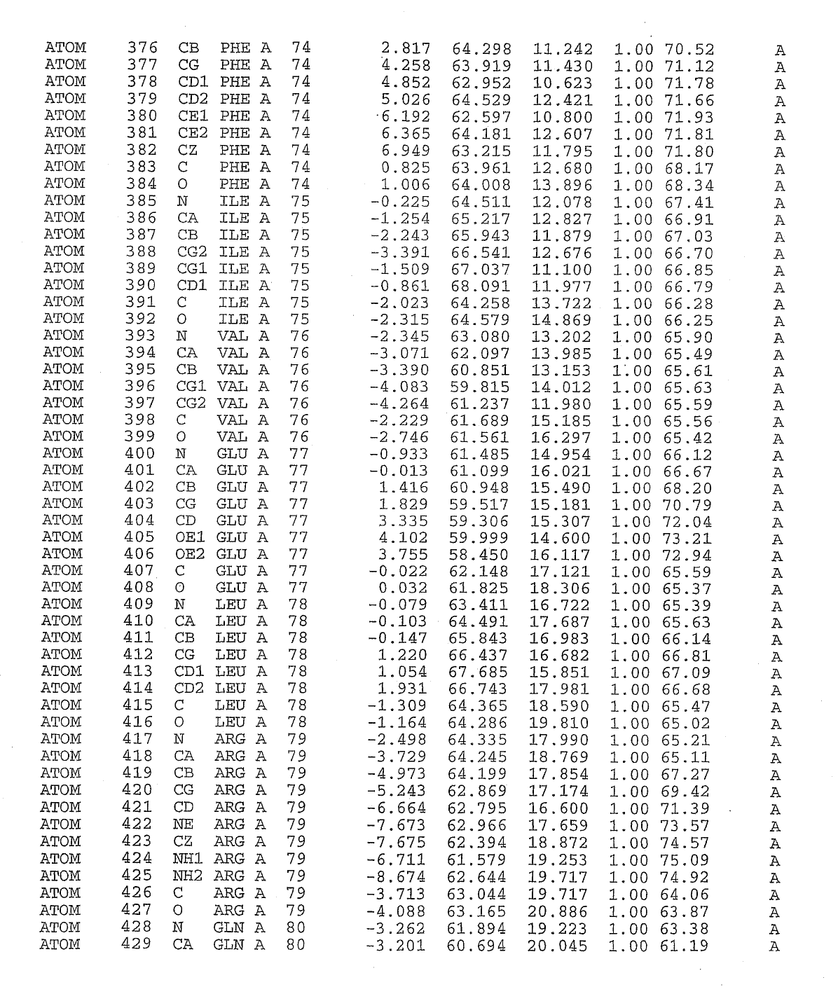

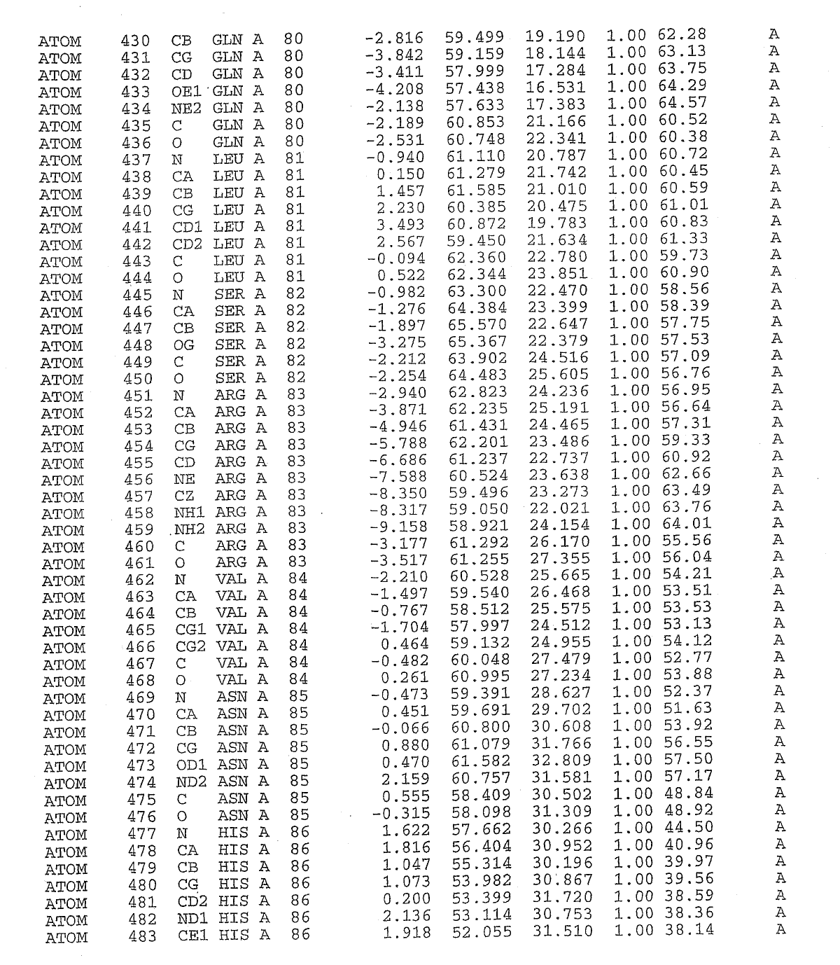

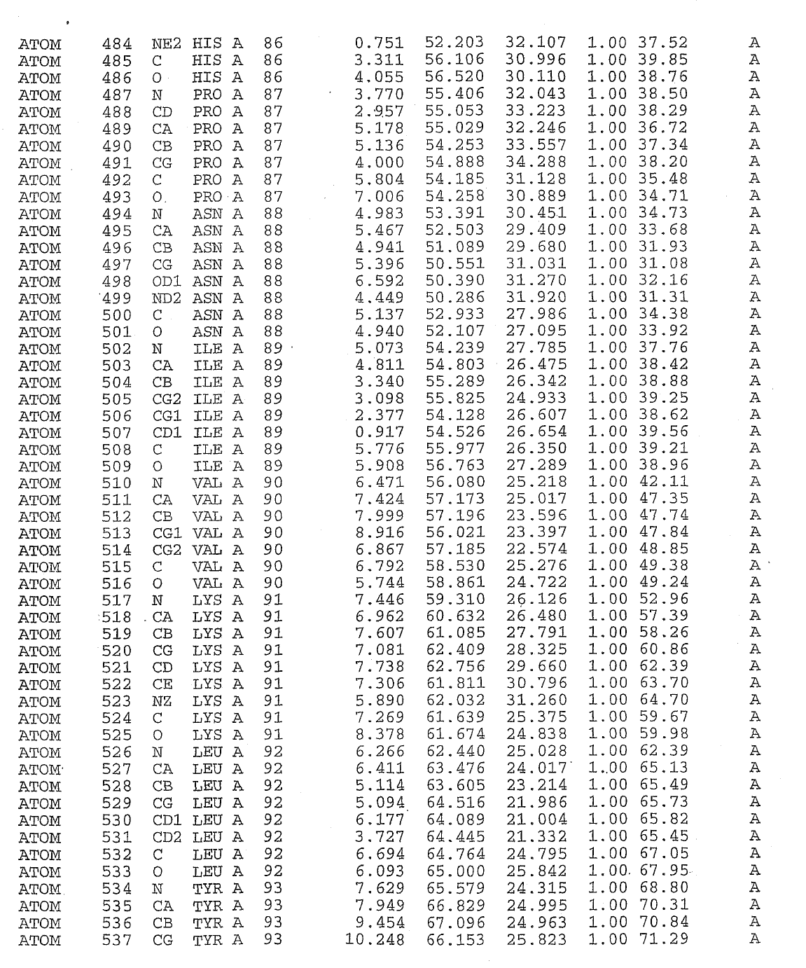

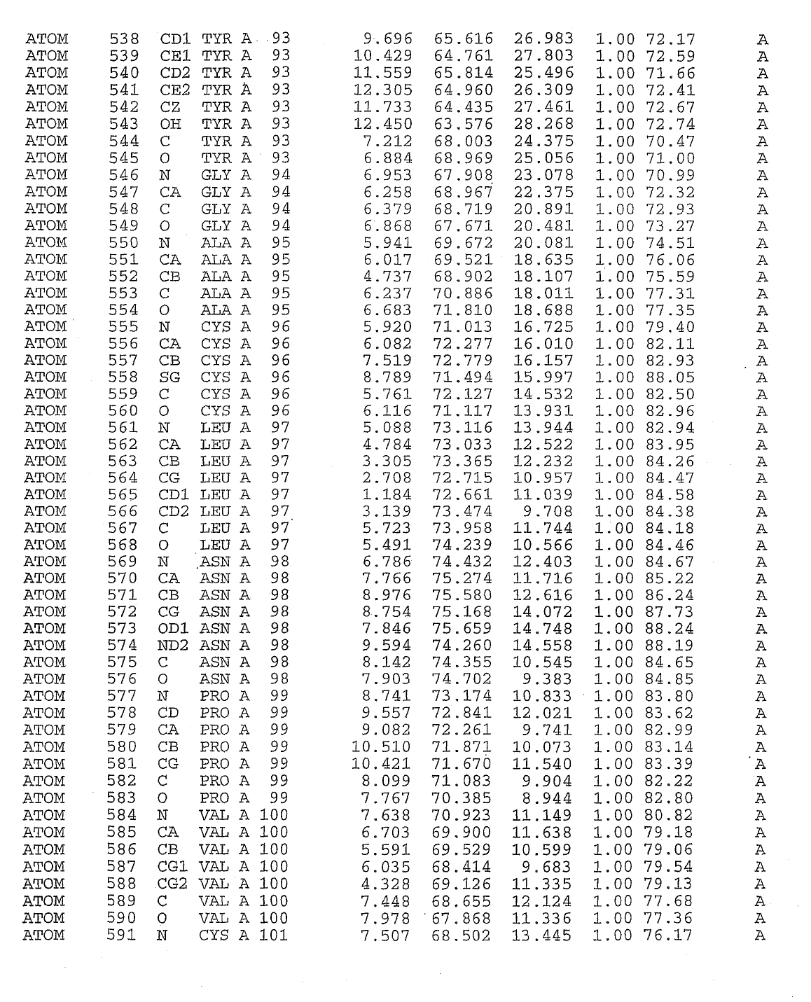

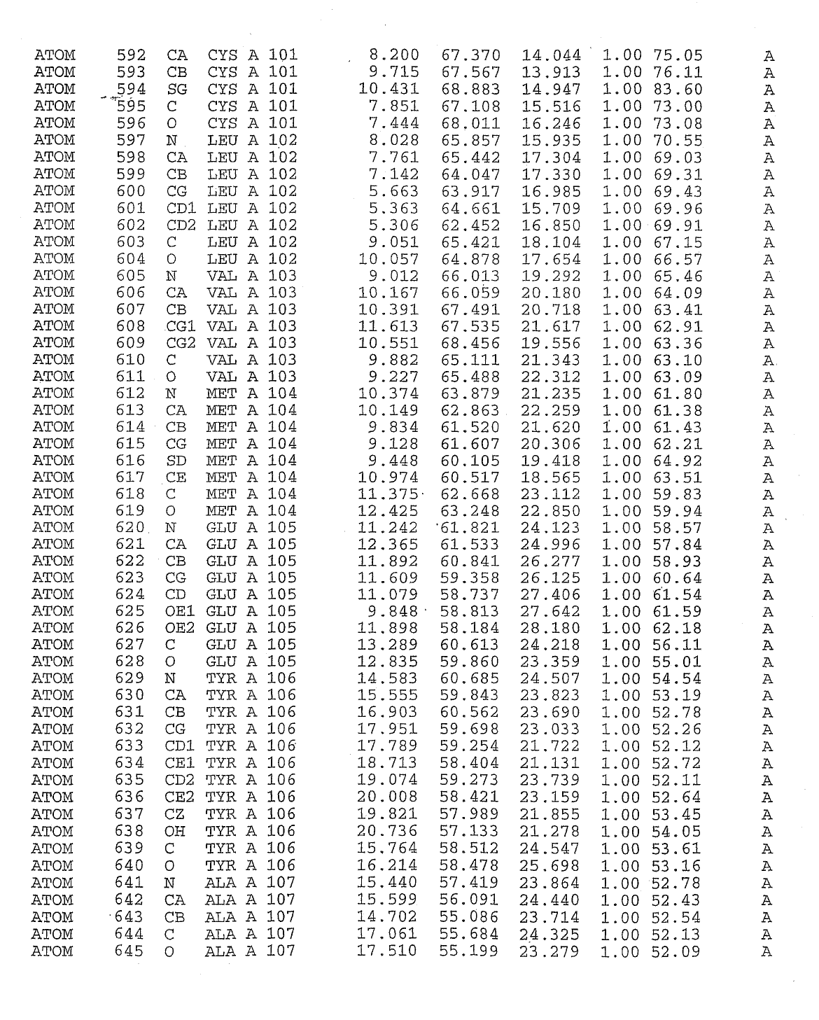

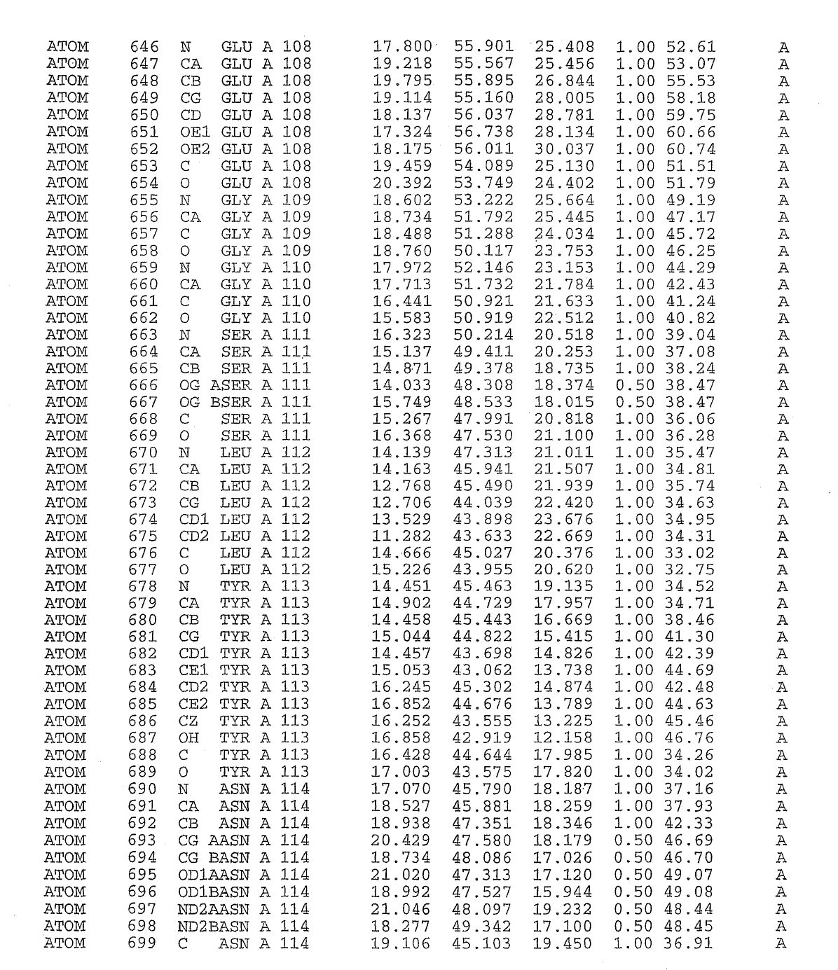

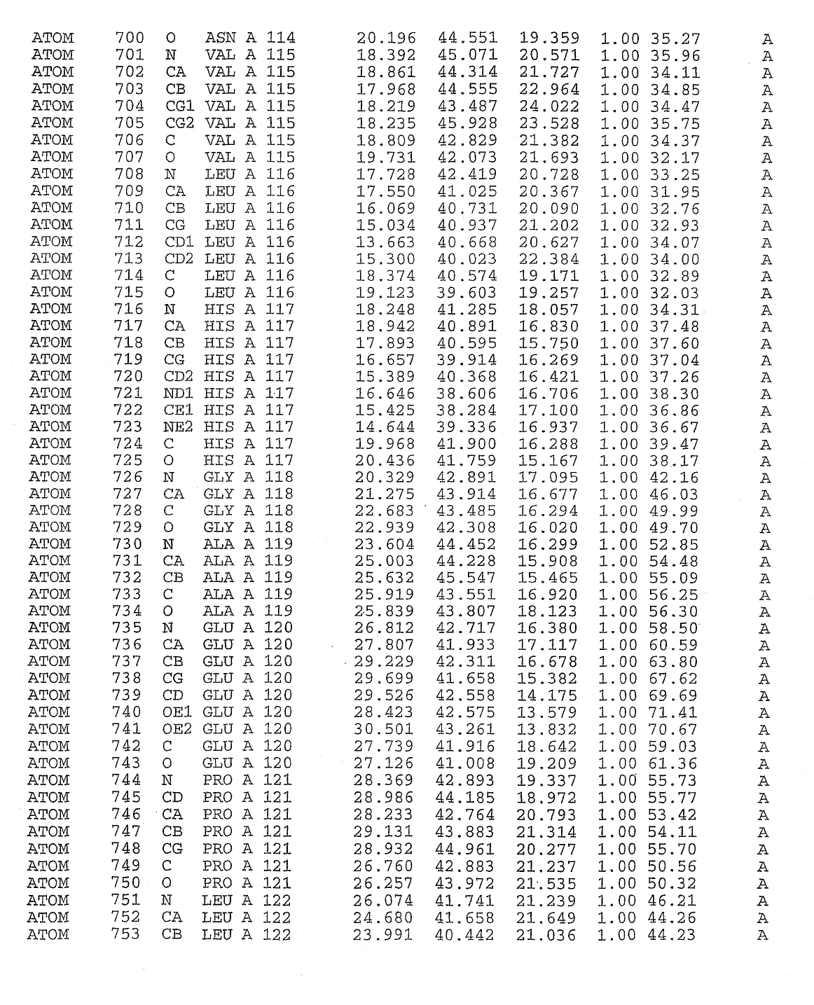

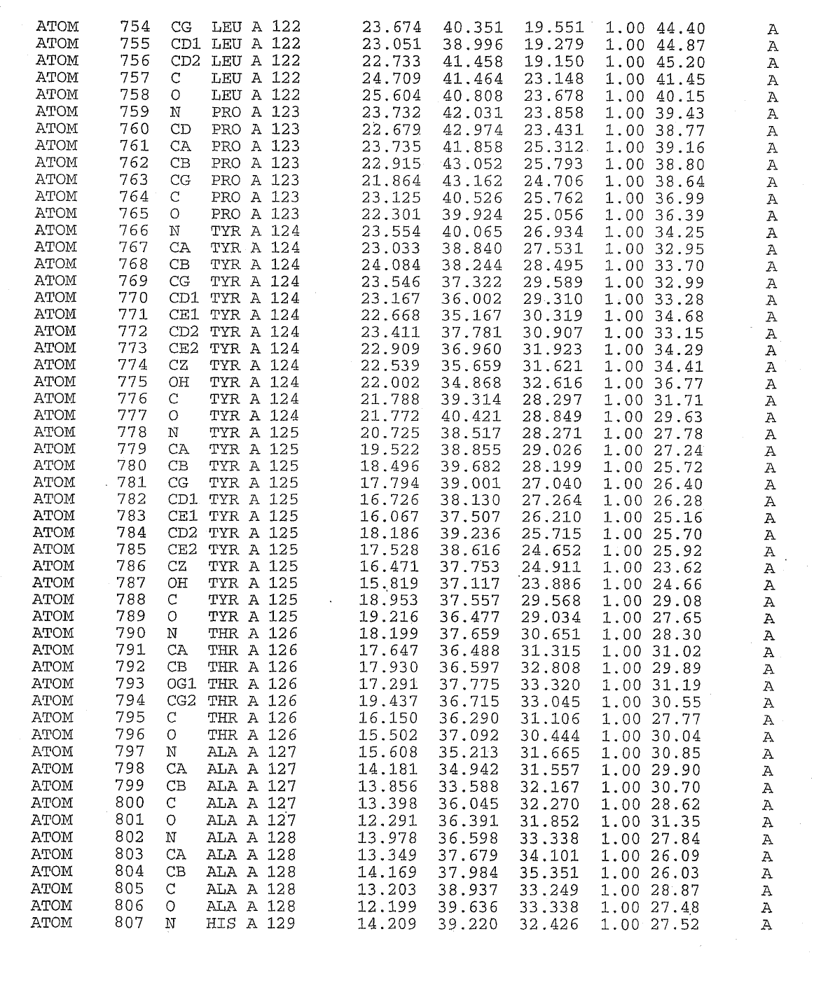

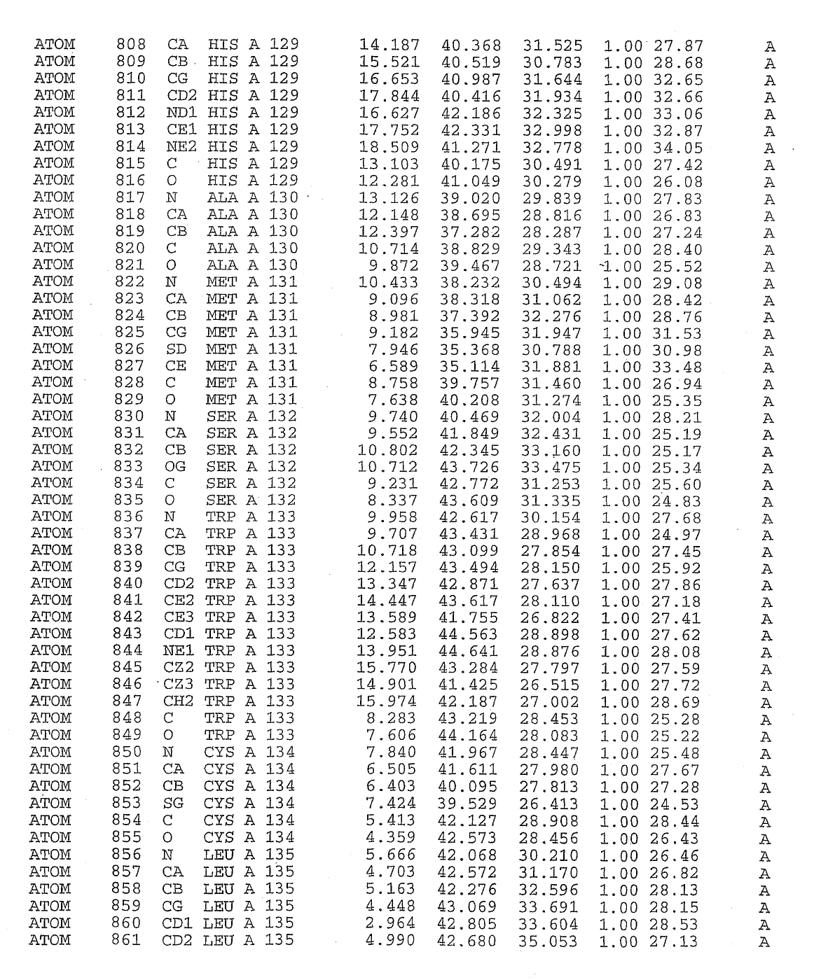

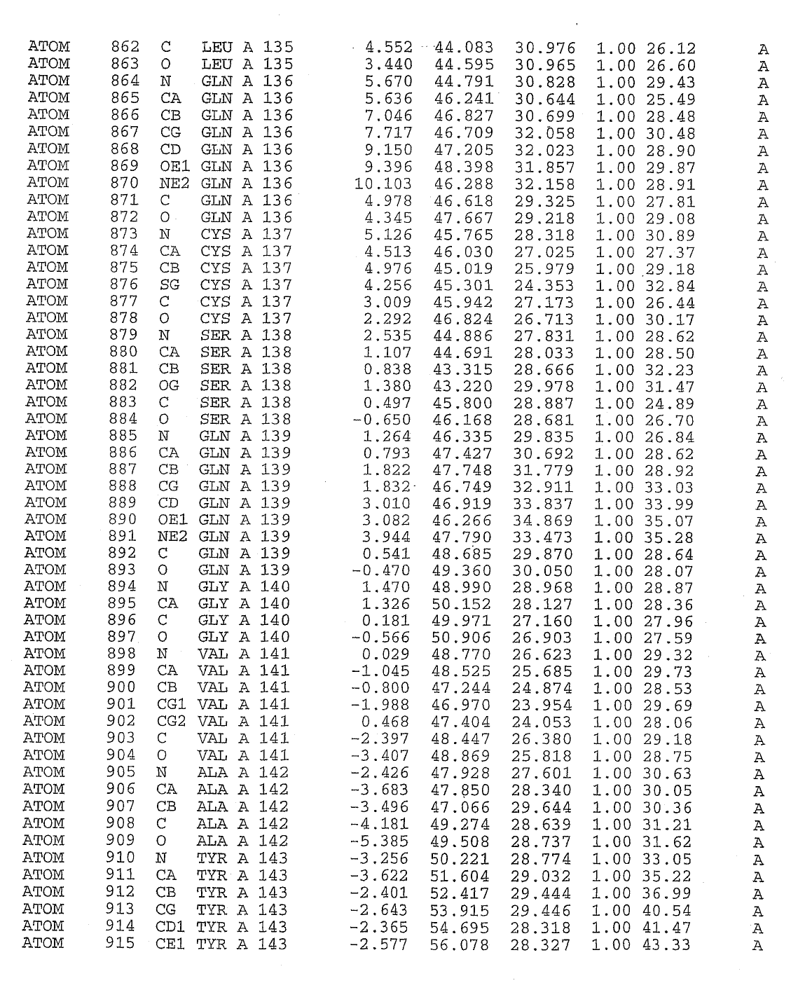

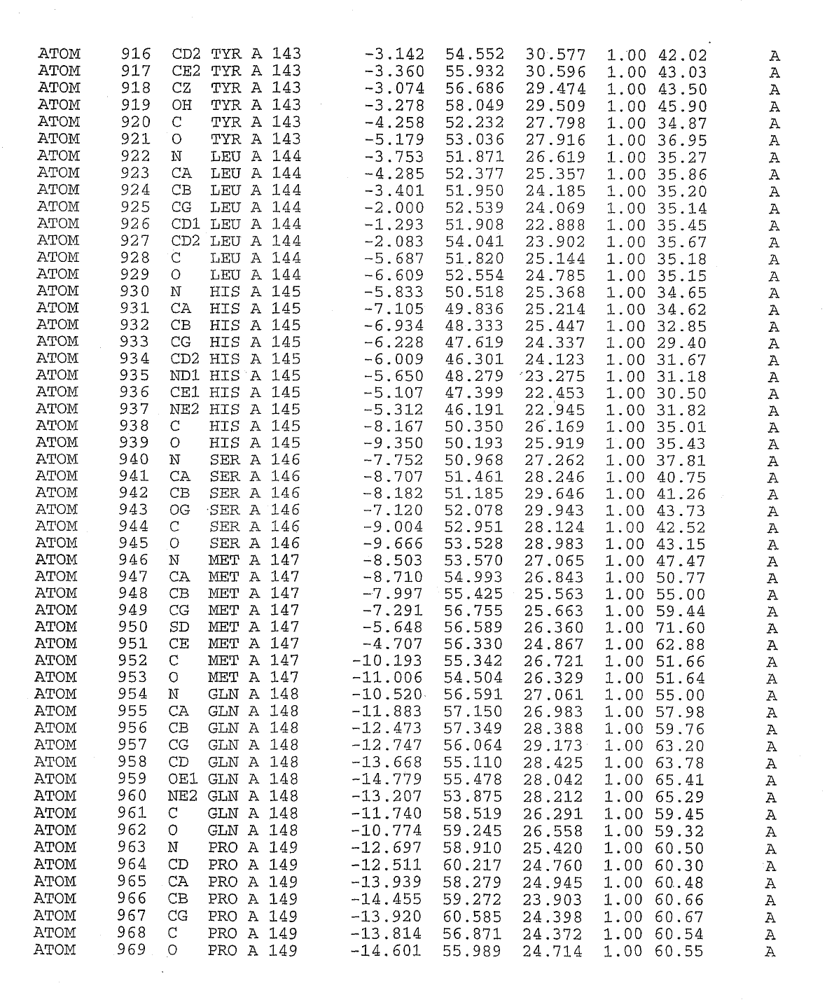

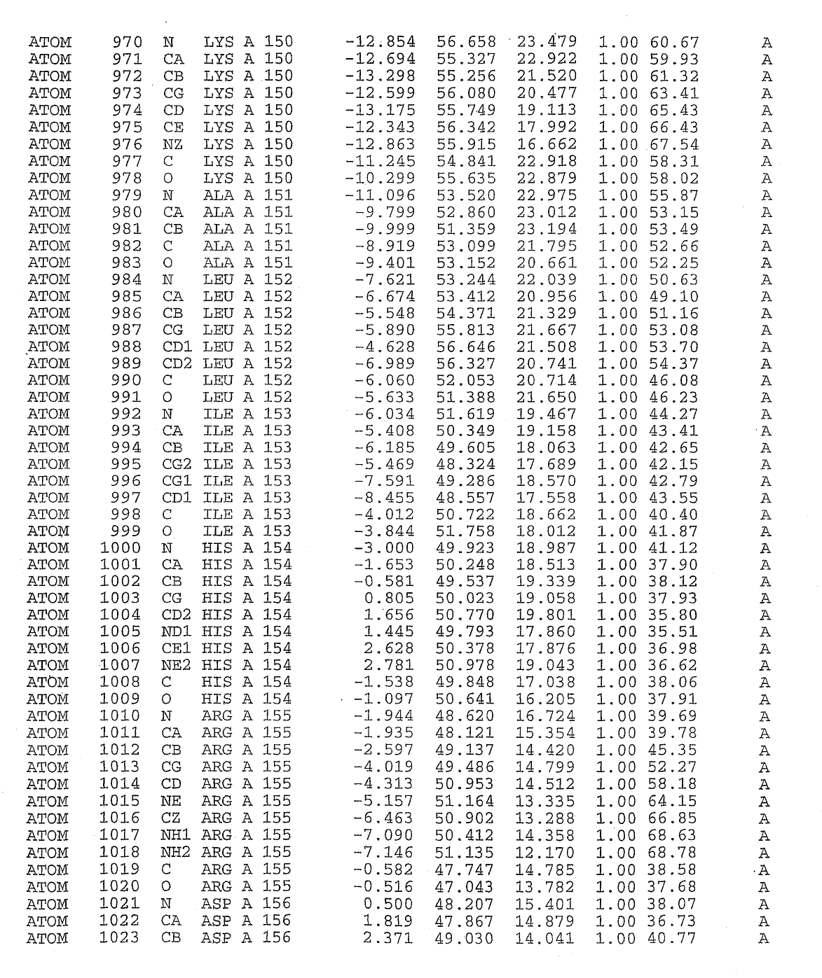

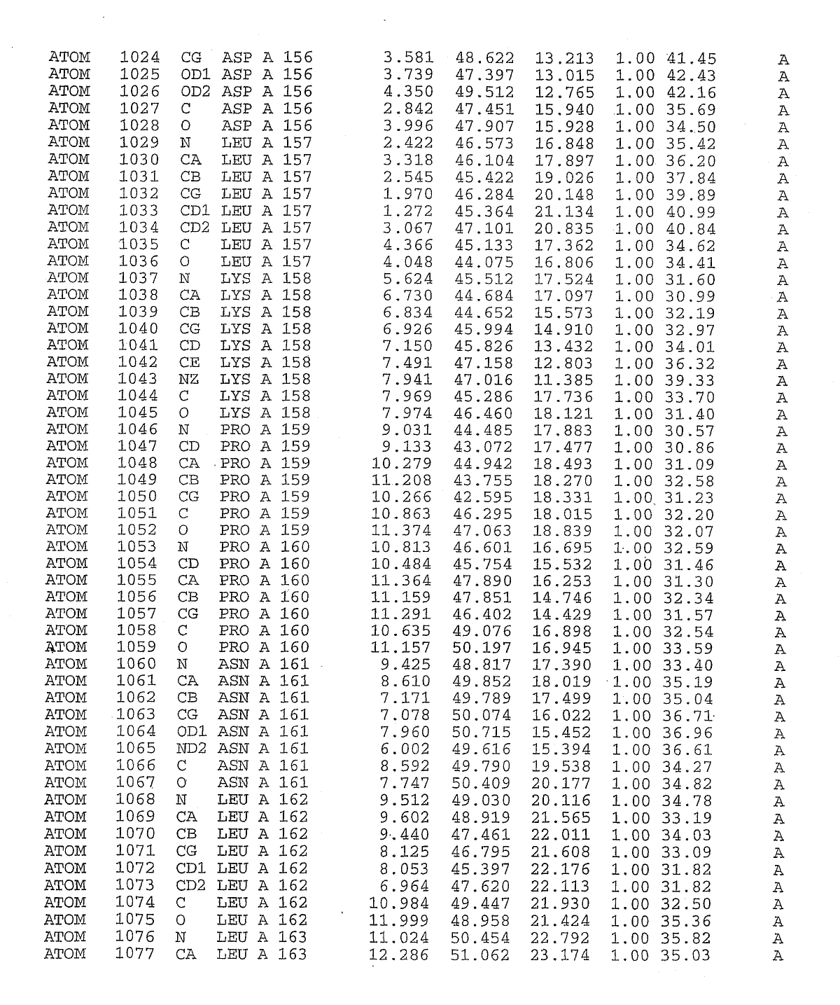

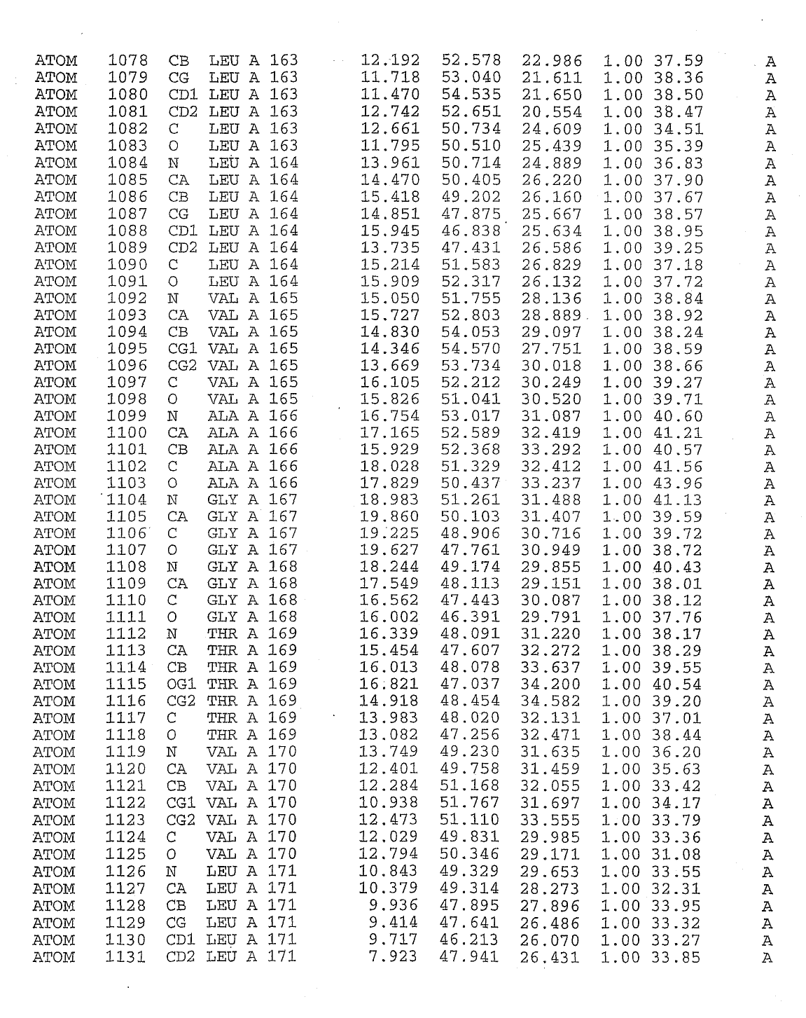

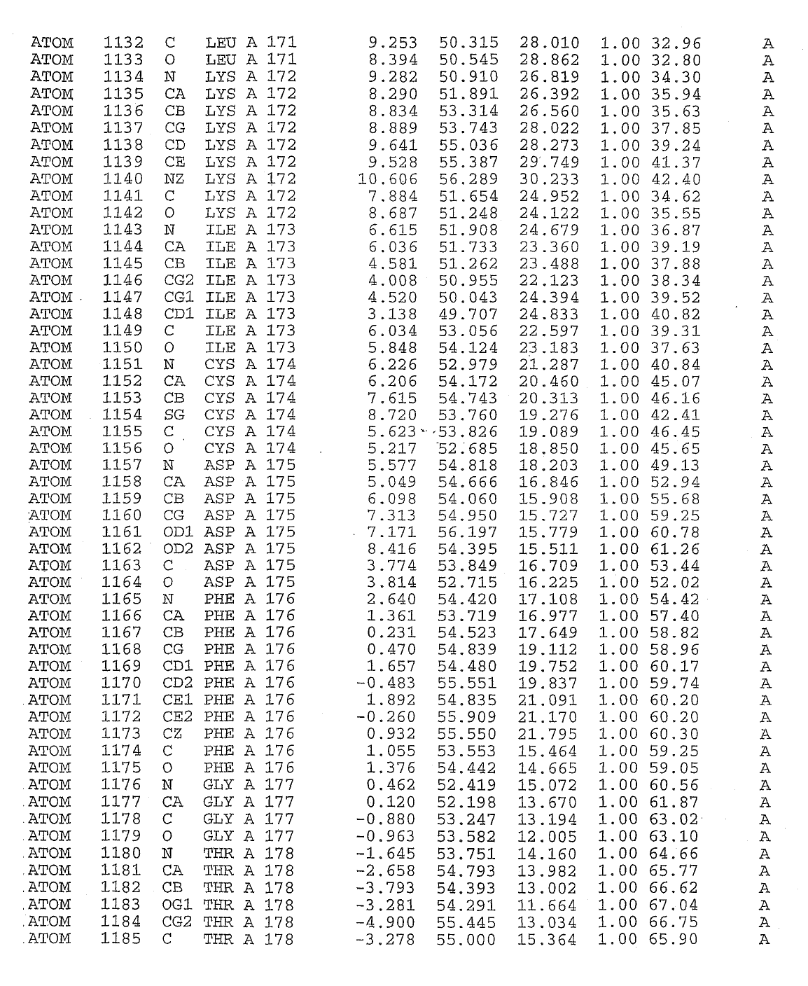

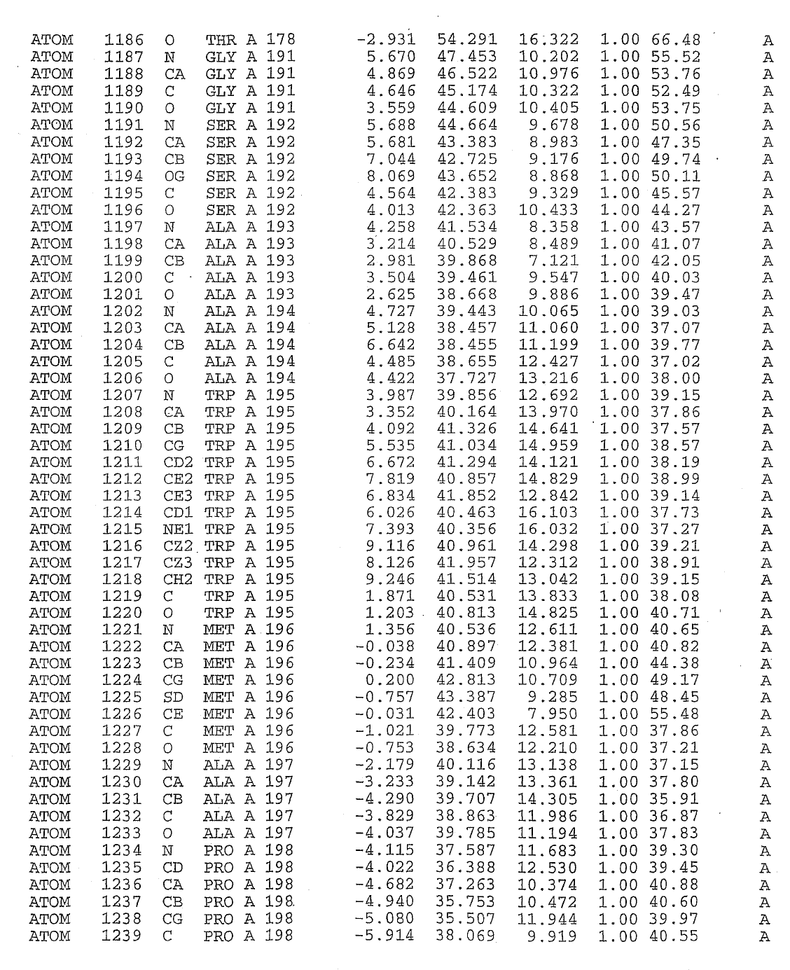

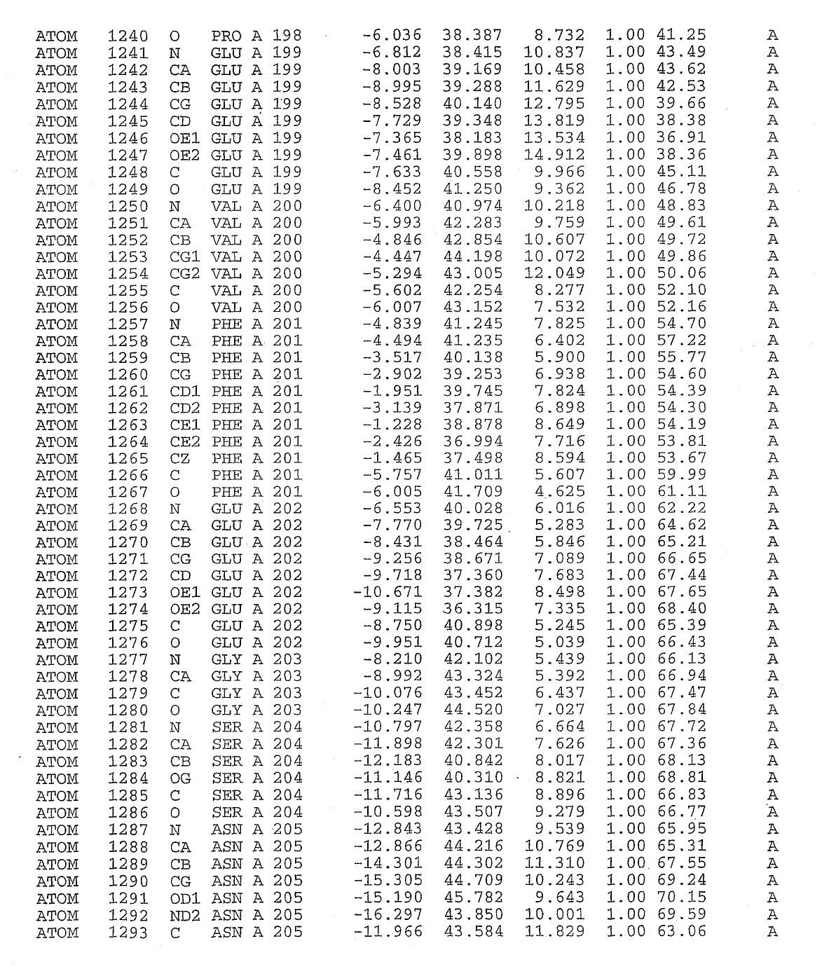

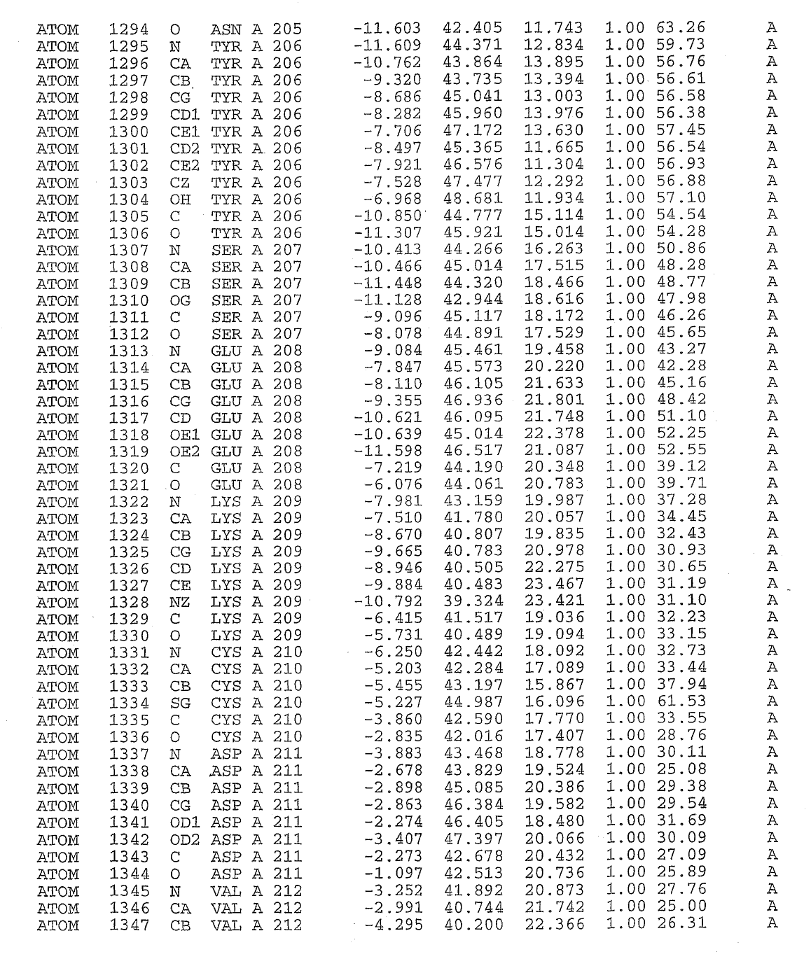

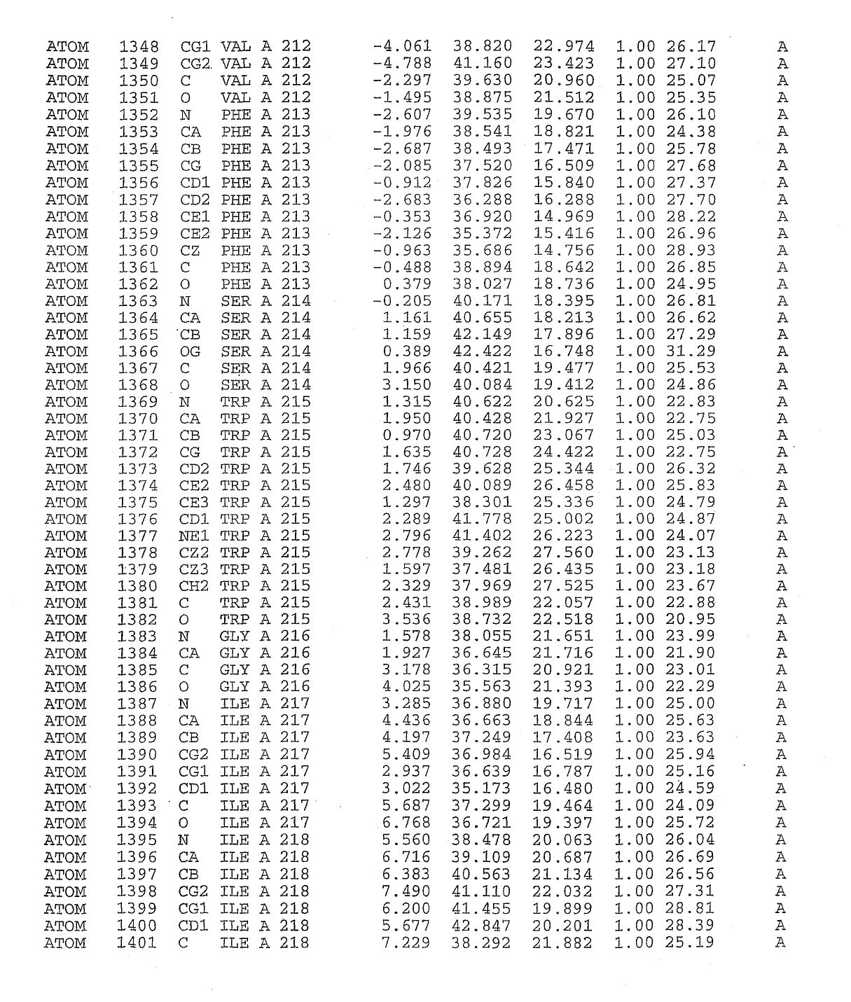

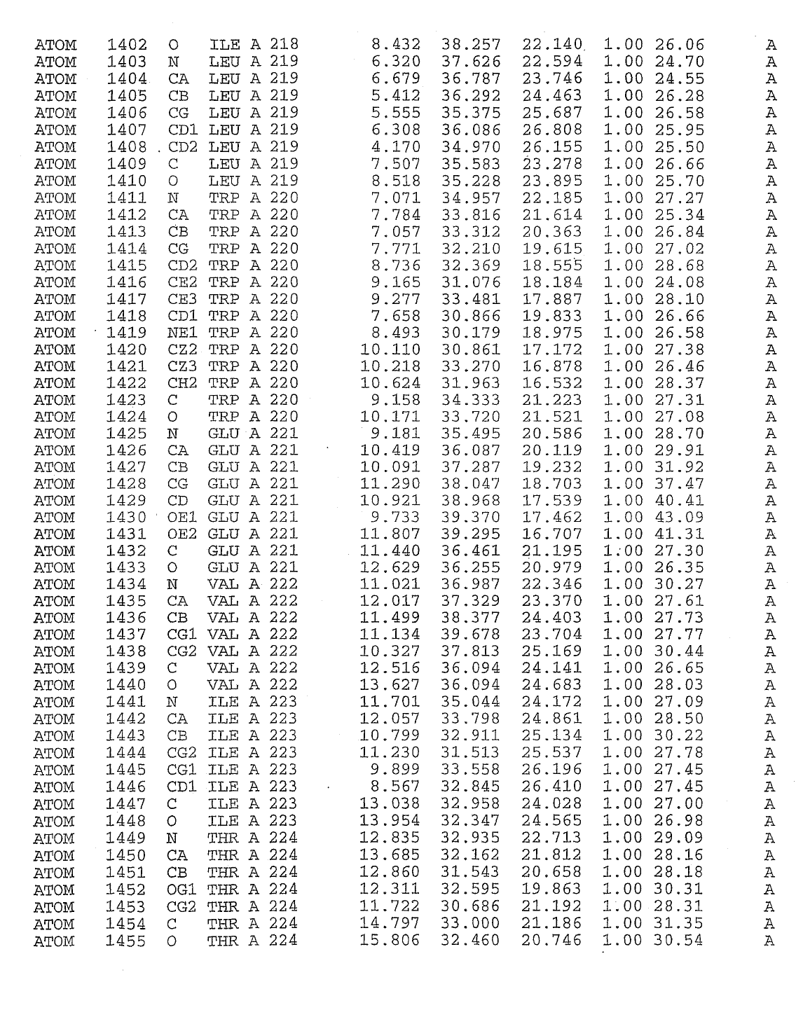

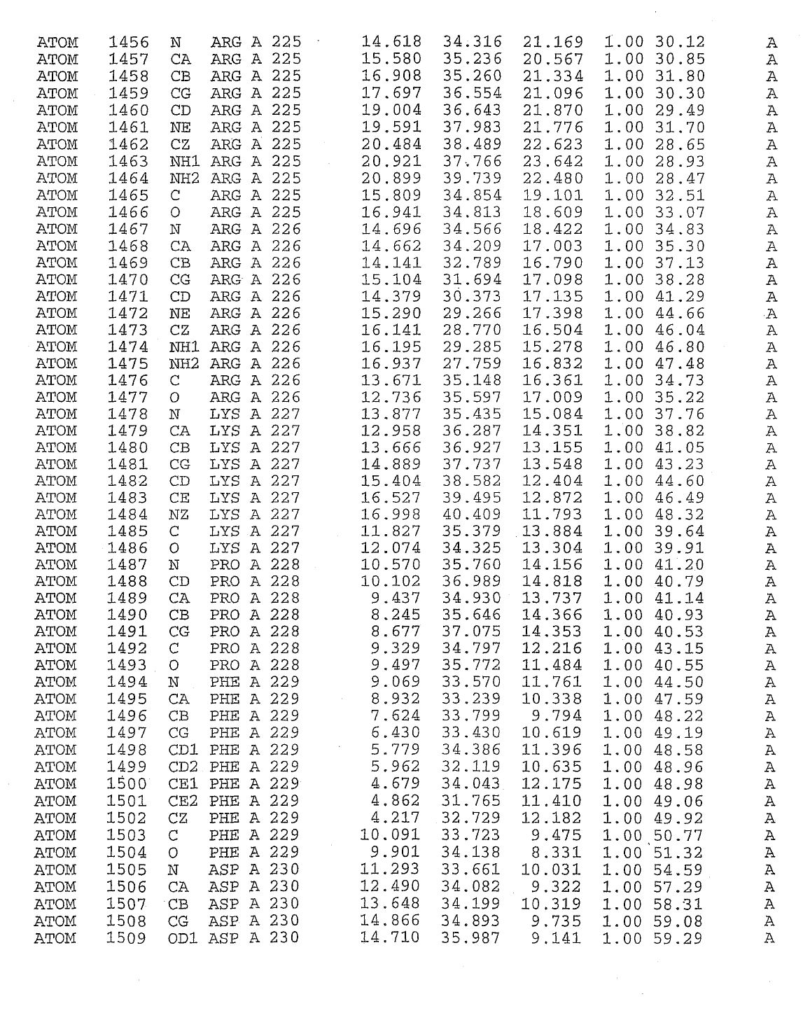

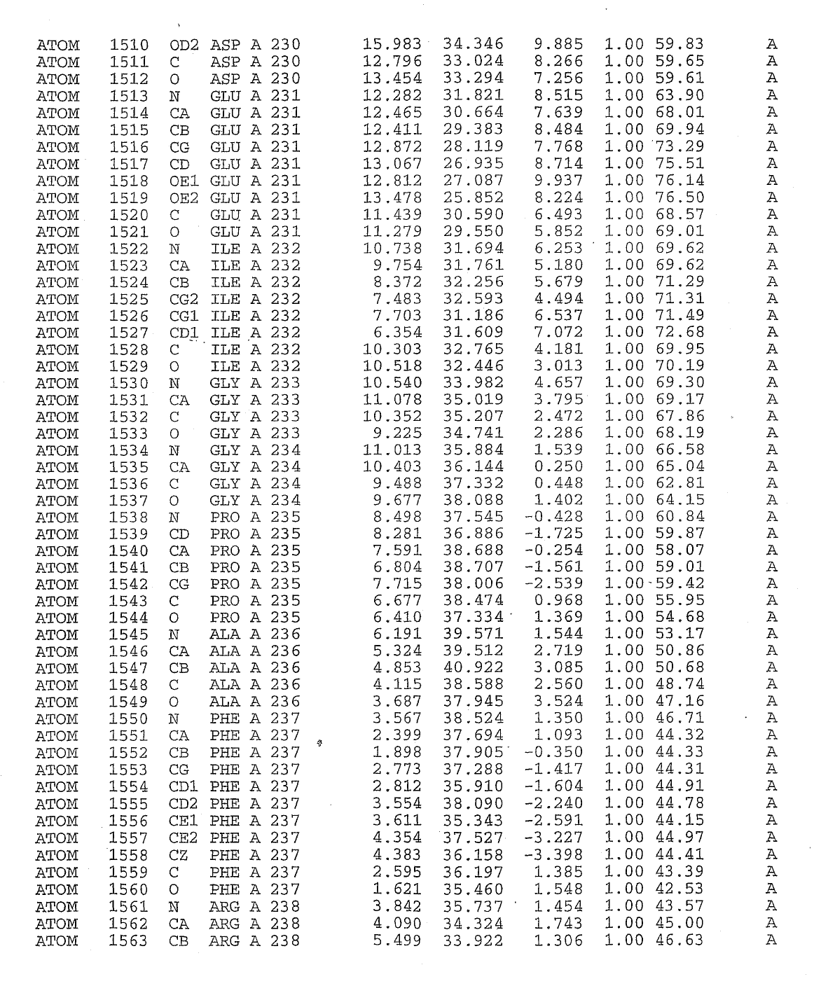

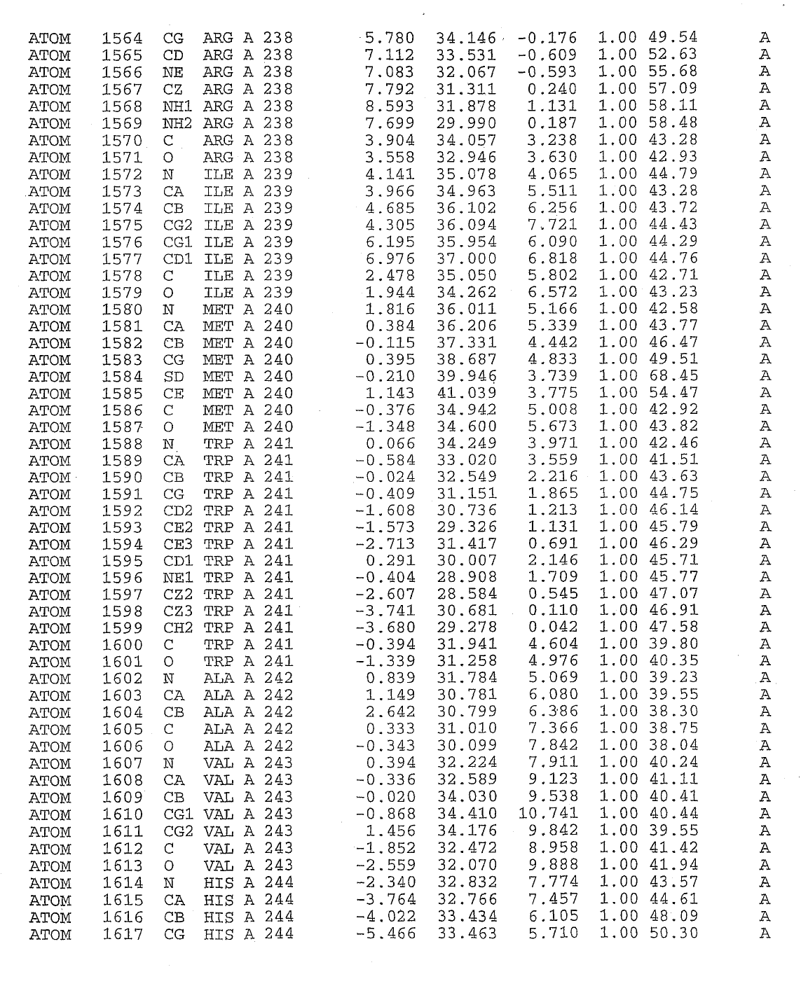

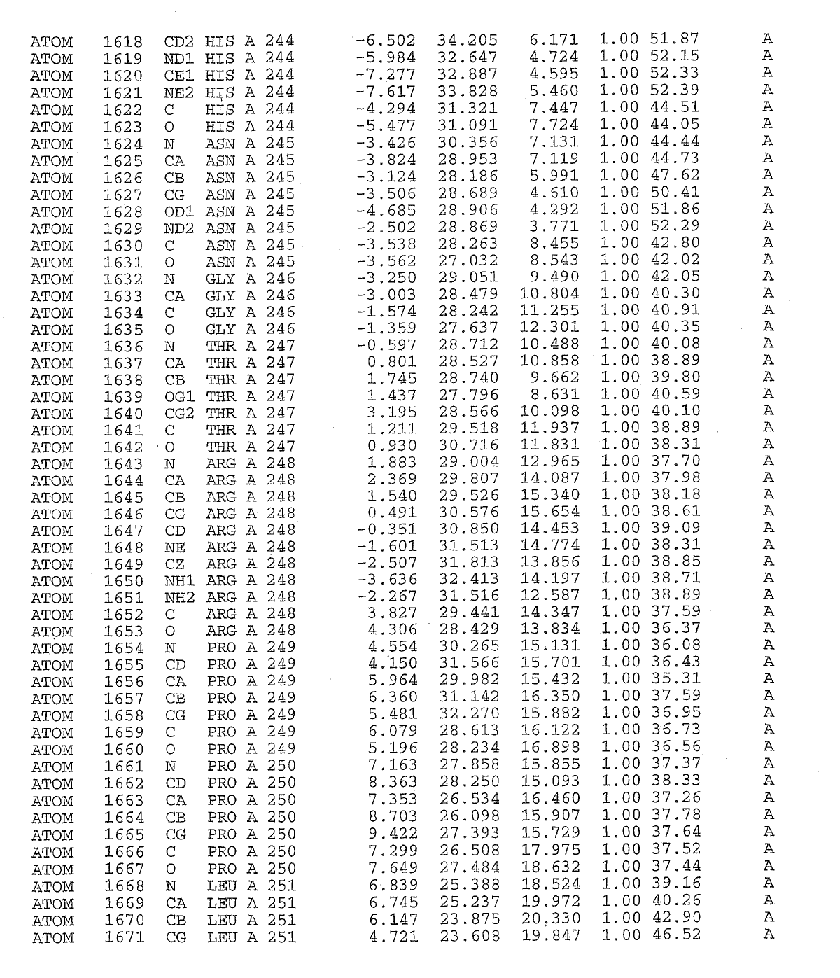

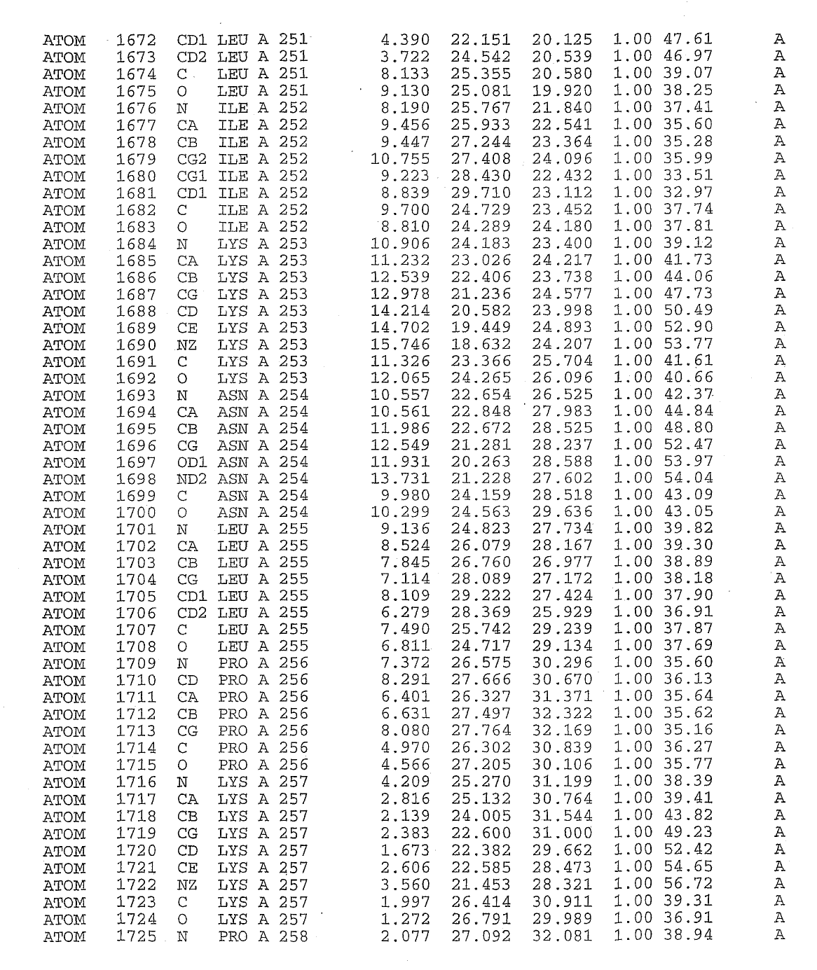

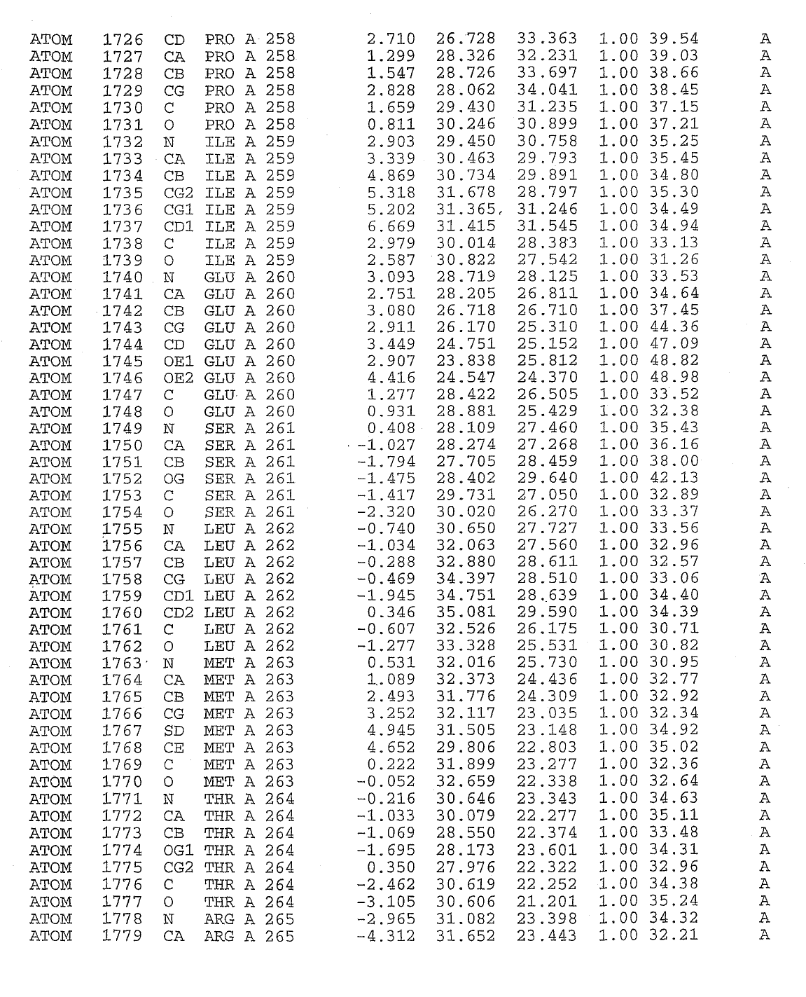

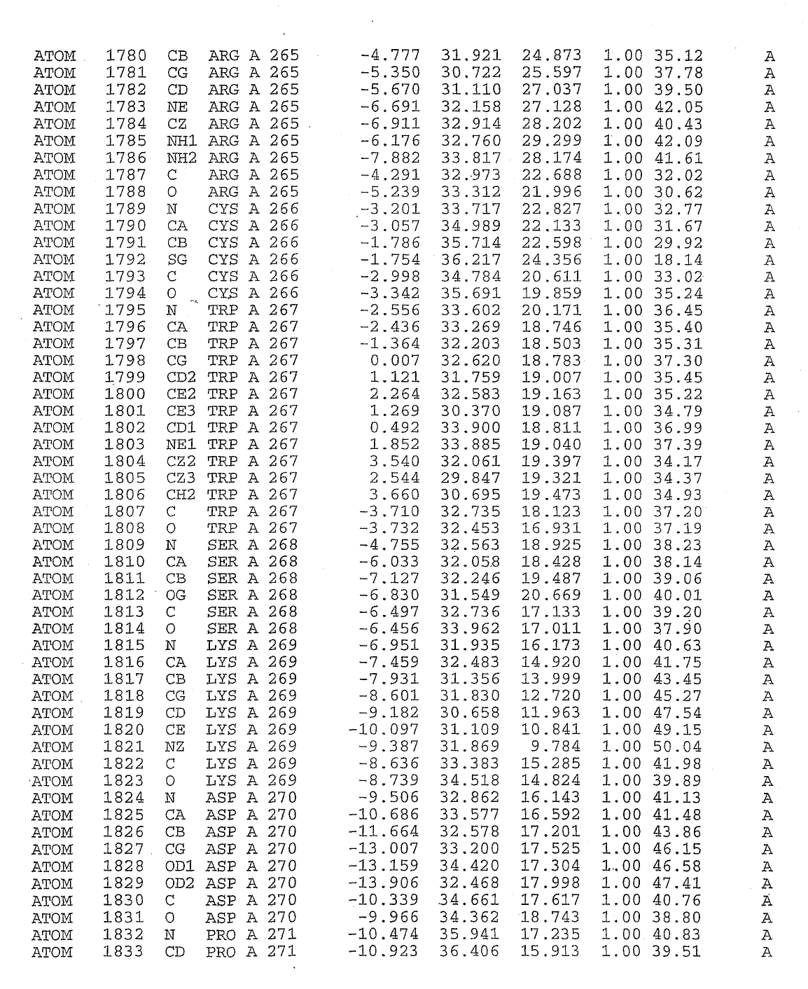

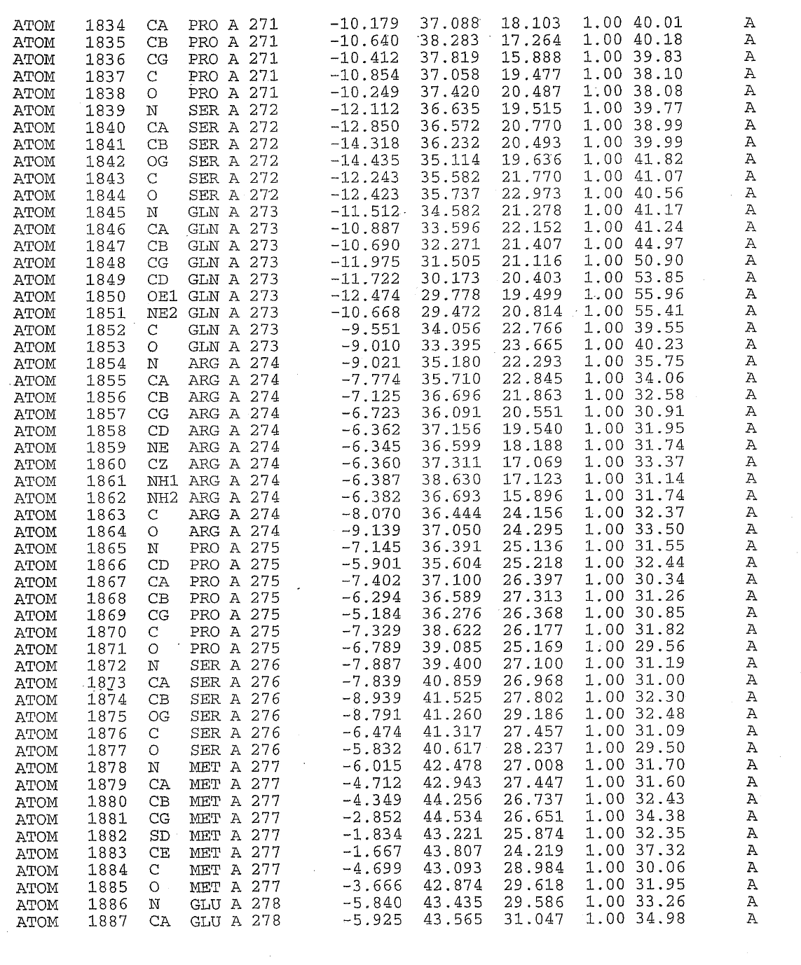

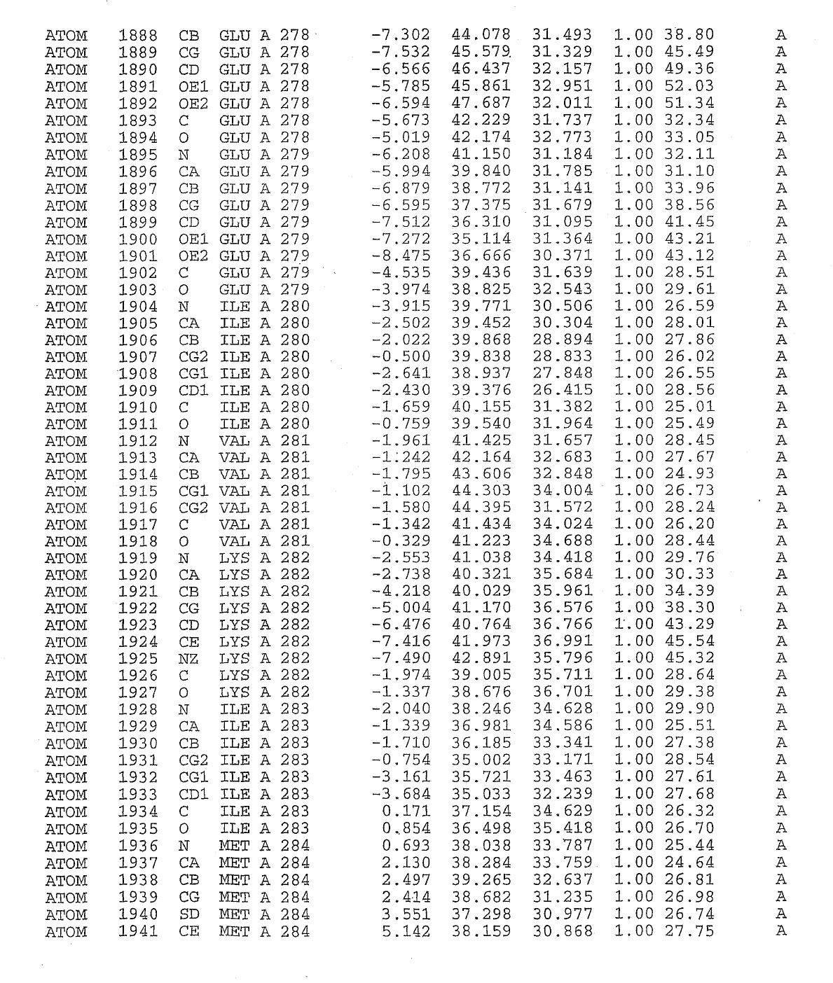

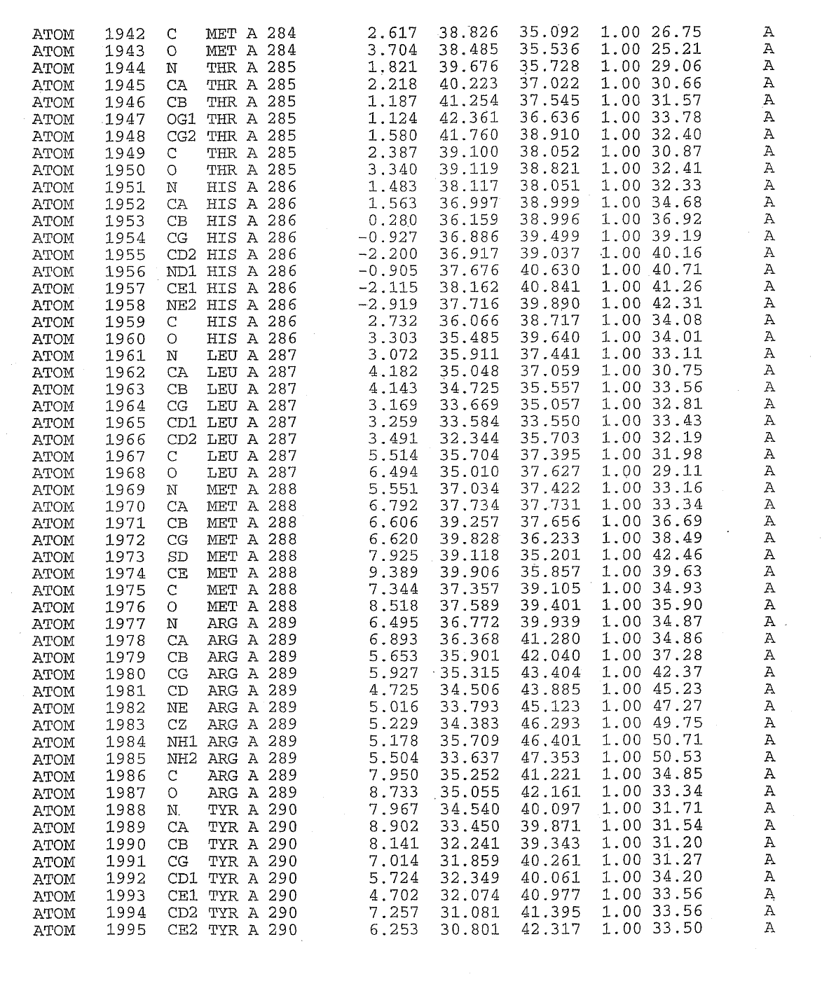

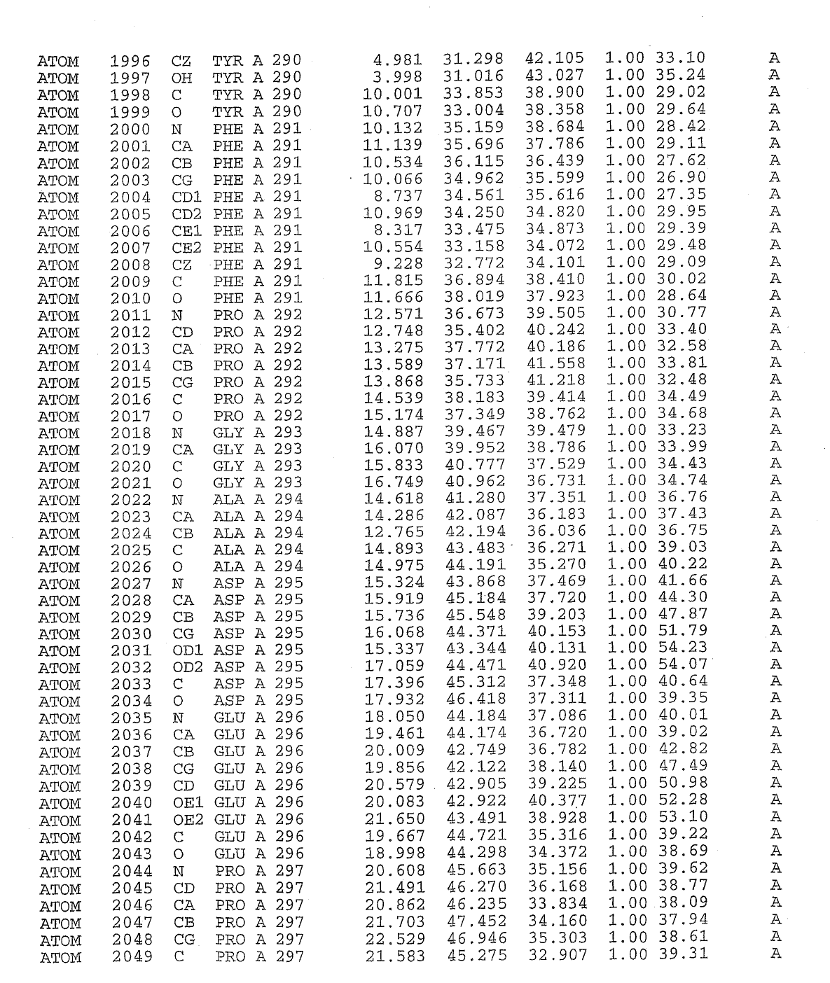

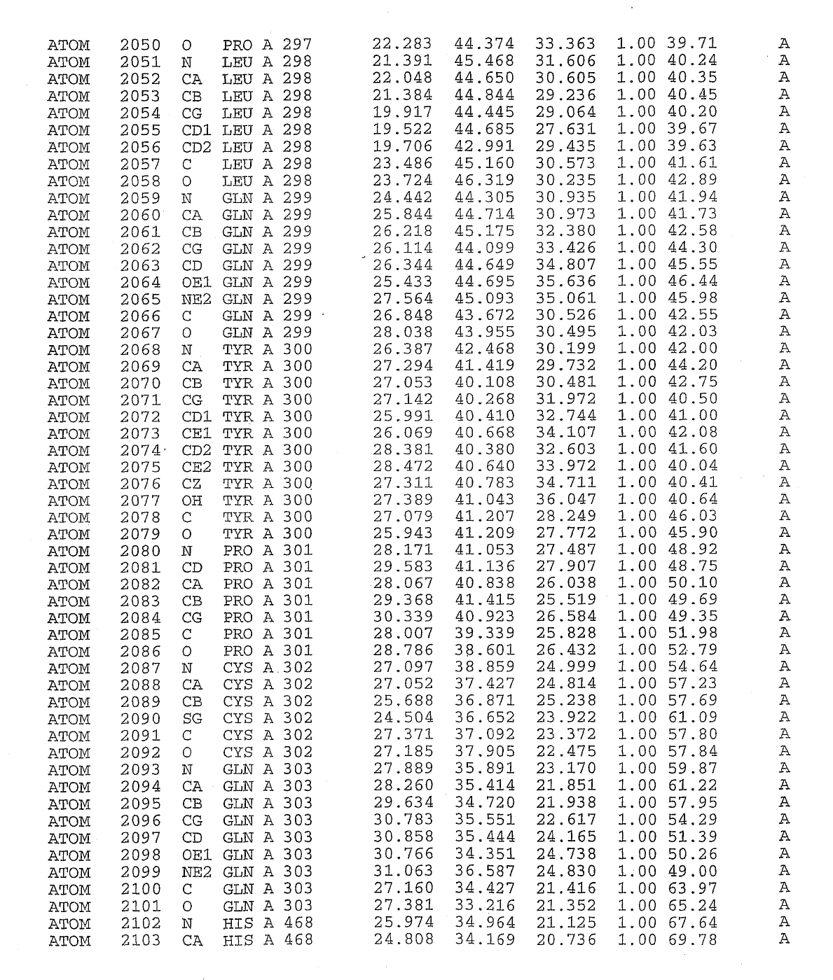

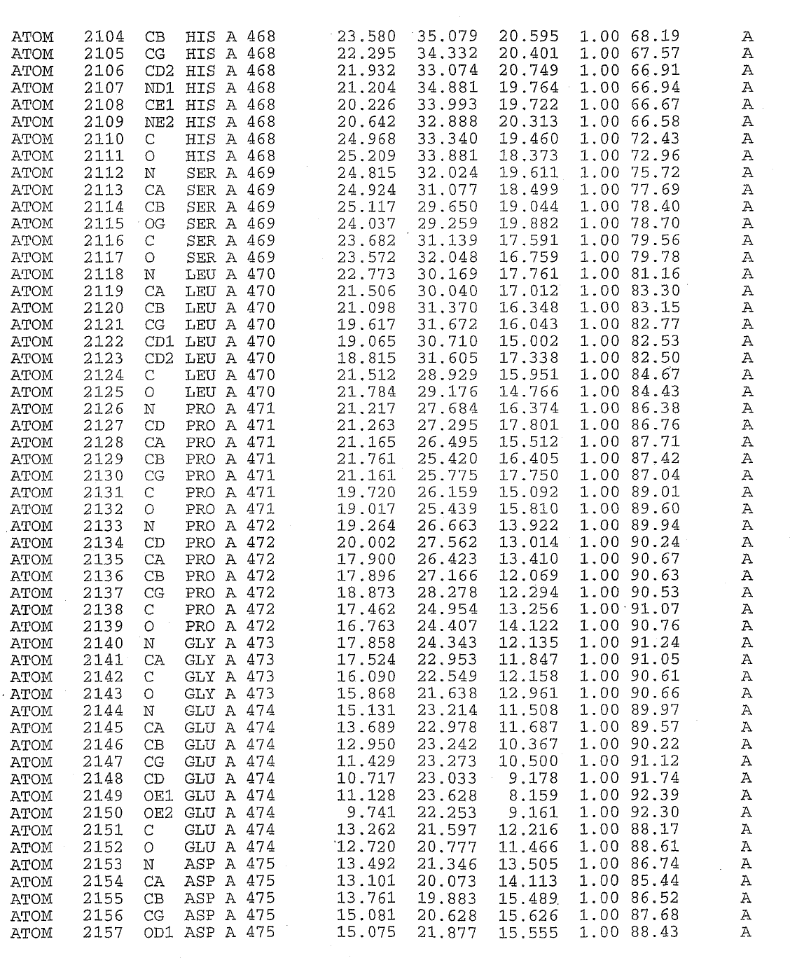

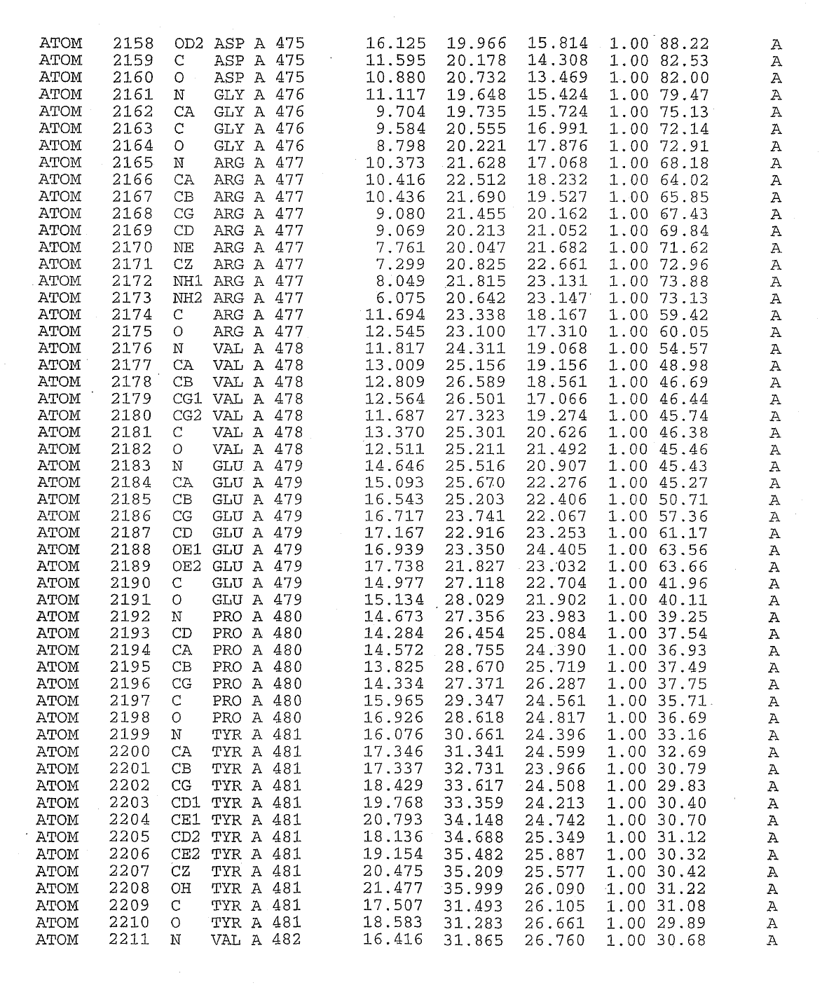

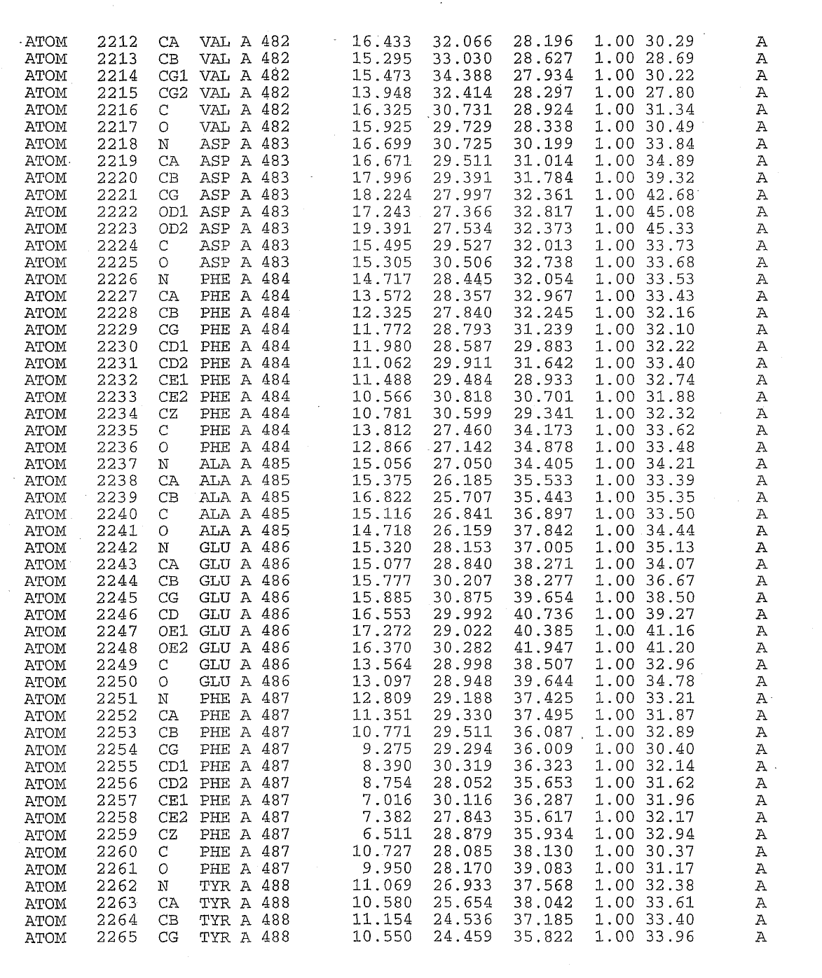

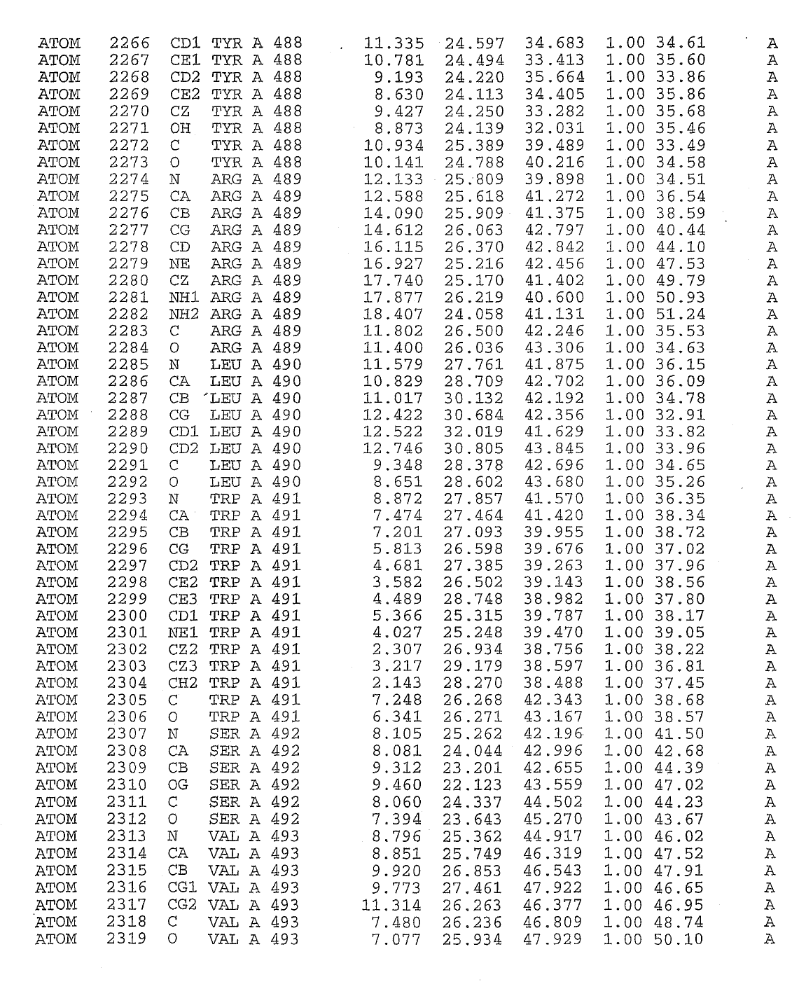

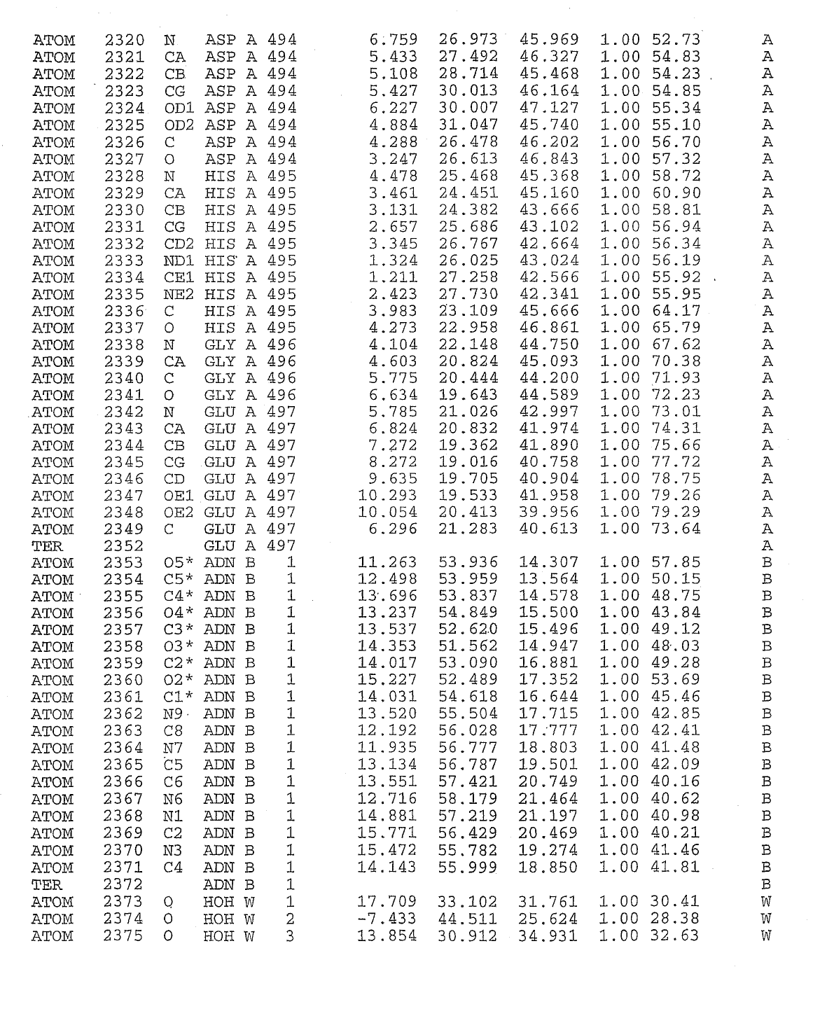

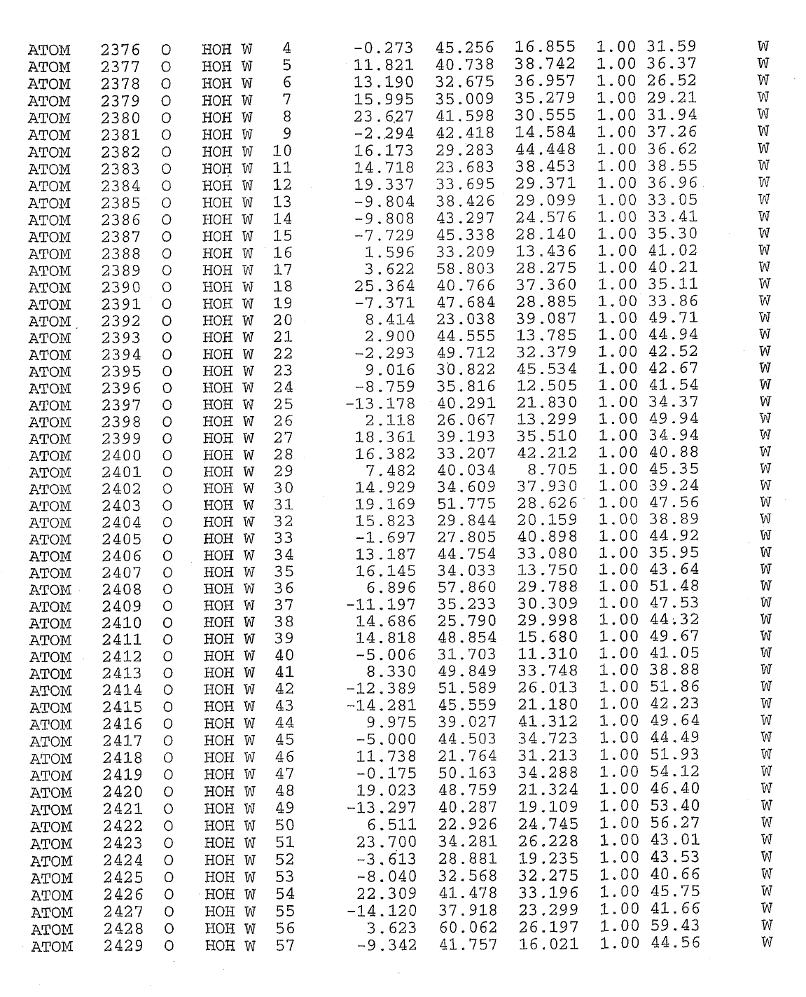

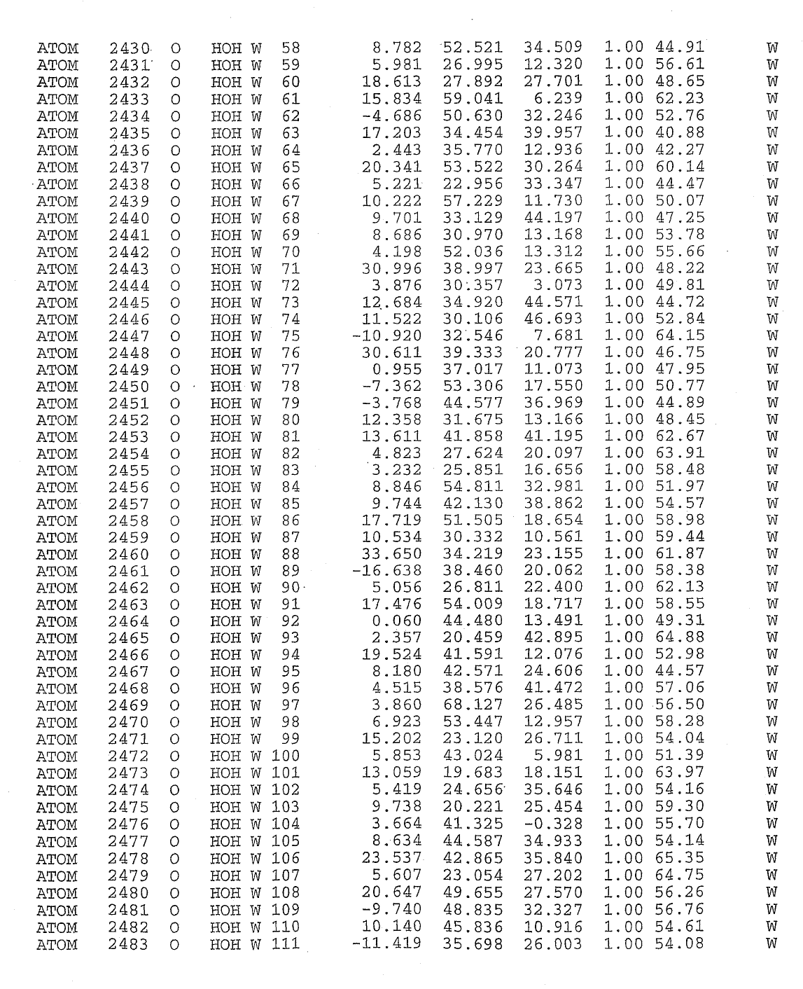

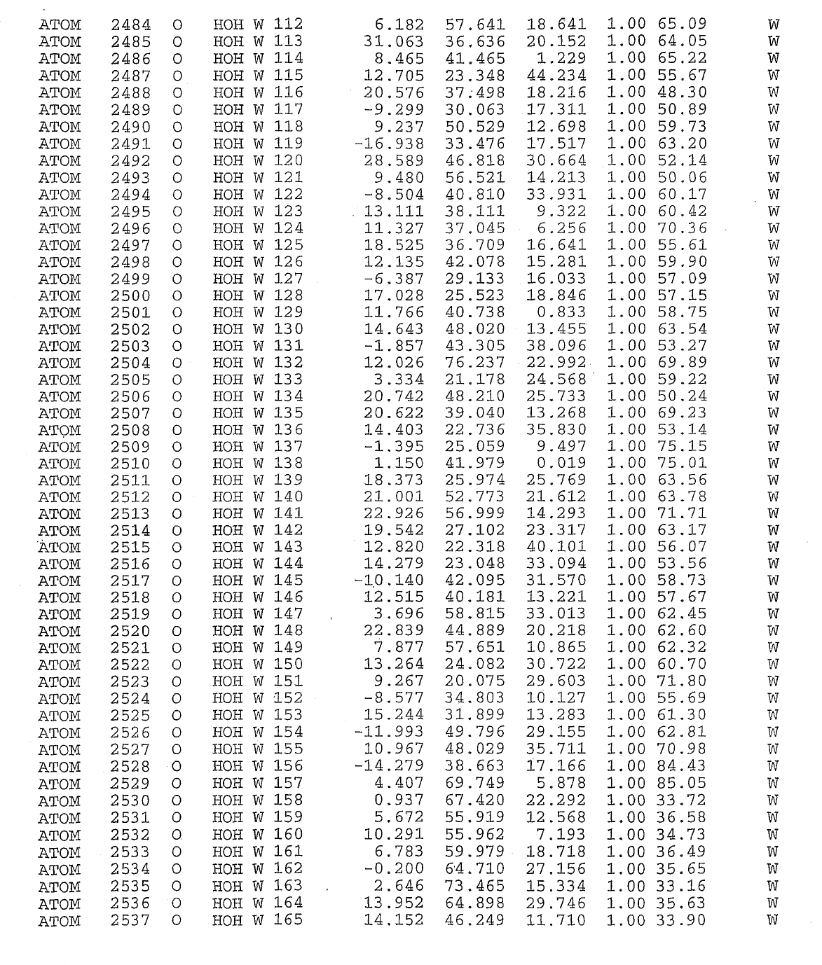

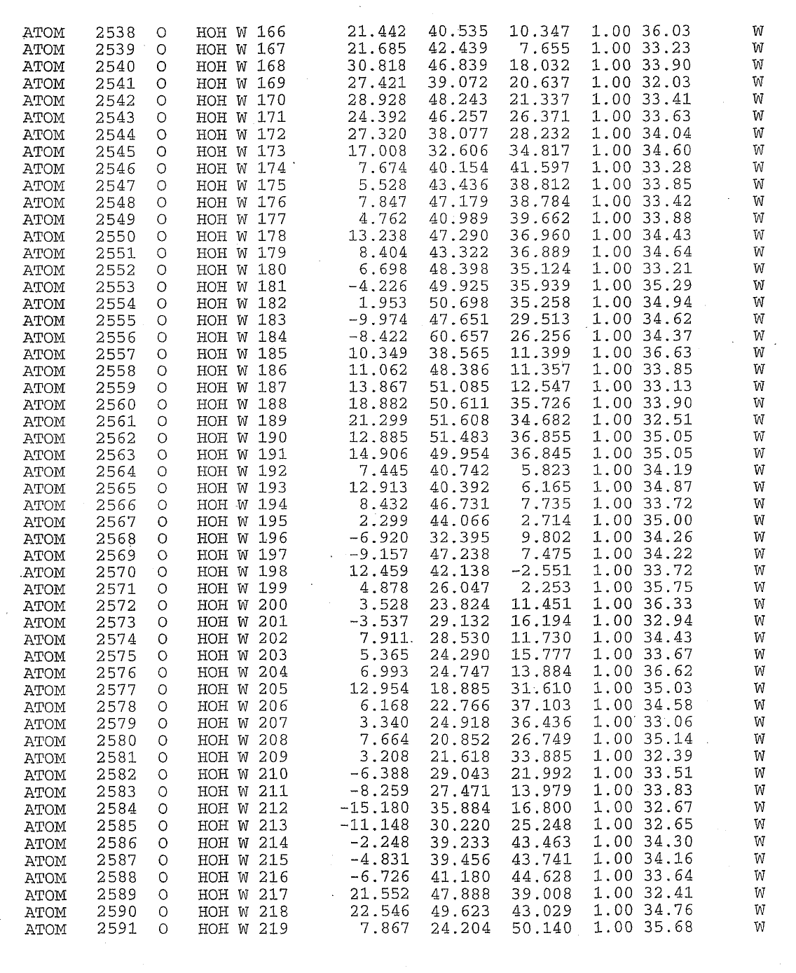

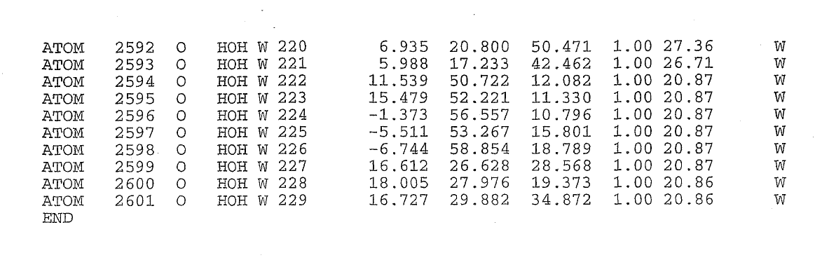

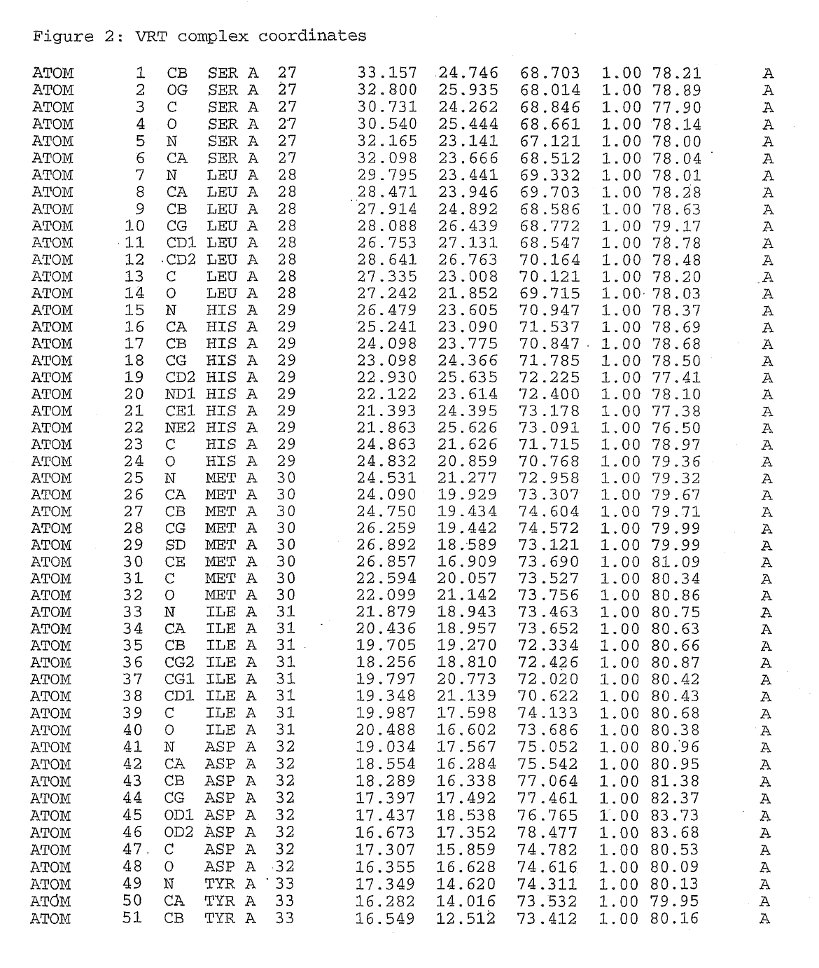

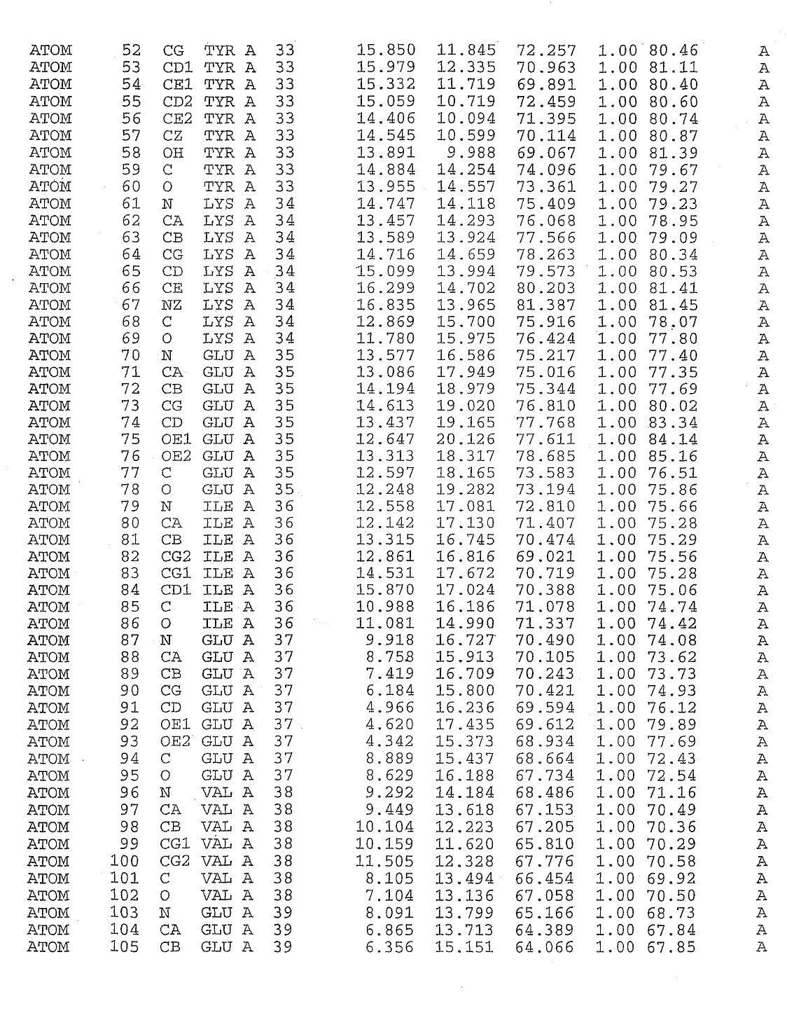

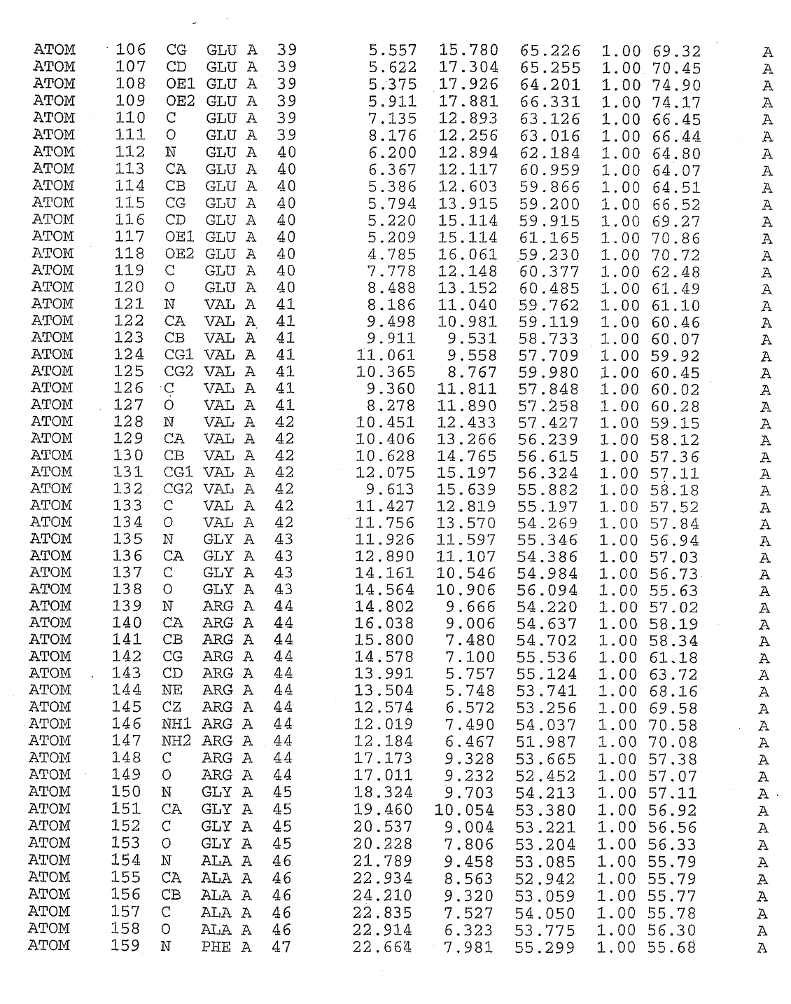

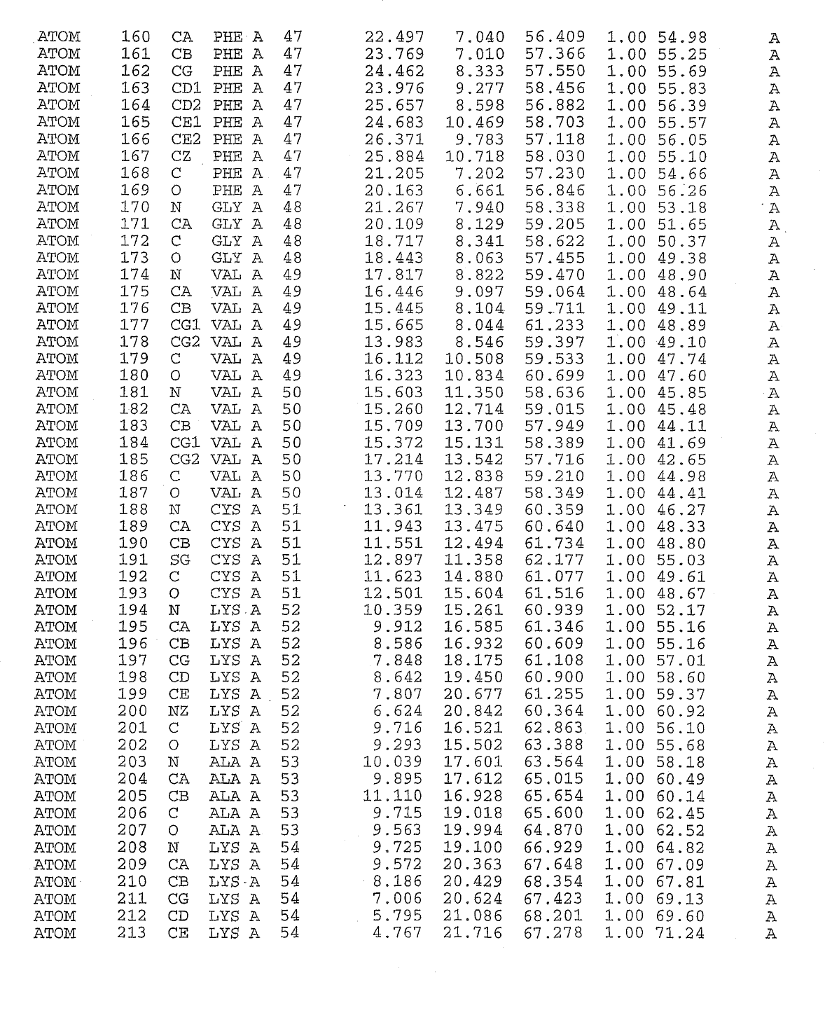

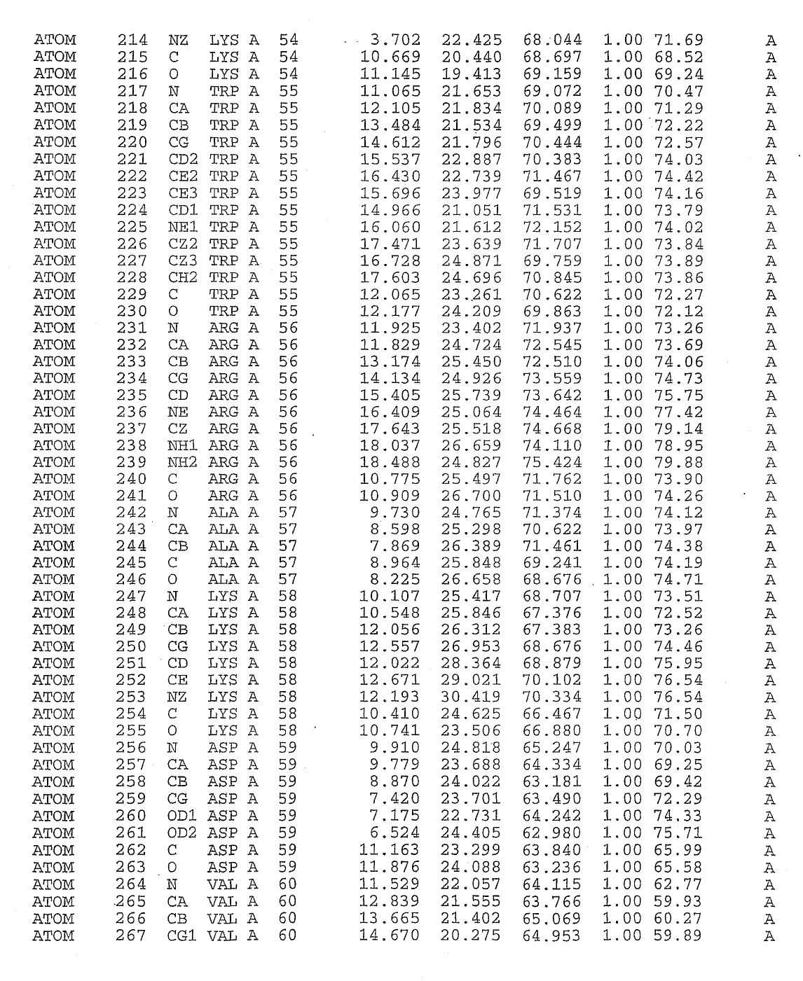

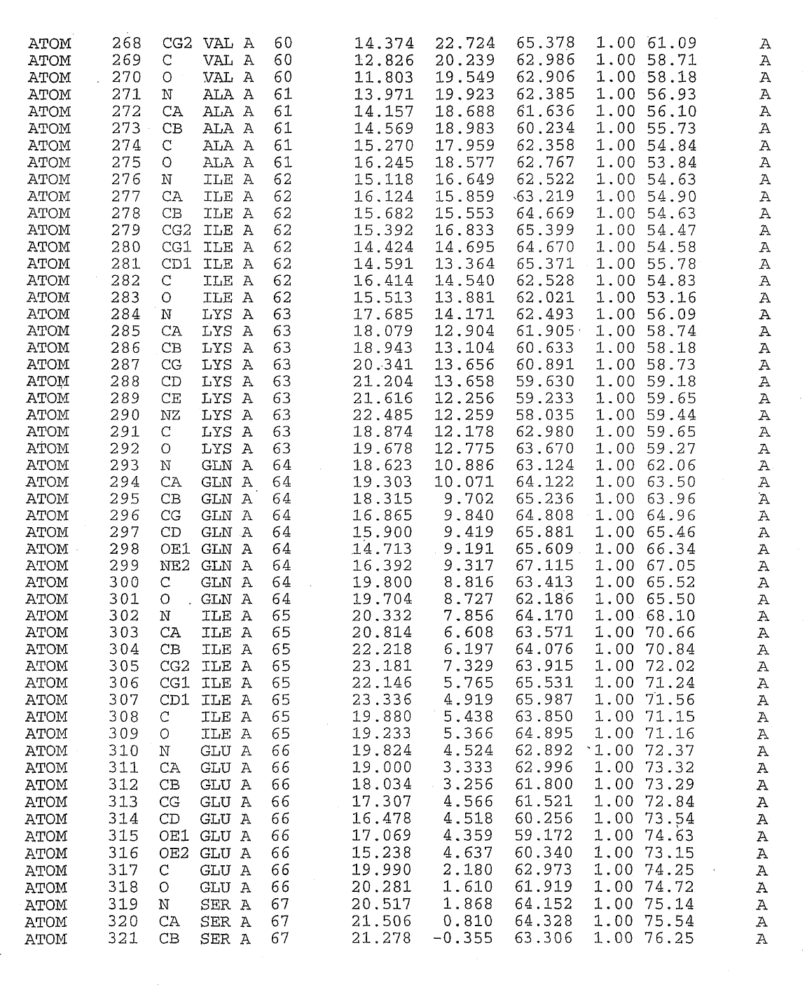

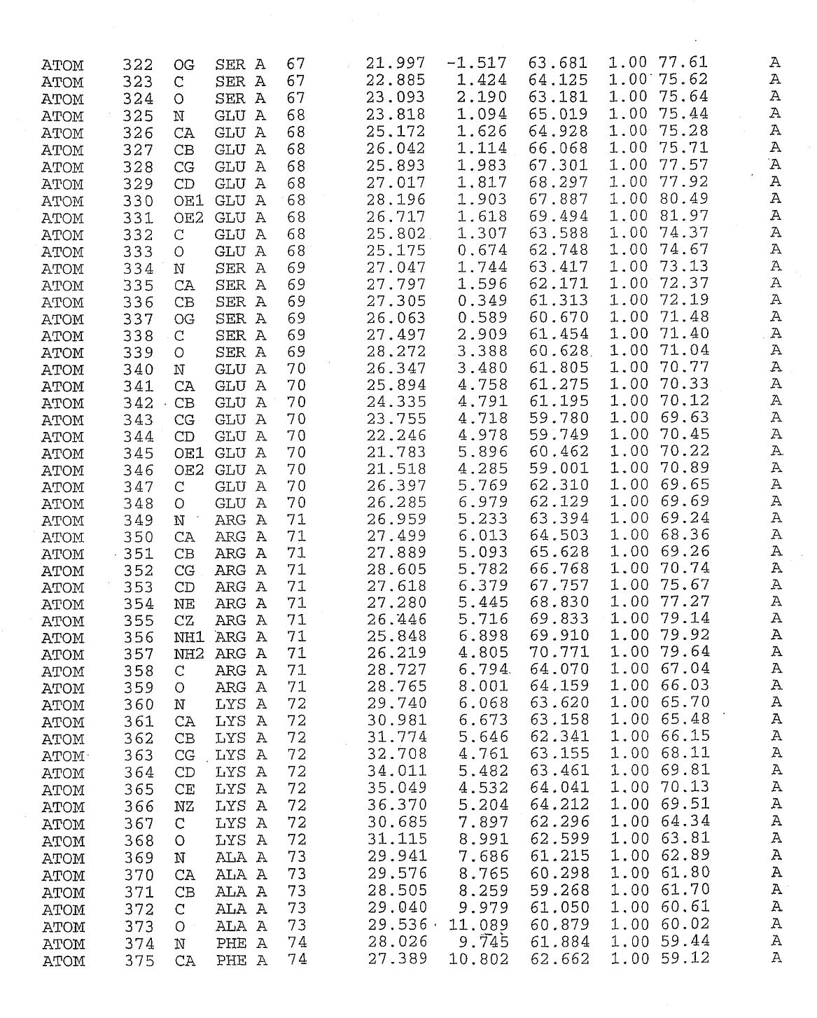

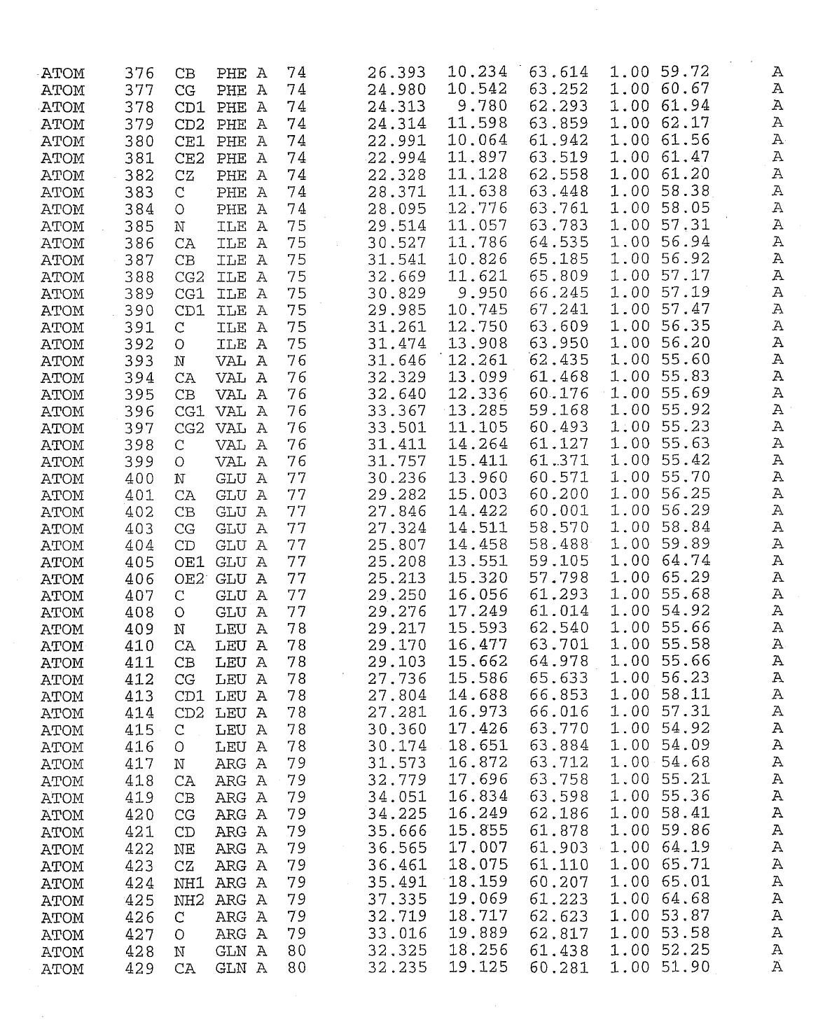

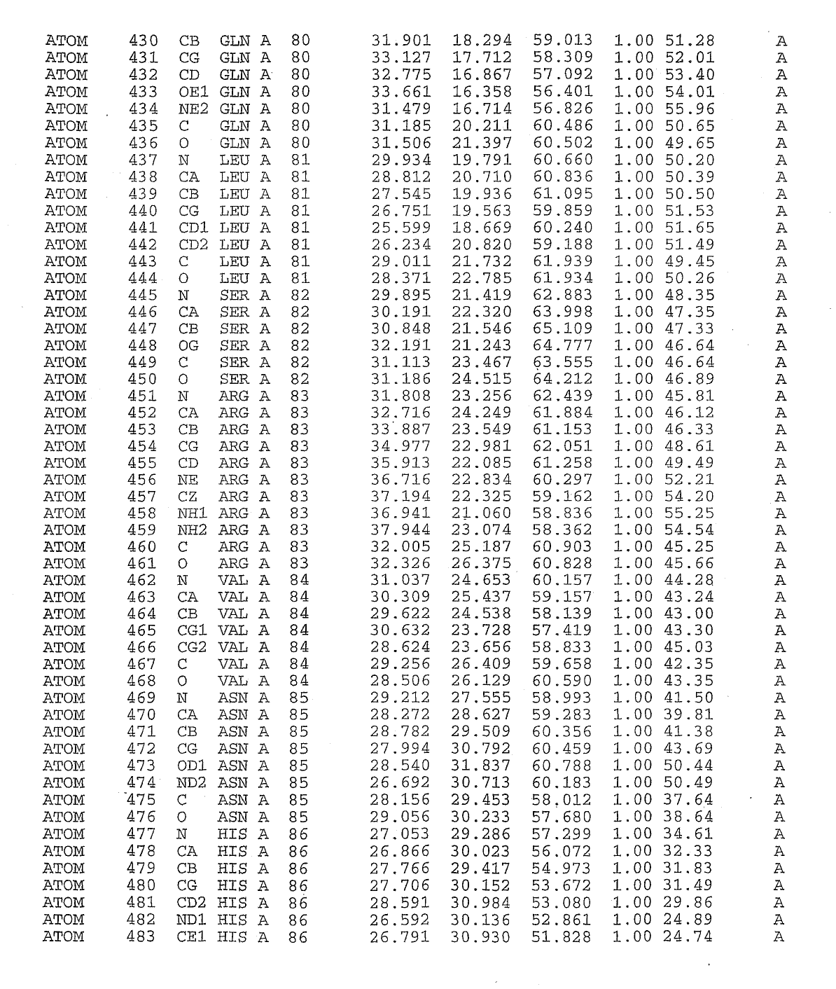

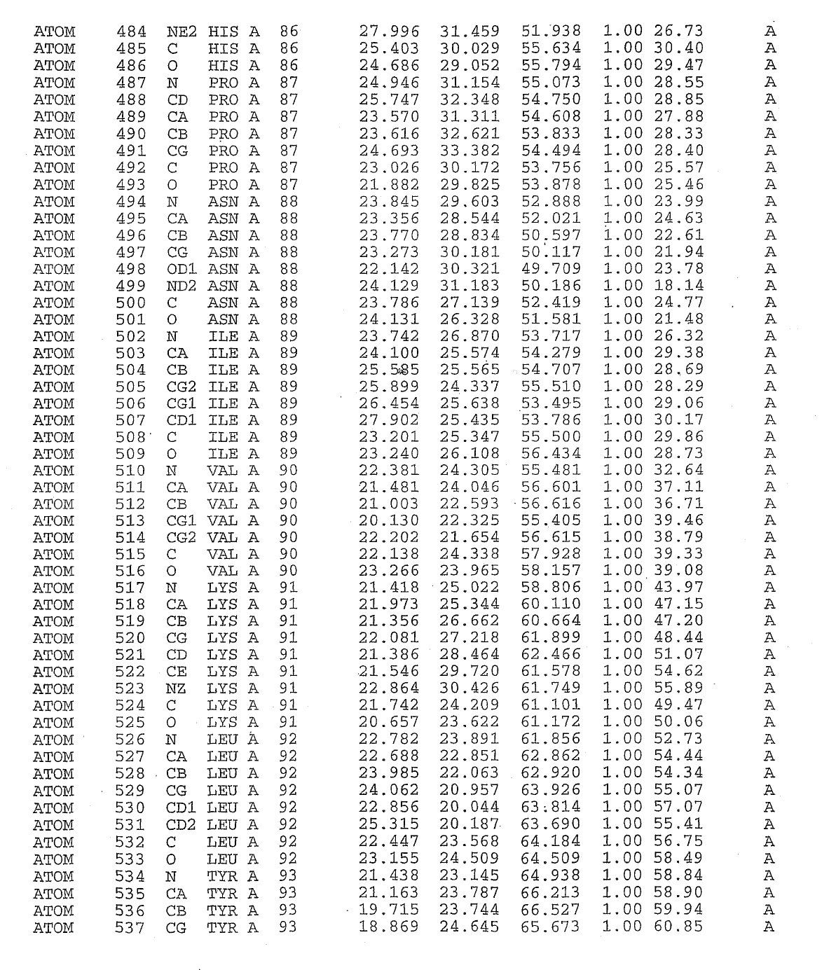

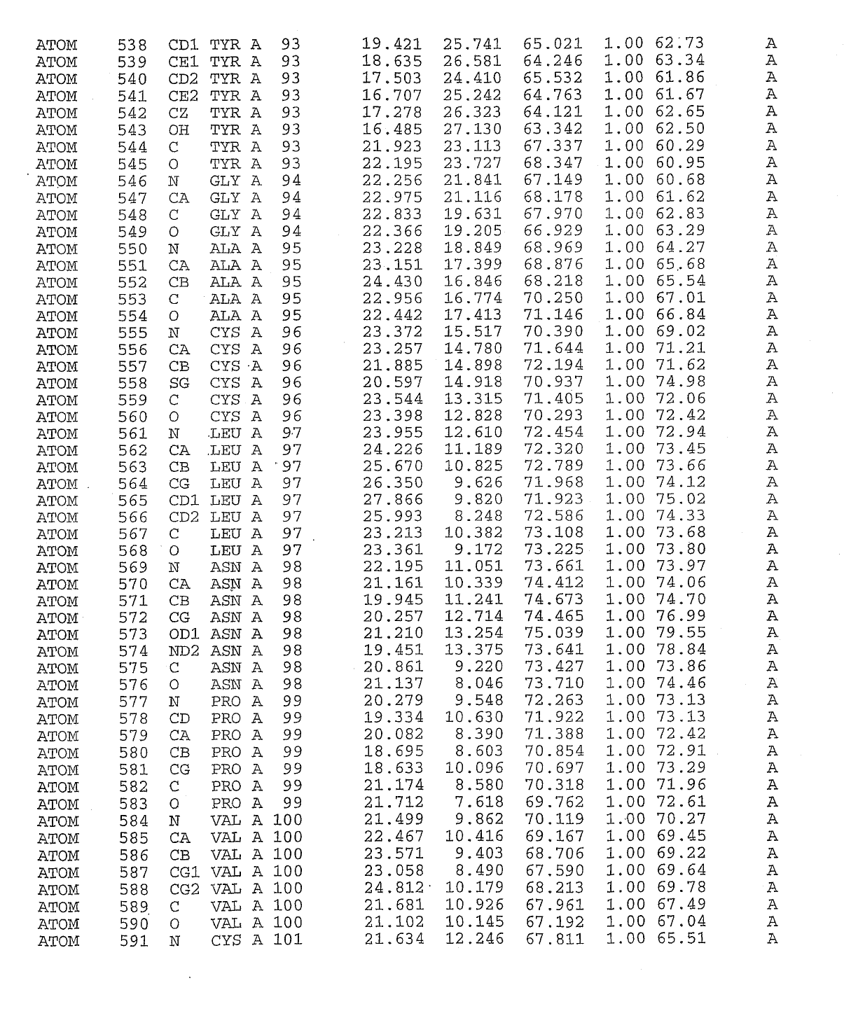

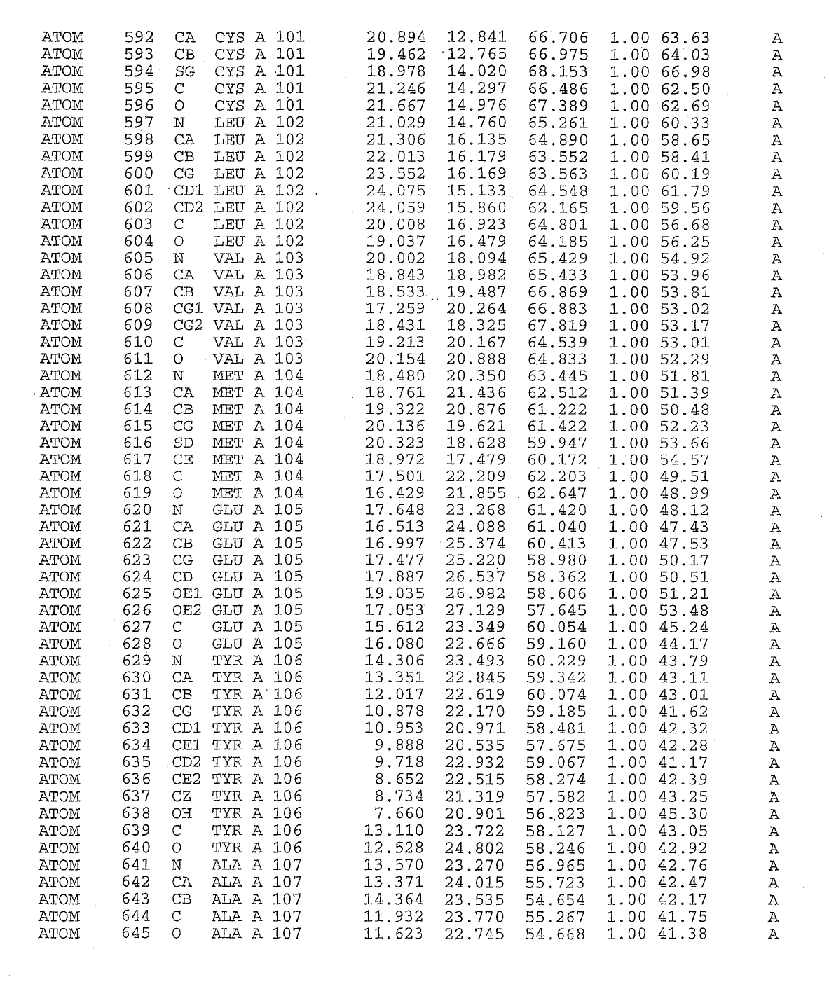

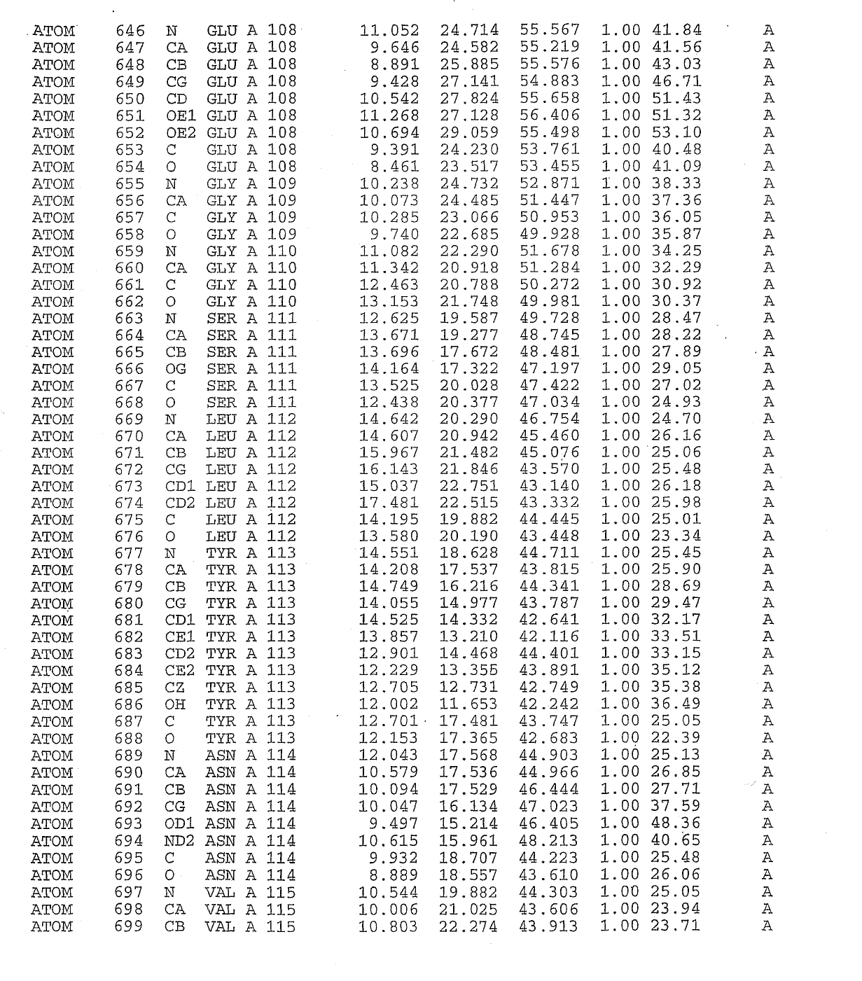

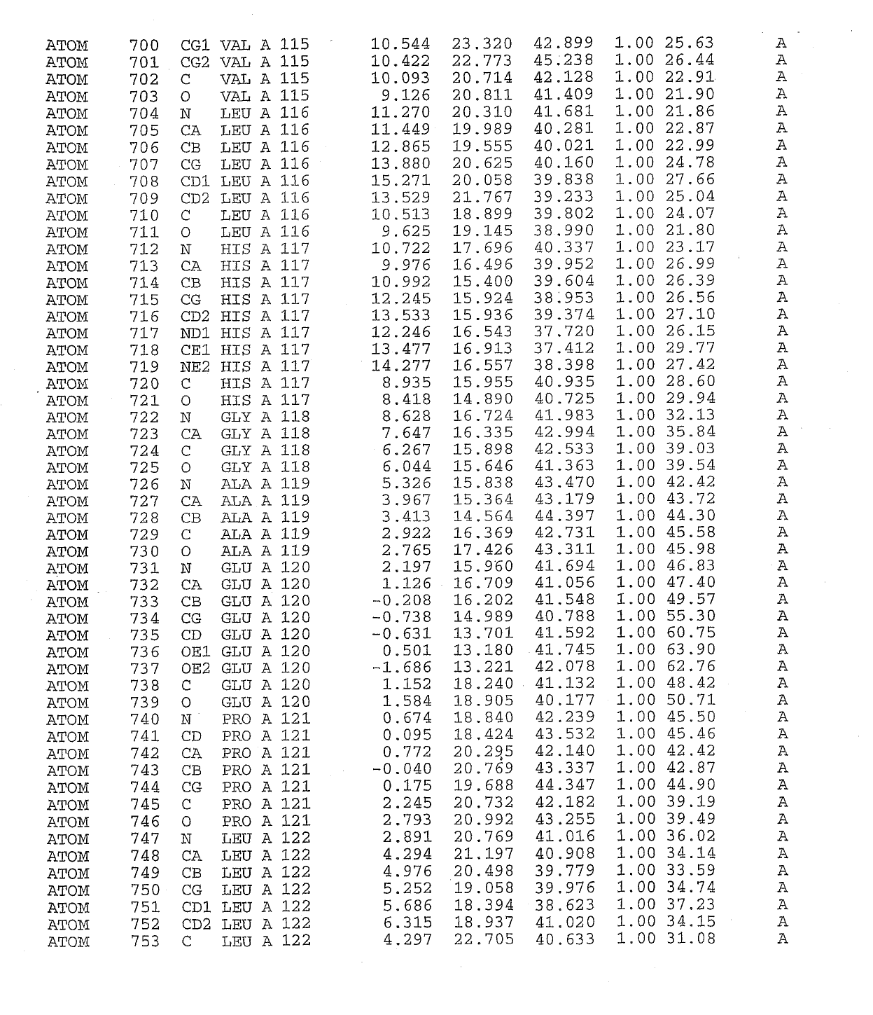

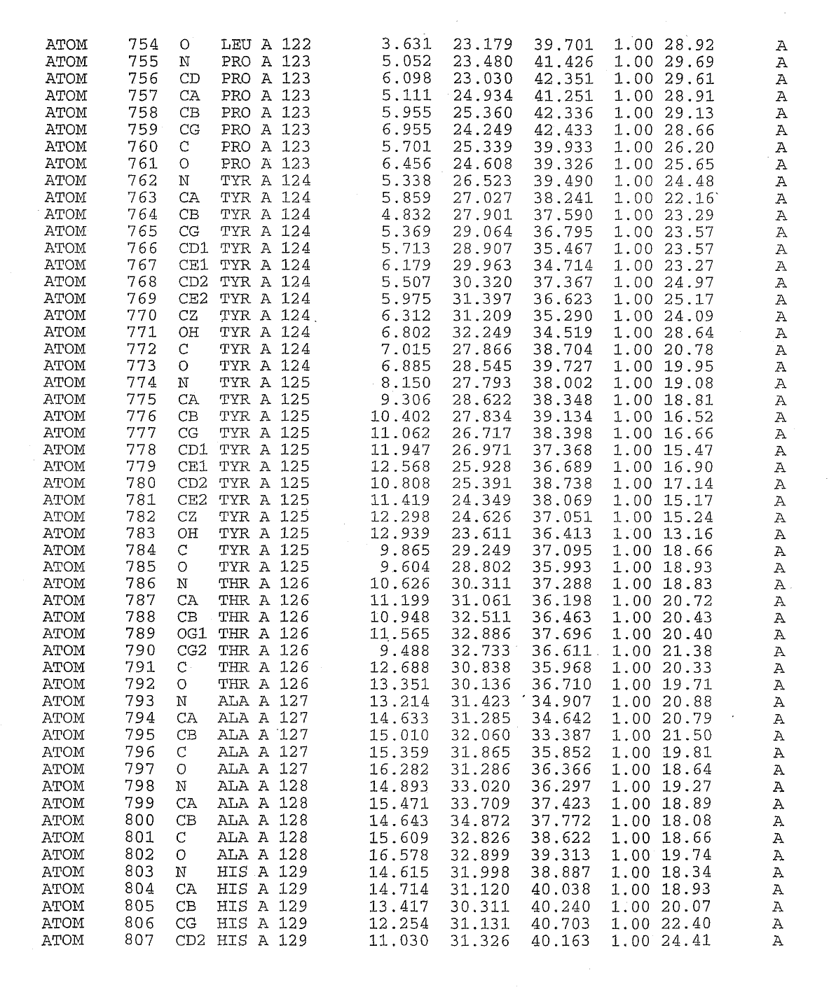

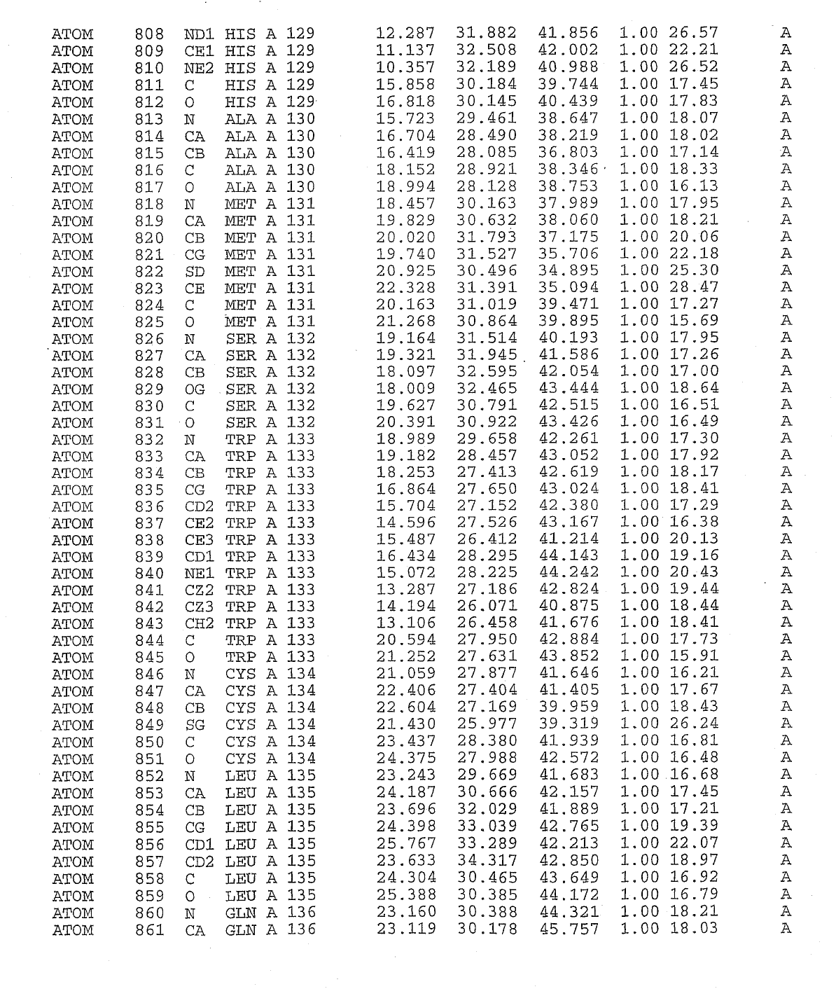

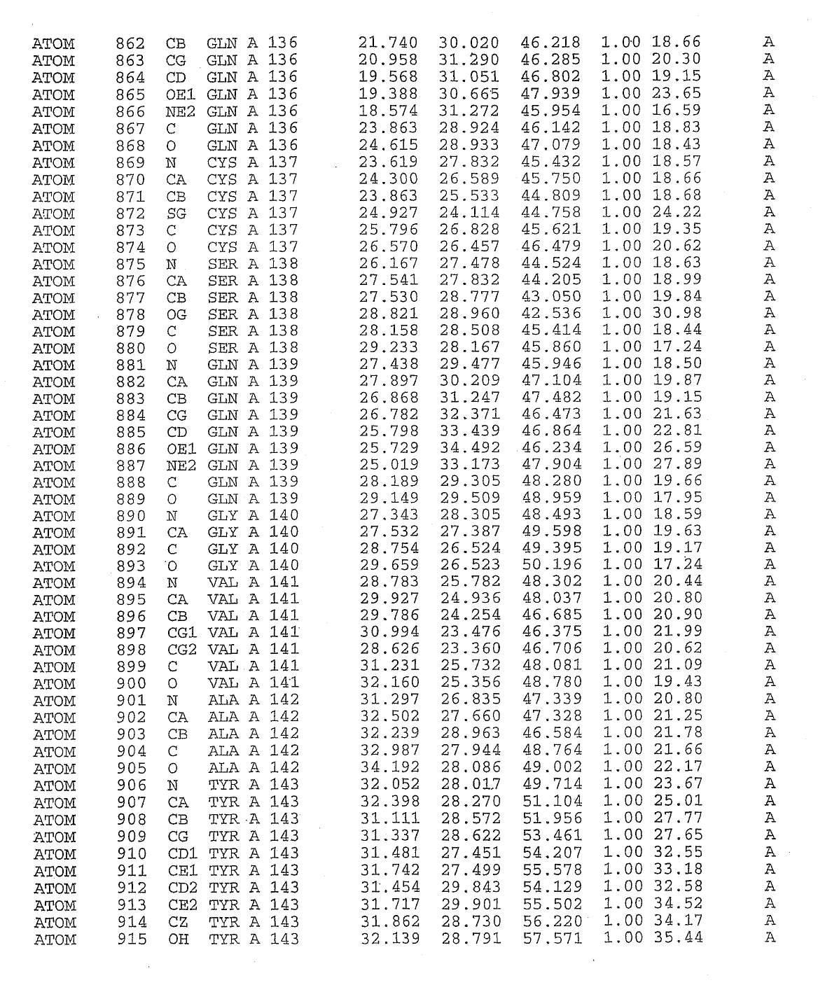

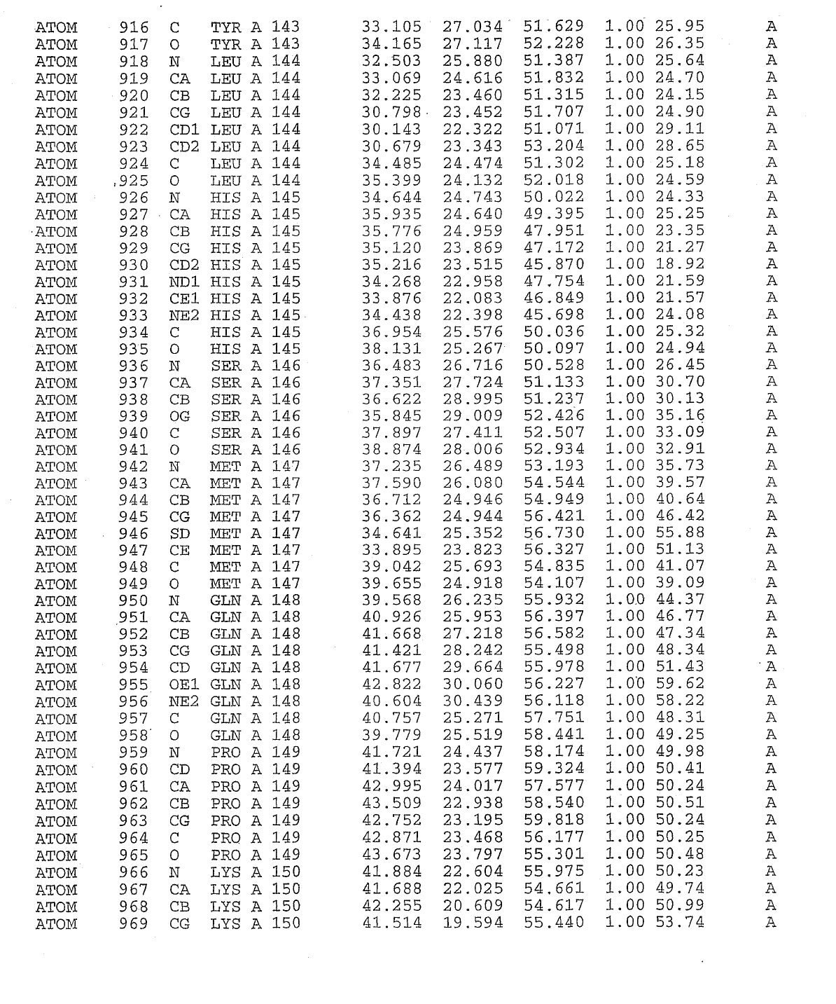

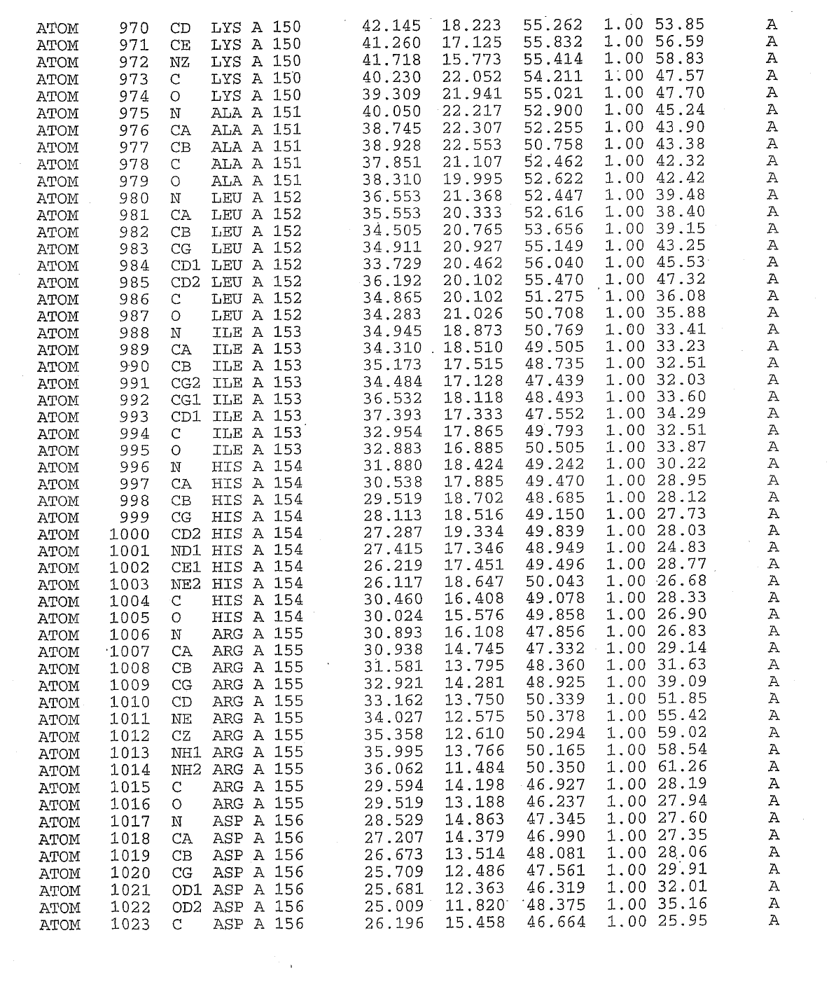

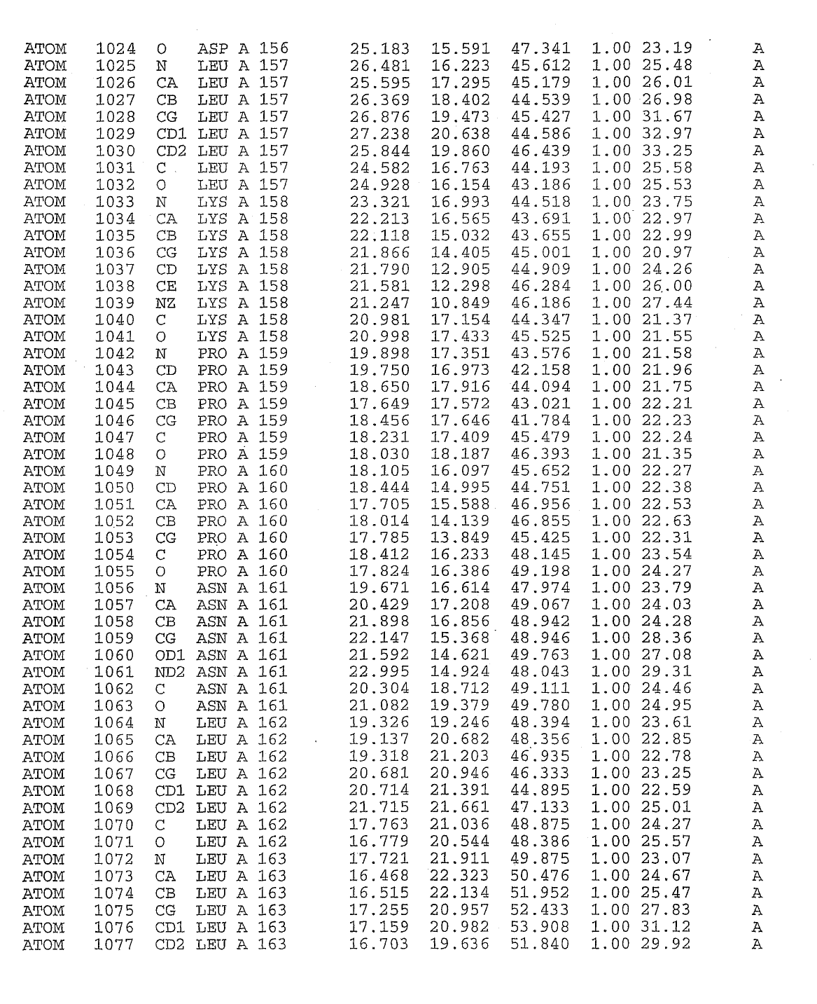

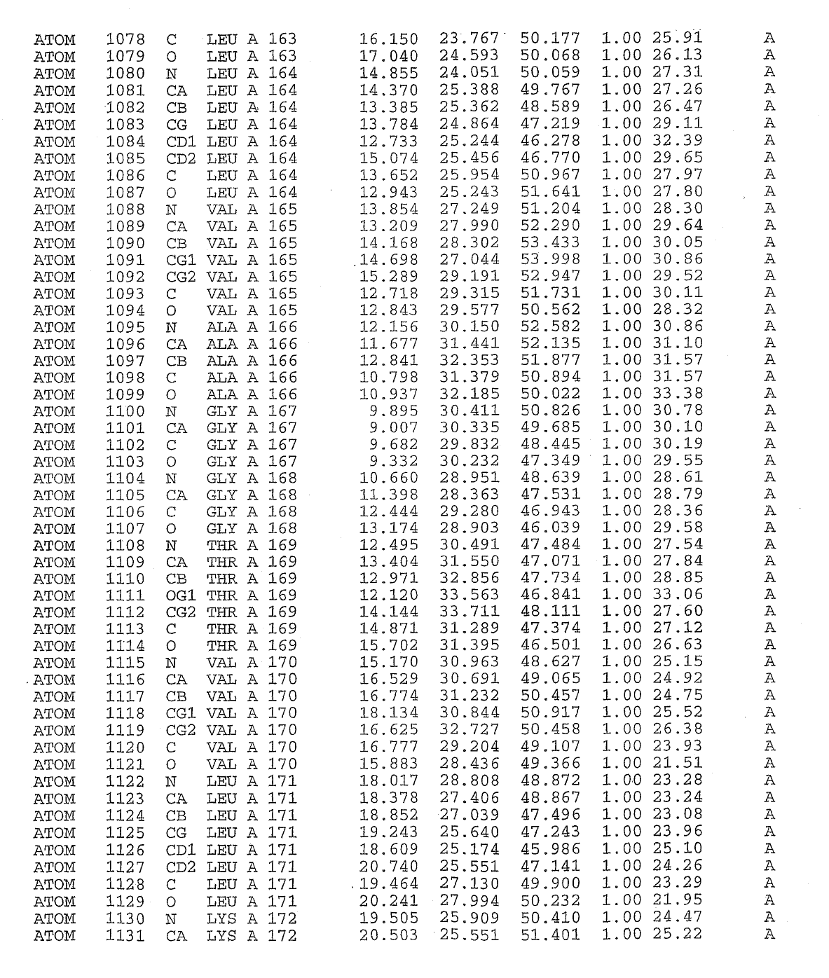

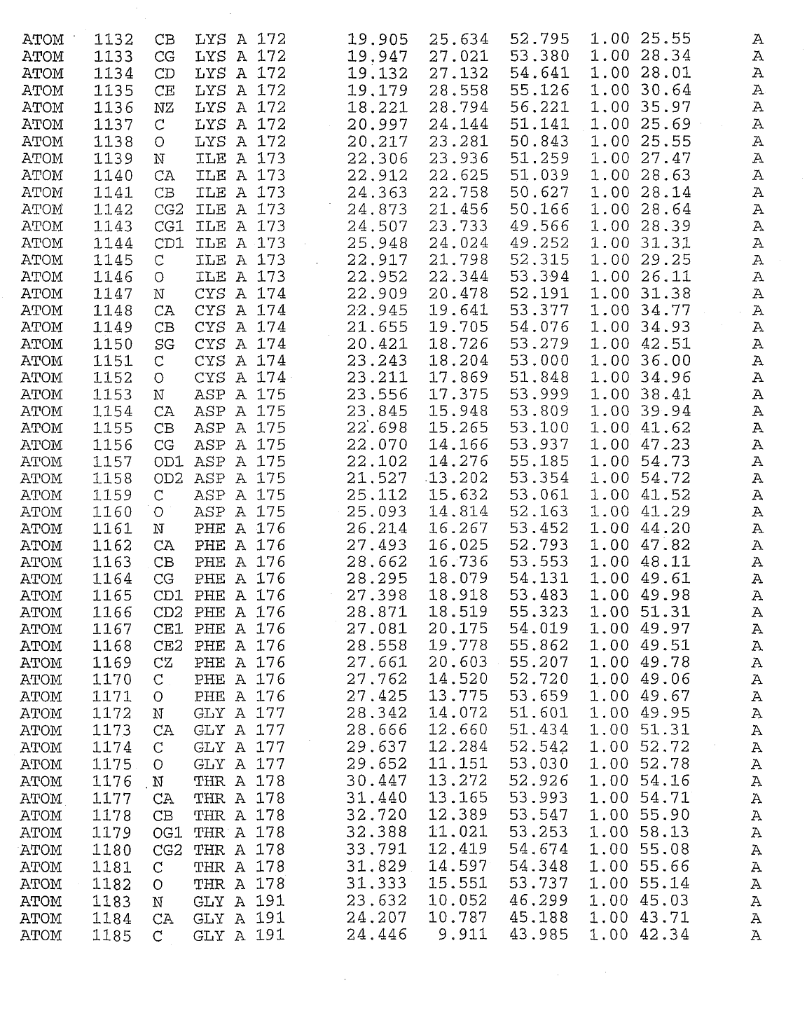

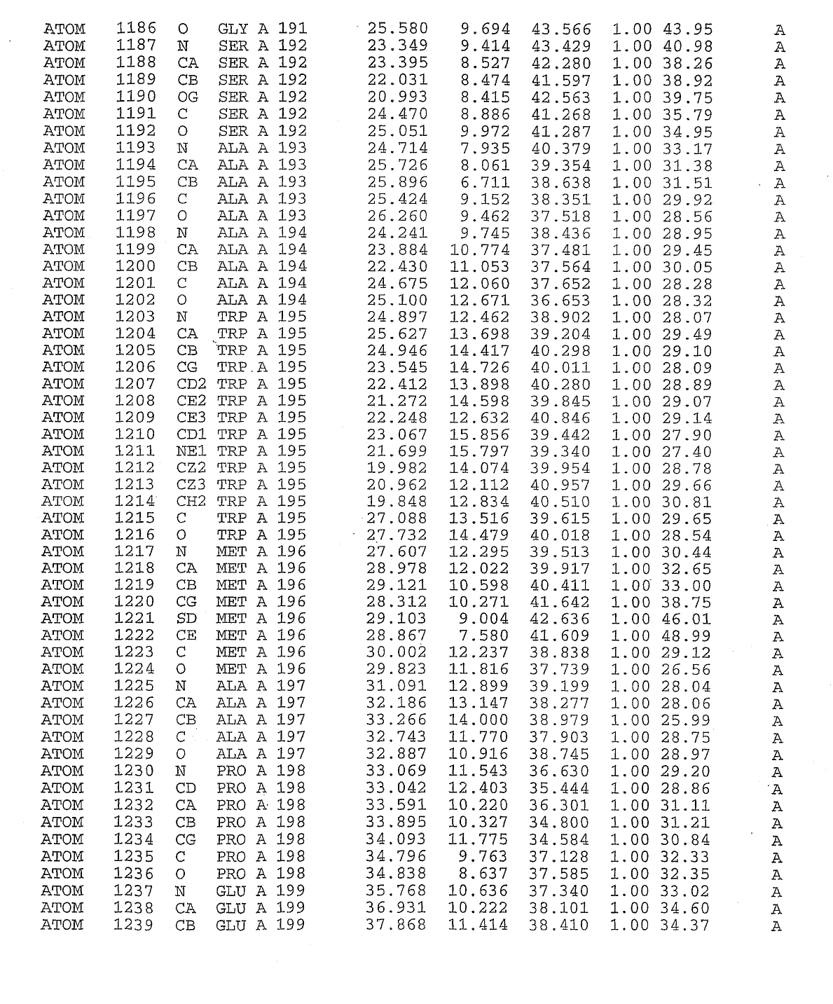

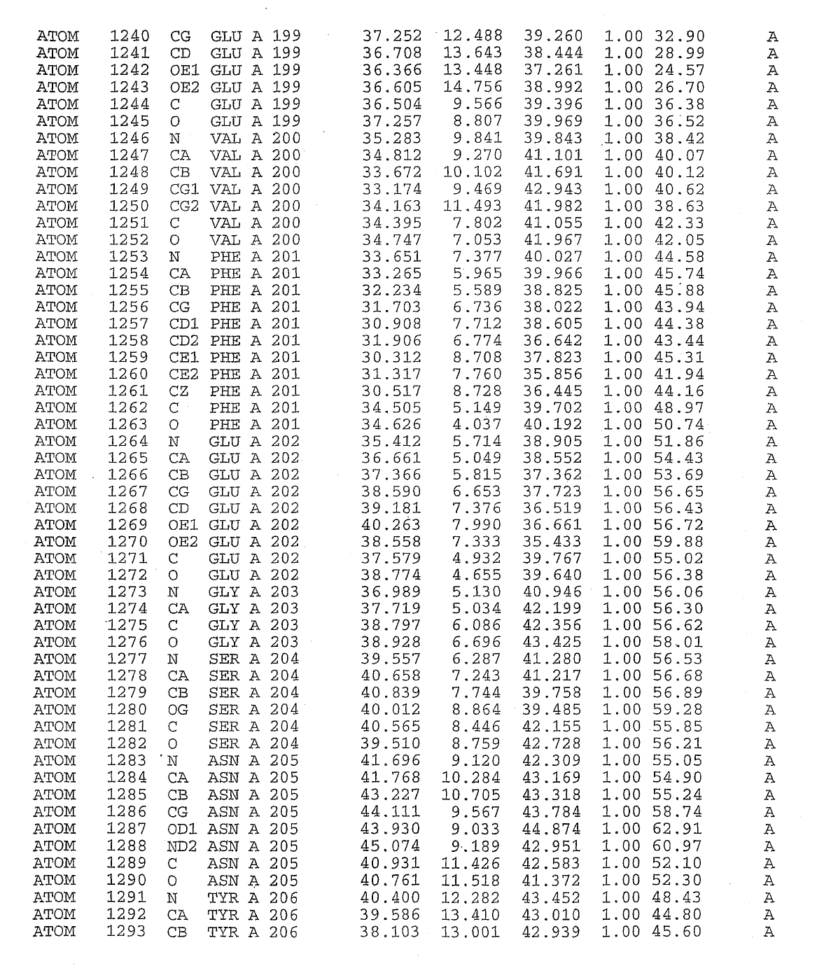

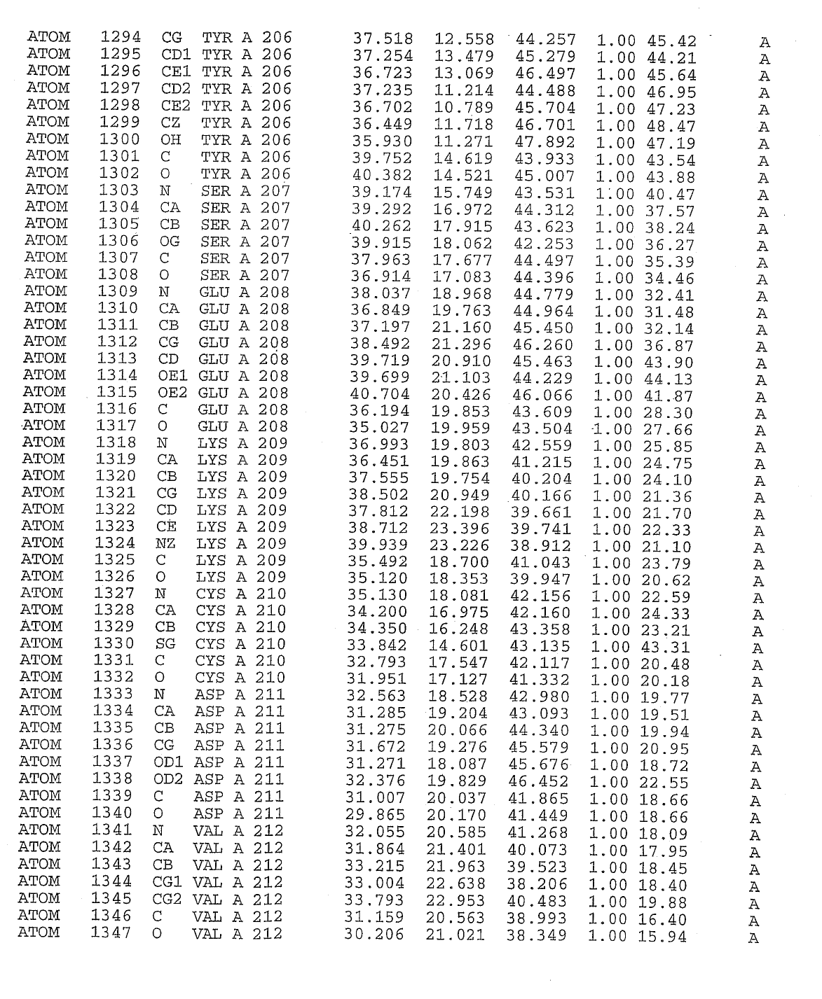

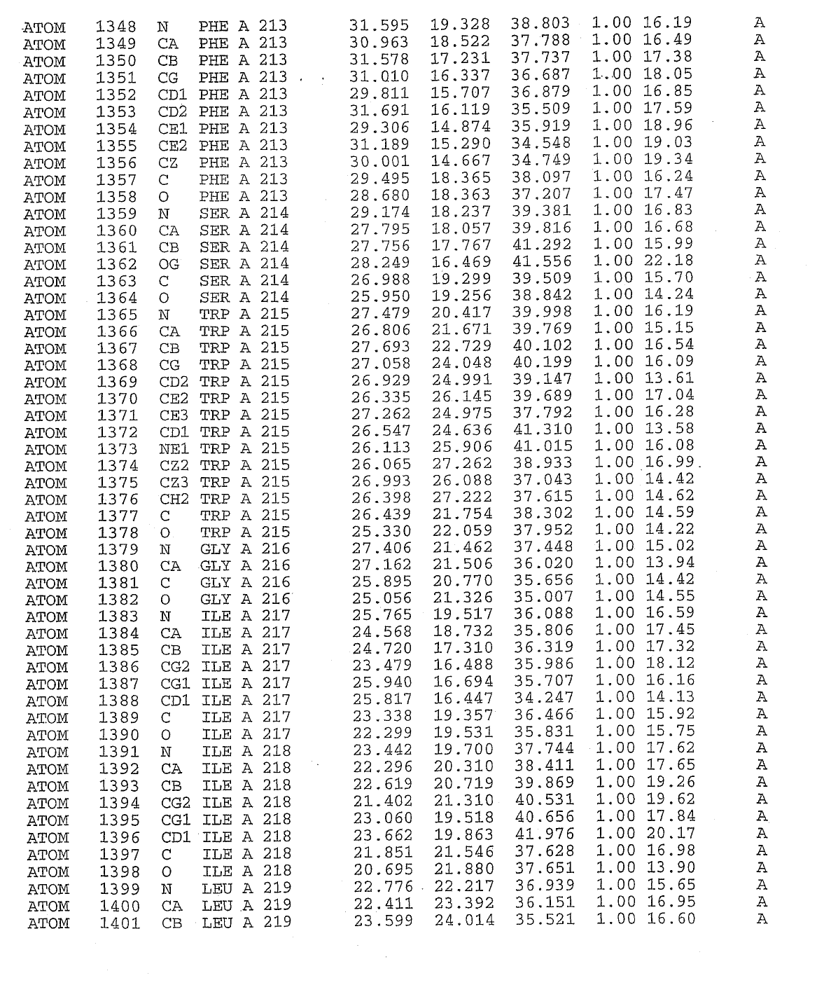

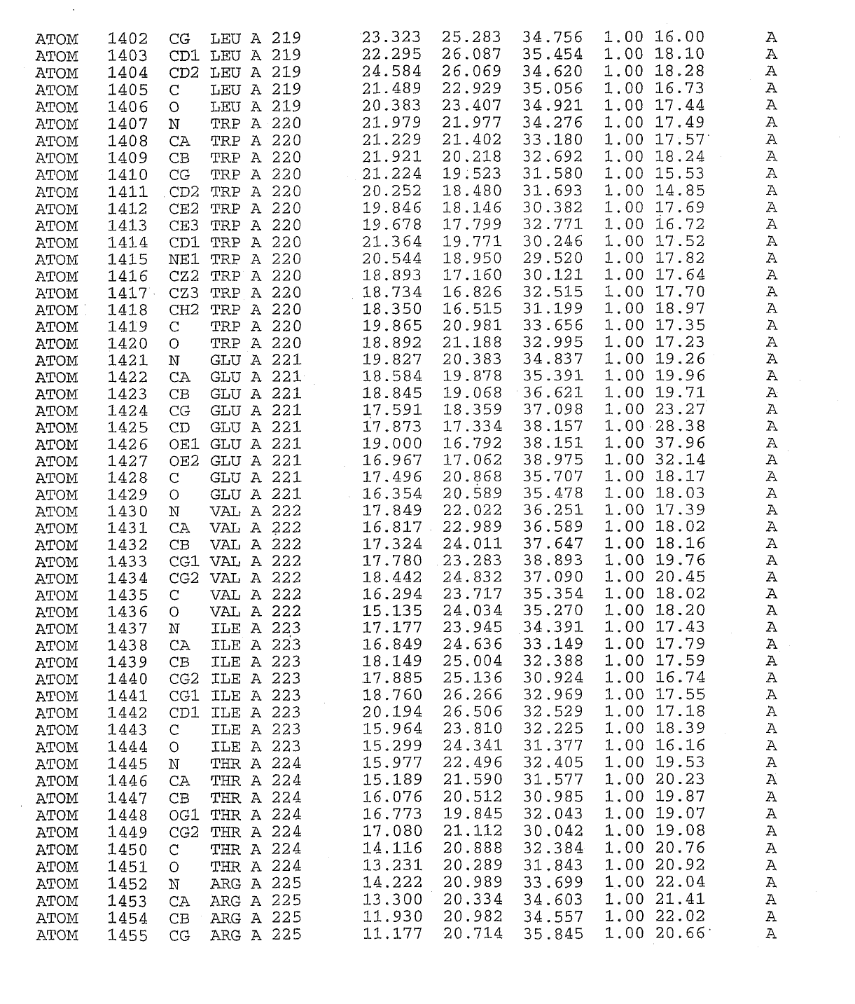

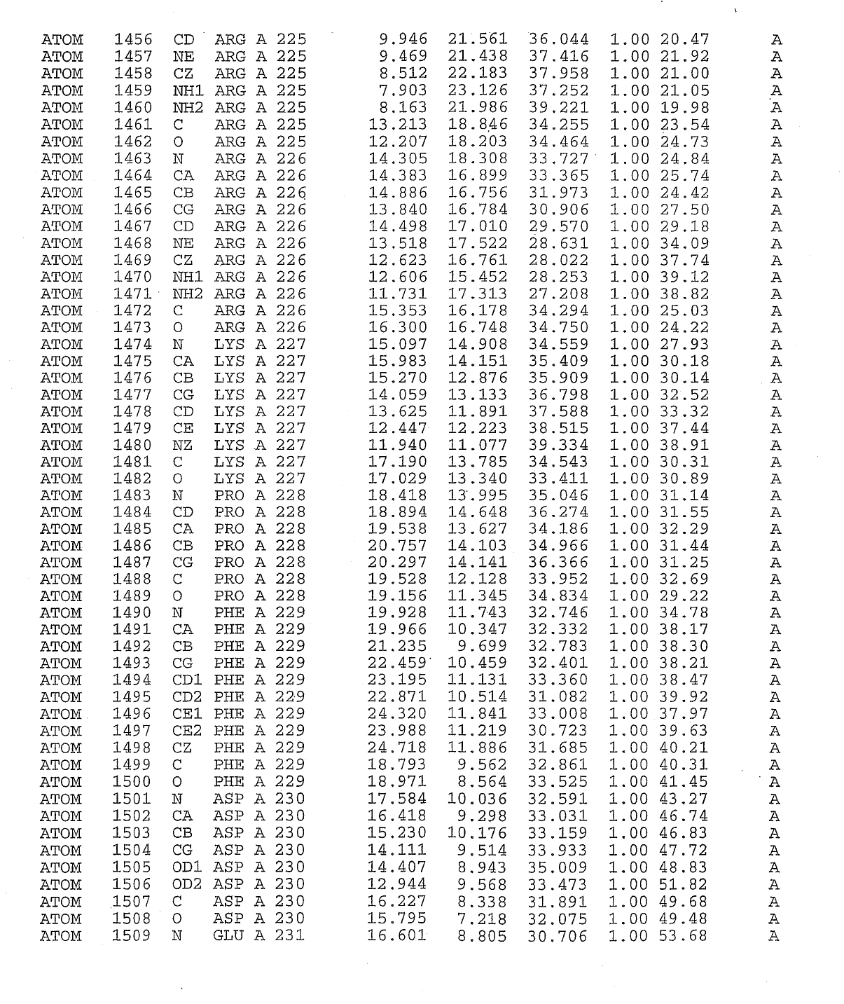

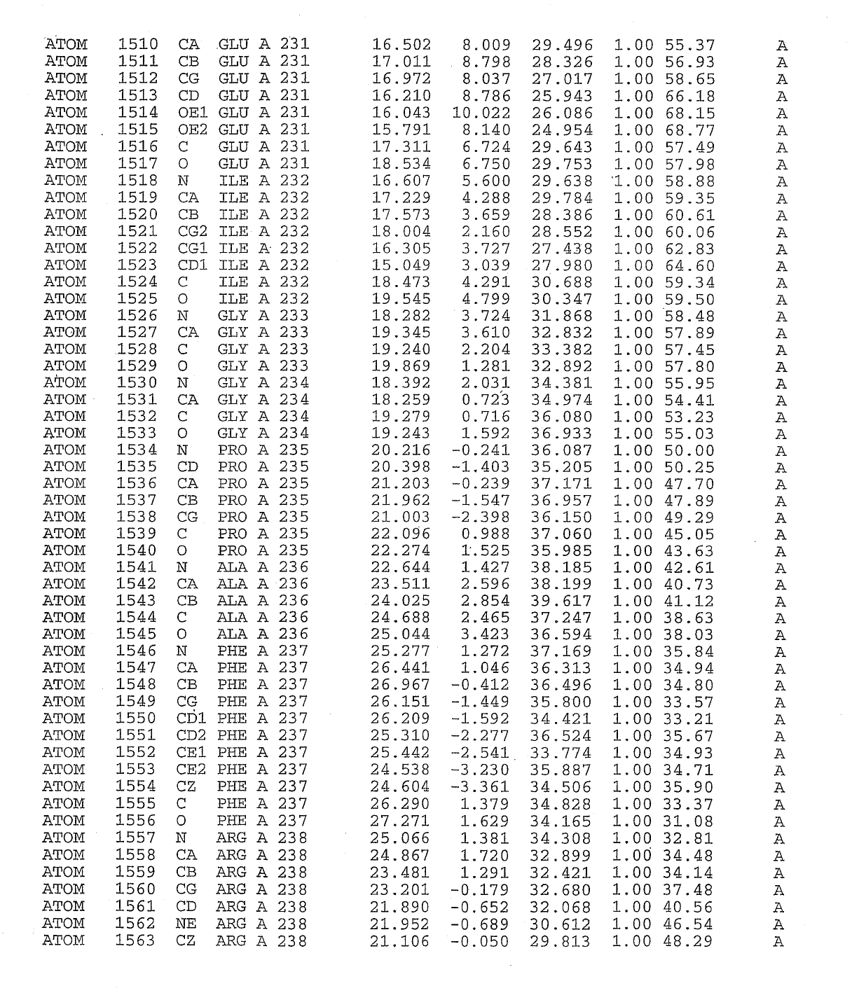

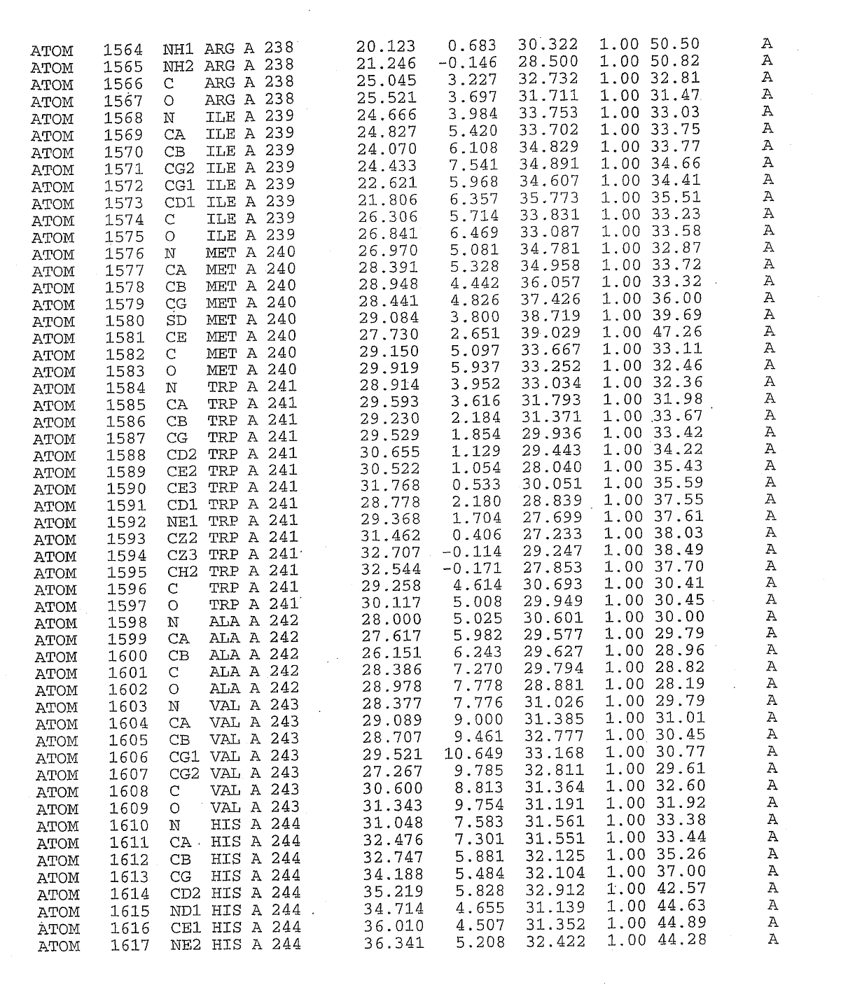

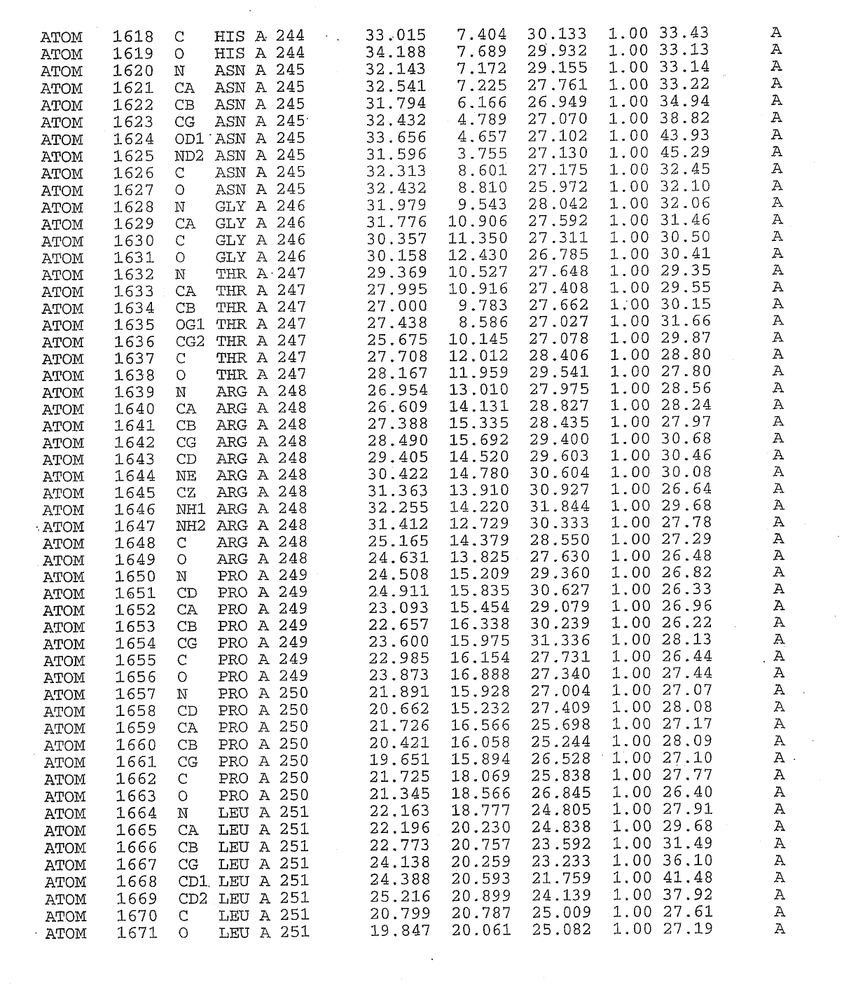

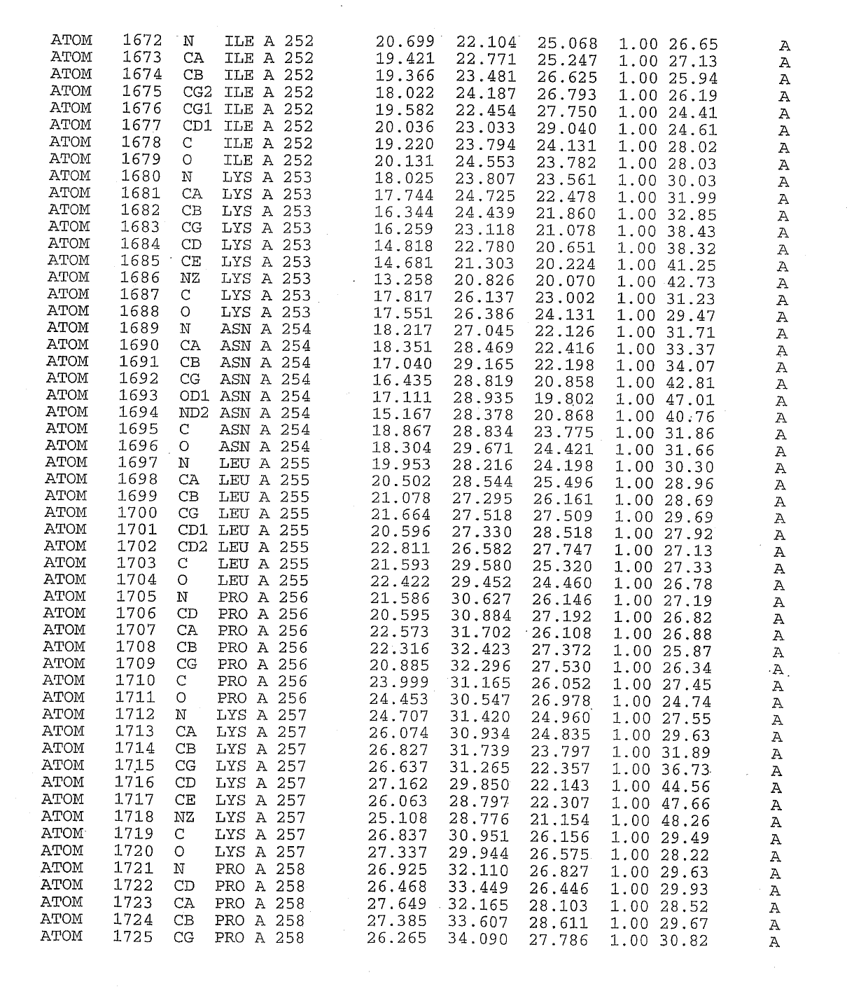

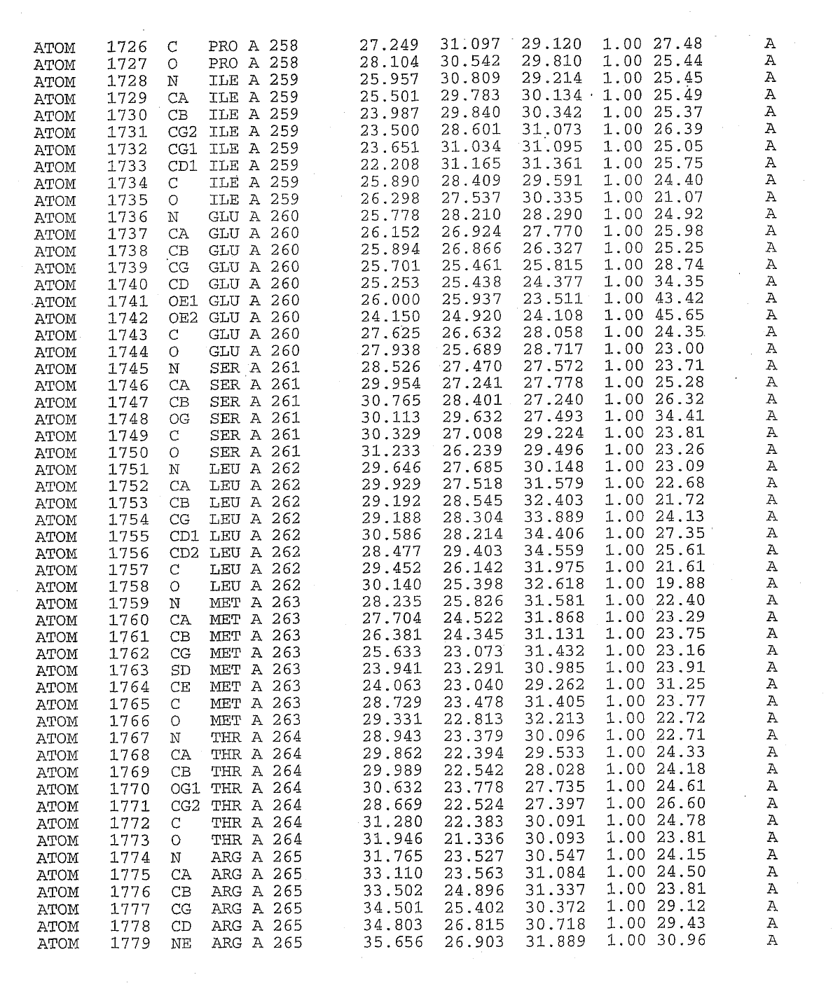

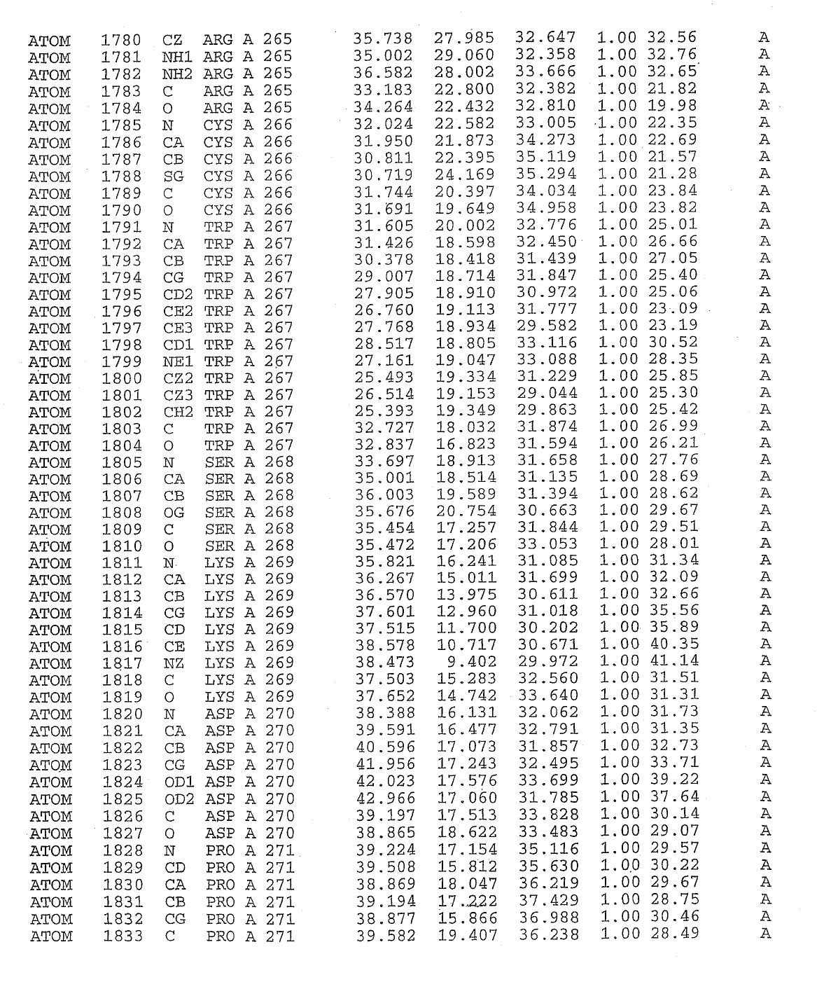

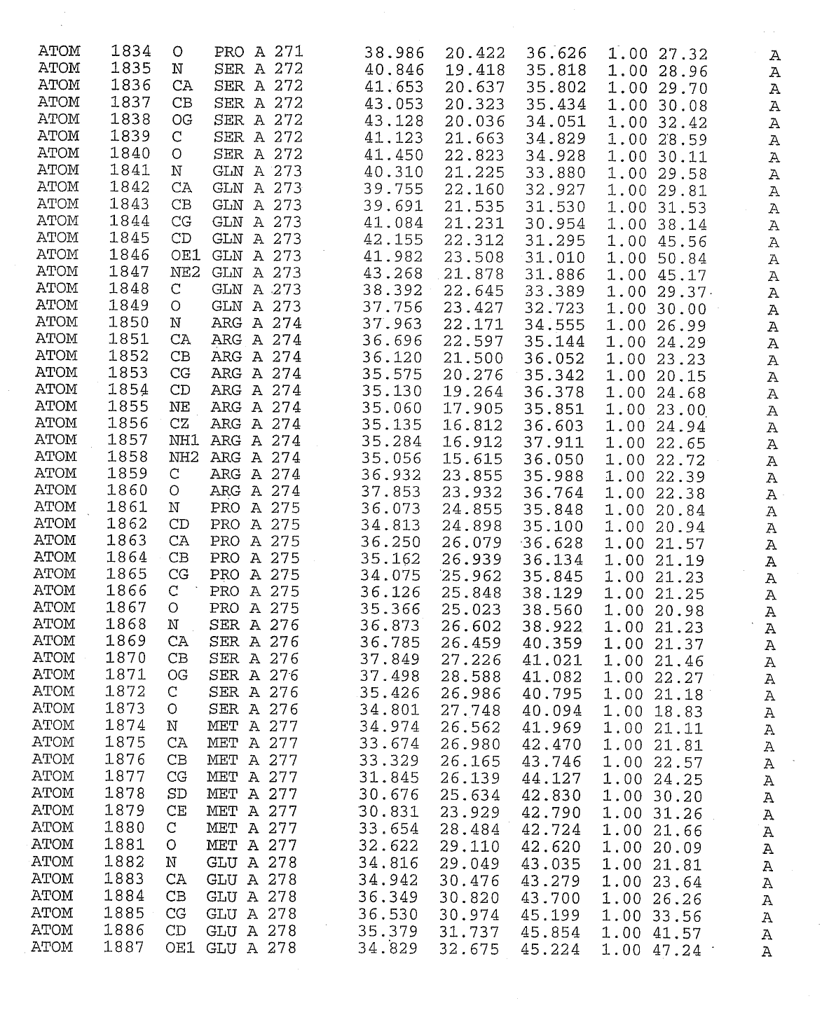

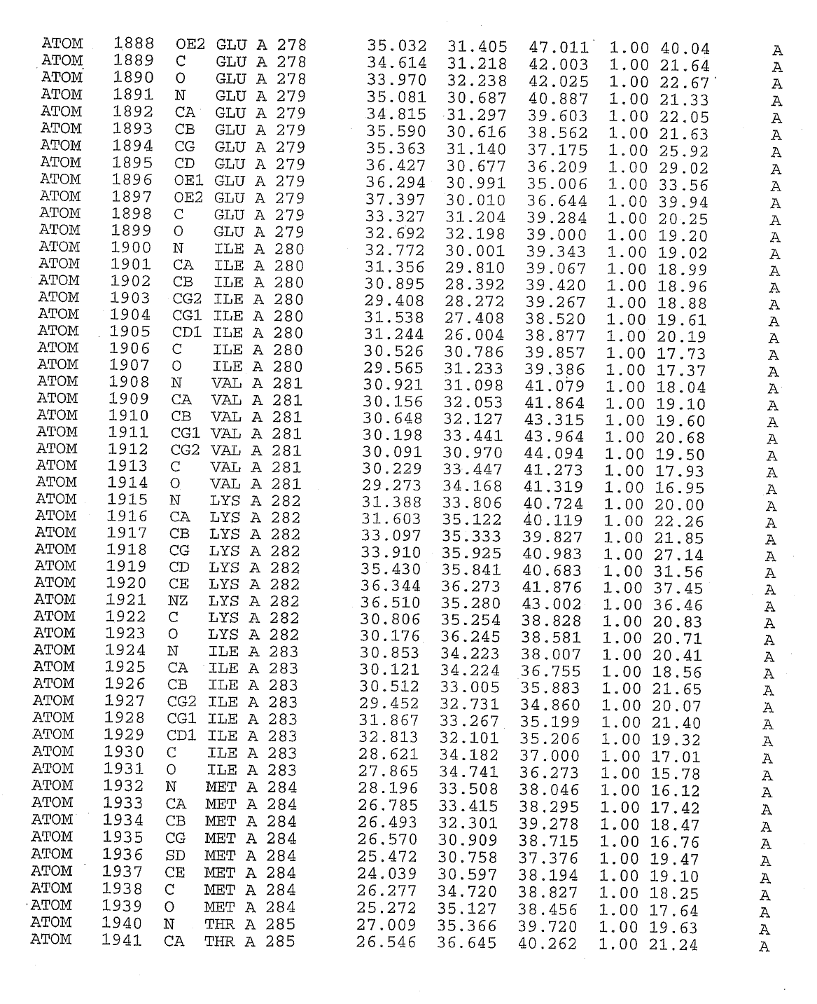

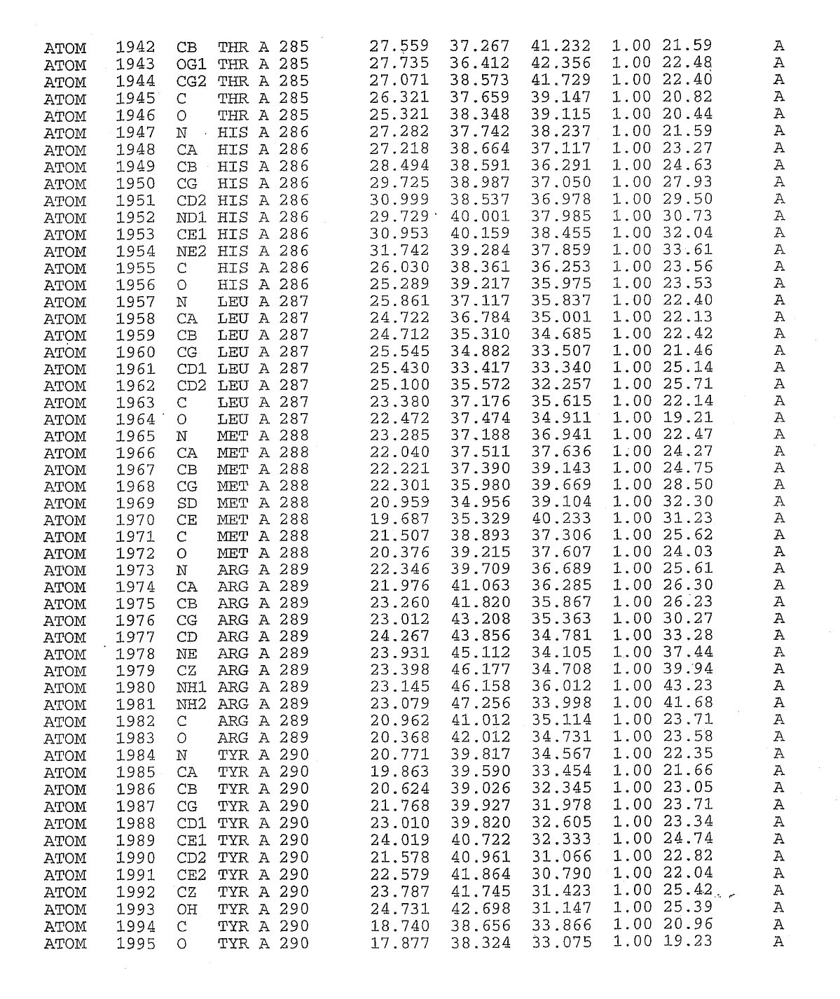

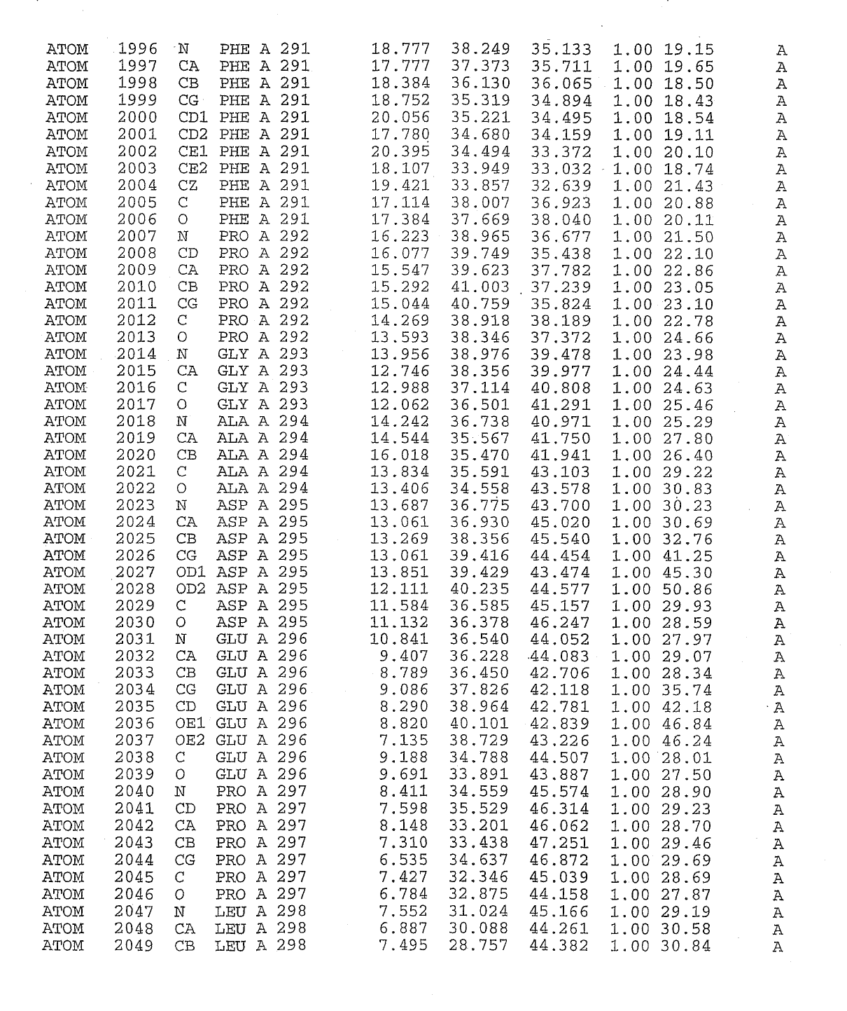

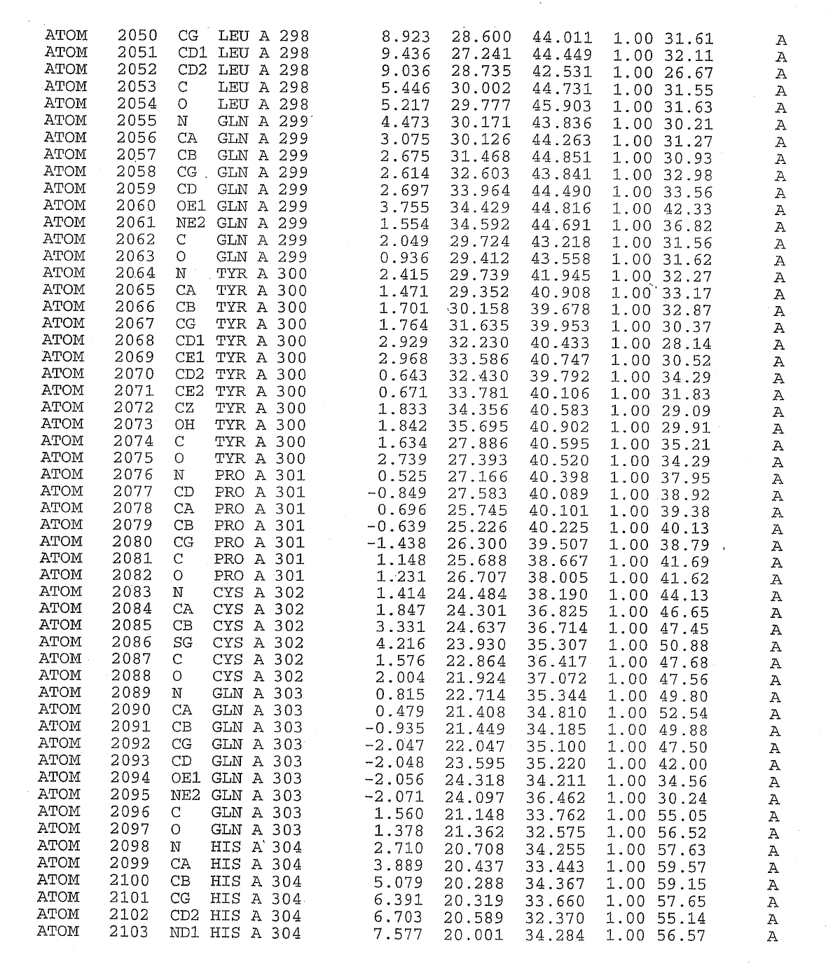

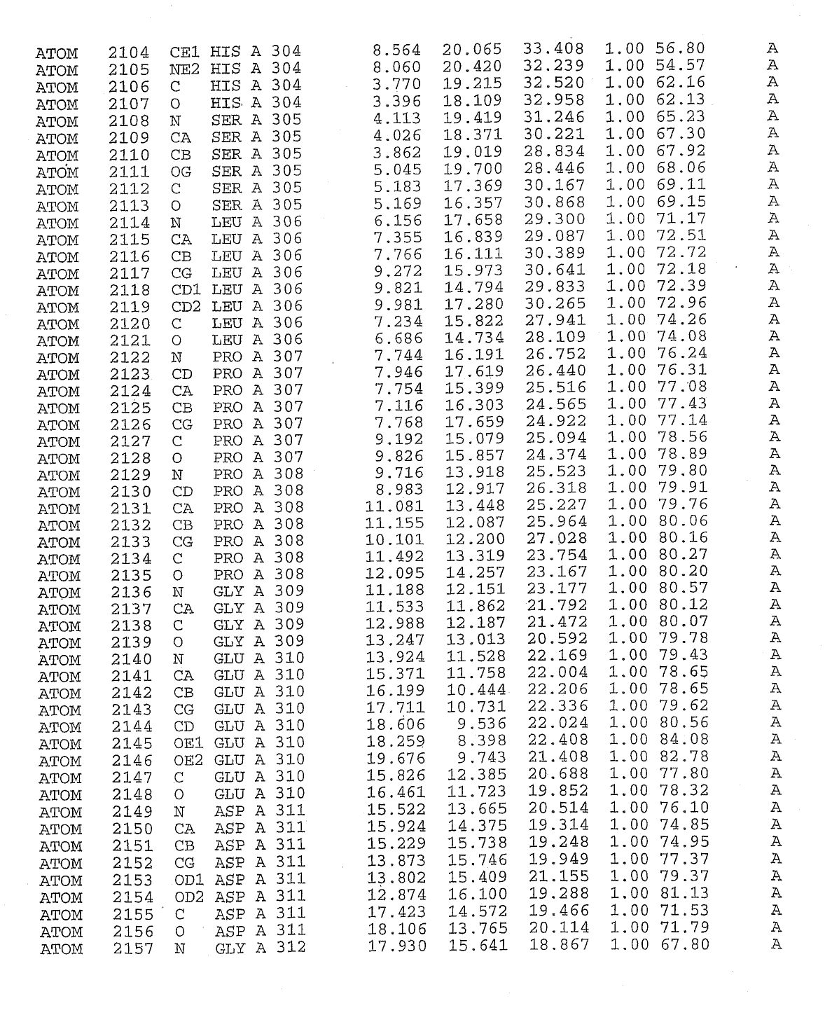

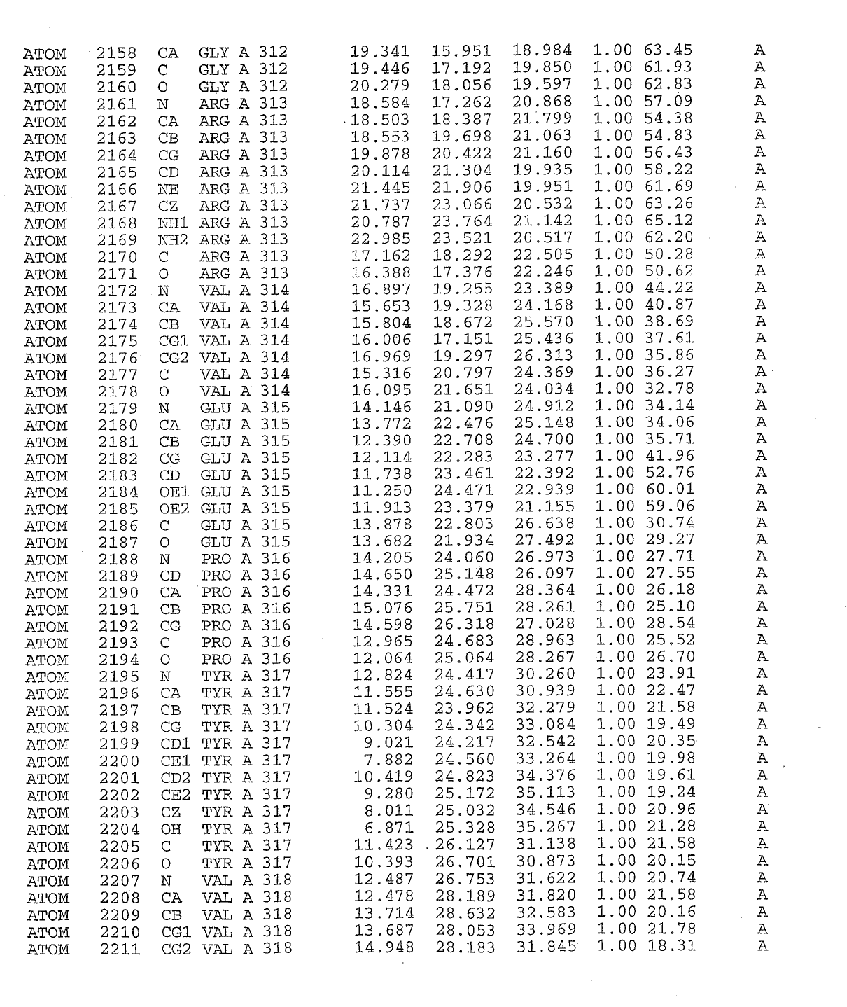

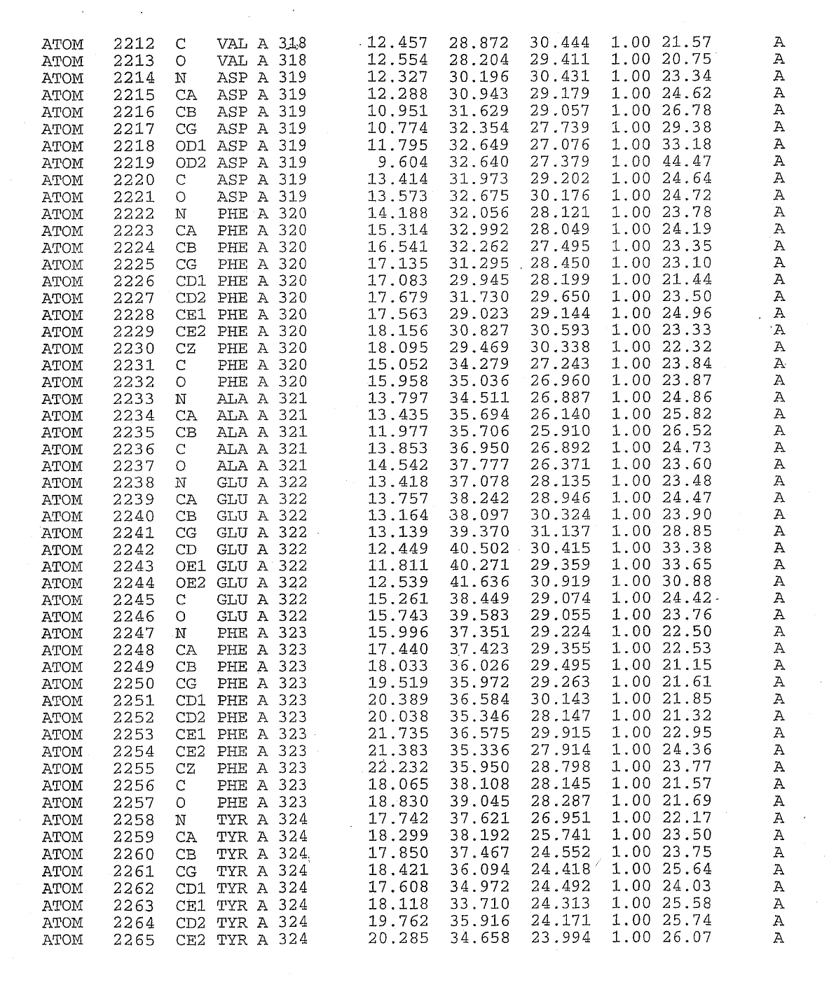

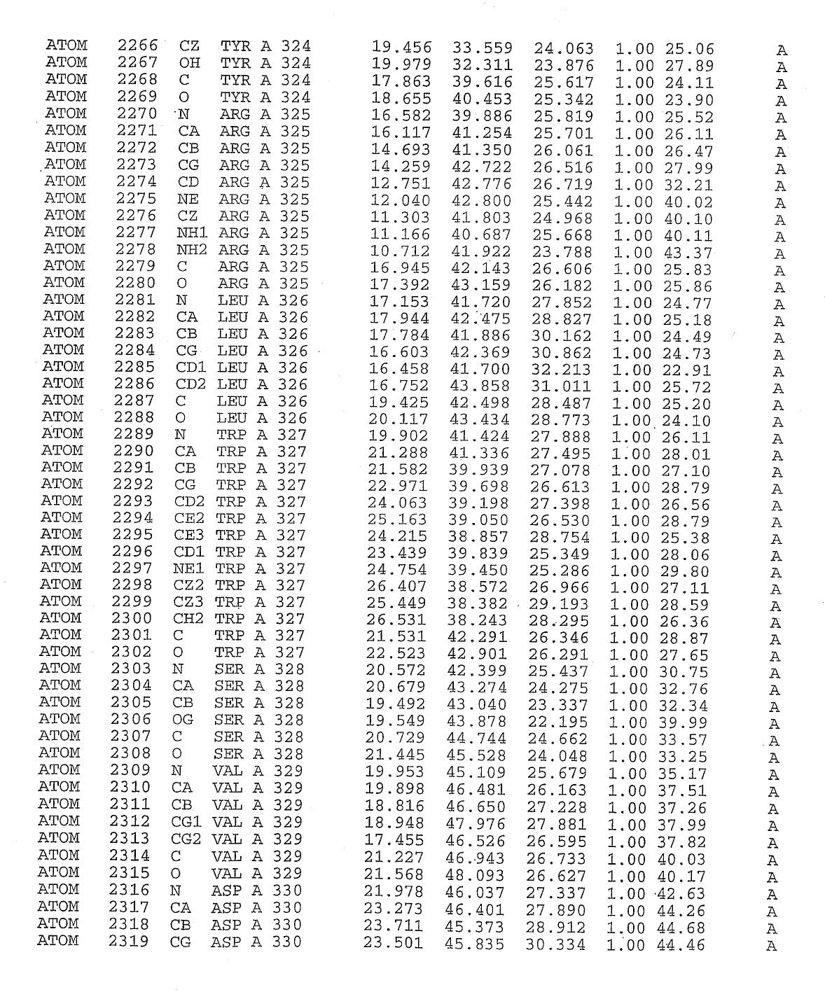

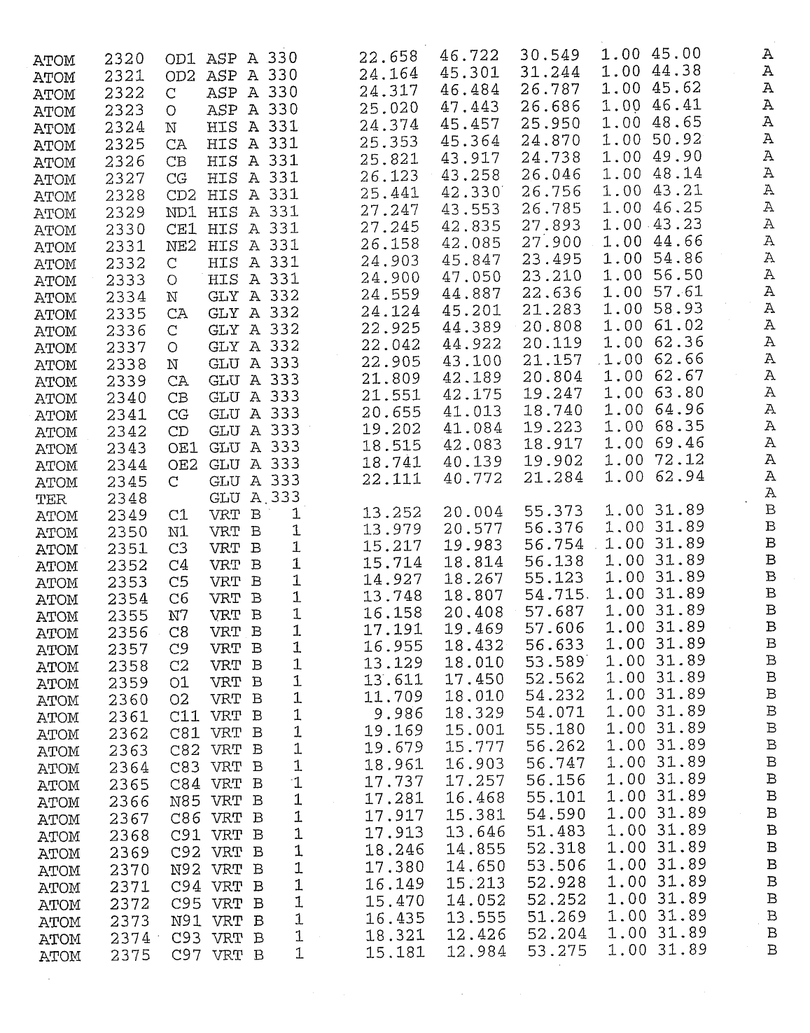

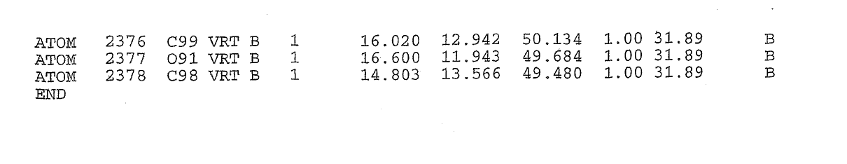

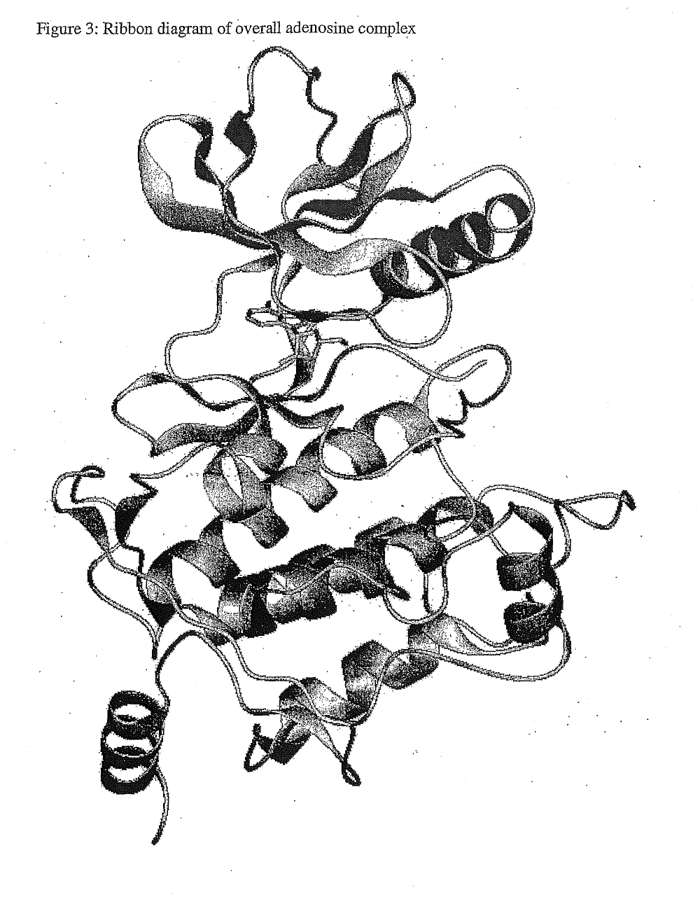

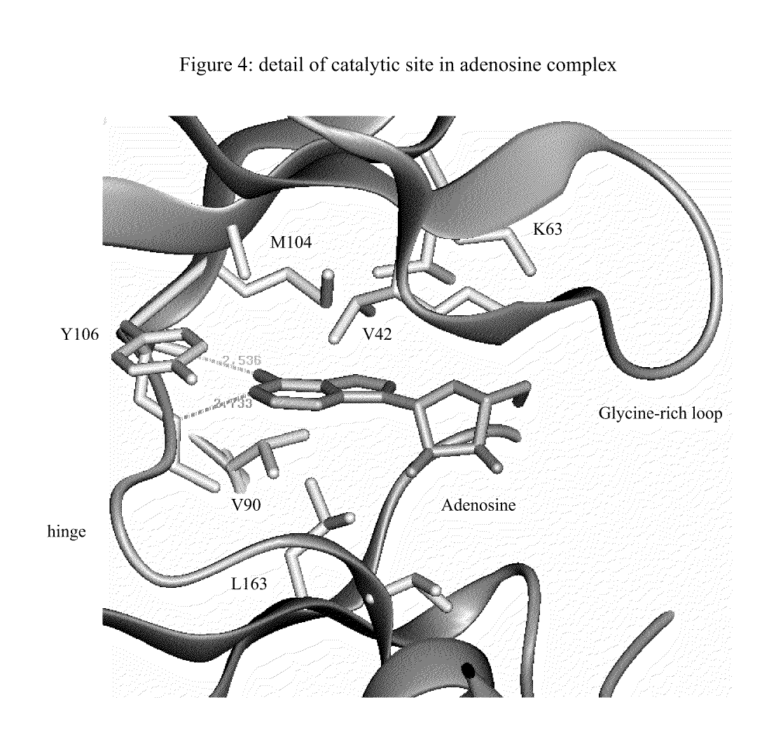

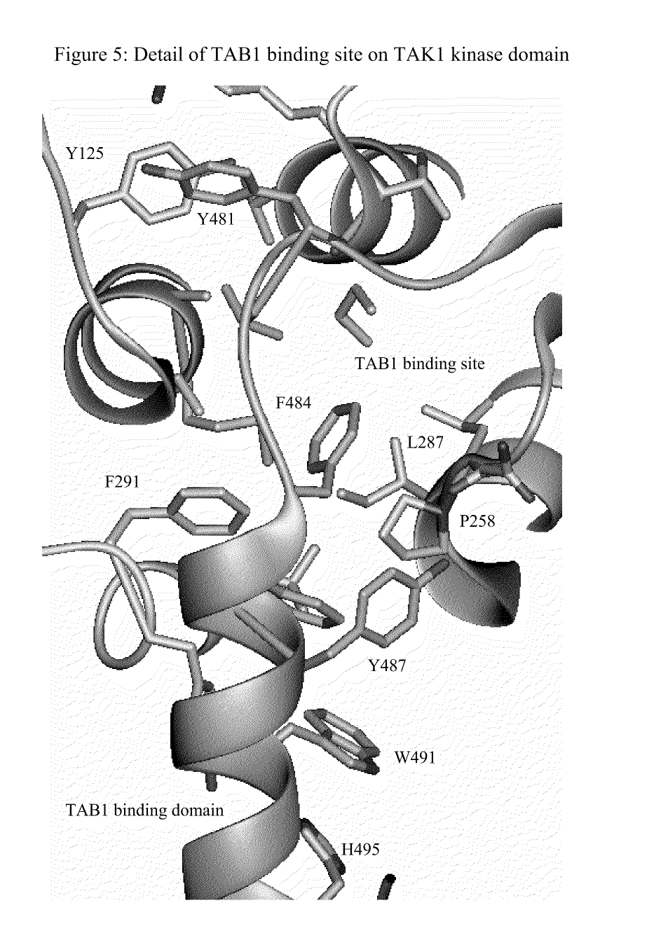

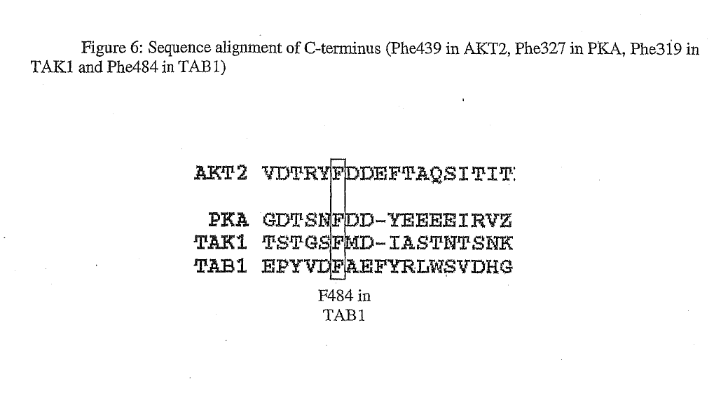

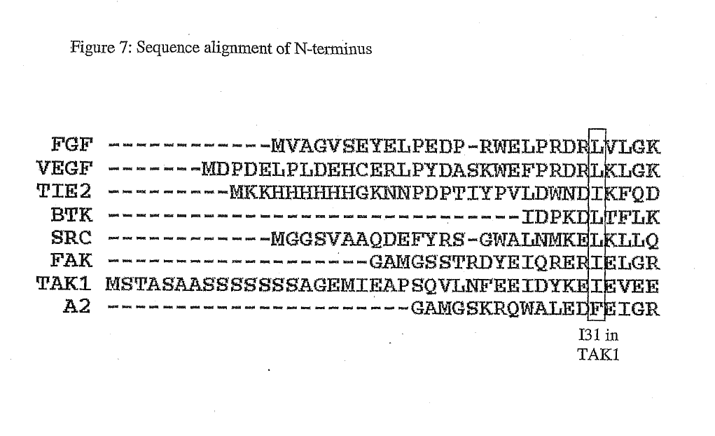

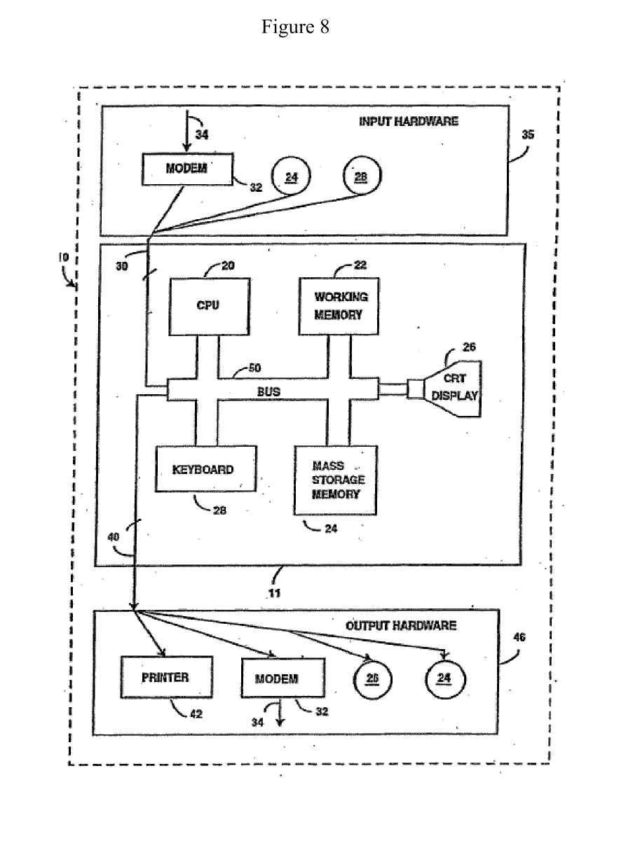

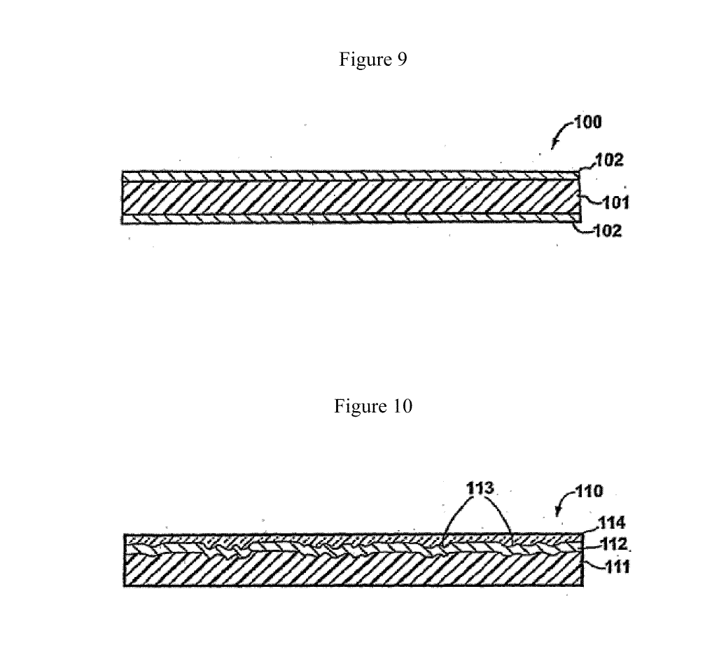

The following abbreviations are used in FIGS. 1-2:

“Atom type” refers to the element whose coordinates are measured. The first letter in the column defines the element.

“Resid” refers to the amino acid residue identity in the molecular model.

“X, Y, Z” crystallographically define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules of the crystal.

“Mol” refers to the molecule in the asymmetric unit.

FIG. 2 lists the atomic structure coordinates for the unphosphorylated TAK1 3-[6-(4-Acetyl-3,5-dimethyl-piperazin-1-yl)-pyridin-2-yl]-1H-pyrrolo[2,3 -b]pyridine-5-carboxylic acid methyl ester inhibitor complex as derived by X-ray diffraction from the crystal (Ser 27-Thr 178 of FIG. 2: SEQ ID NO: 17; Gly 191-Glu 333 of FIG. 2: SEQ ID NO: 18).

FIG. 3 depicts ribbon diagrams of the overall fold of TAK1—adenosine and TAK1-3-[6-(4-Acetyl-3,5-dimethyl-piperazin-1-yl)-pyridin-2-yl]-1H-pyrrolo[2,3-b]pyridine-5 -carboxylic acid methyl ester complexes. The N-terminal lobe of the TAK1 catalytic domain corresponds to the β-strand sub-domain and encompasses residues 31 to 104. The α-helical sub-domain corresponds to residues 112 to 303. Residues 304 to 340 correspond to the 36 C-terminal residues of TAB1. Key features of the kinase-fold such as the hinge (approximately residues 105 to 111), glycine rich loop (approximately residues 39 to 52) and activation loop or phosphorylation lip (approximately residues 175 to 191) are indicated. A number of residues in the activation loop (-178 to 520) and at the C-terminus (334 to 340) are disordered in each of the TAK1 crystal structures. They exhibited only weak electron density and could not be fitted.

FIG. 4 shows a detailed representation of pockets in the catalytic active site of the TAK1—adenosine complex.

FIG. 5 shows a detailed representation of pockets in the TAB1 binding site of the TAK1—TAB1 structure.

FIG. 6 shows a sequence alignment of the C-terminii of TAK1, TAB1 and AKT2, showing the conserved phenylalanine (Phe439 in AKT2 (SEQ ID NO: 1), Phe 327 in PKA (SEQ ID NO: 2), Phe319 in TAK1 (SEQ ID NO: 3), Phe484 in TAB1 (SEQ ID NO: 4)).

FIG. 7 shows a sequence alignment of the N-terminus of TAK1 with related protein kinases with close homology to TAK1 that have previously been crystallized in the literature (FGF: SEQ ID NO: 5; VEGF: SEQ ID NO: 6; TIE2: SEQ ID NO: 7; BTK: SEQ ID NO: 8; SRC: SEQ ID NO: 9; FAK: SEQ ID NO: 10; TAK1: SEQ ID NO: 11; A2: SEQ ID NO: 12).

FIG. 8 shows a diagram of a system used to carry out the instructions encoded by the storage medium of FIGS. 6 and 7.

FIG. 9 shows a cross section of a magnetic storage medium.

FIG. 10 shows a cross section of an optically-readable data storage medium.

### DETAILED DESCRIPTION OF THE INVENTION

In order that the invention described herein may be more fully understood, the following detailed description is set forth.

Throughout the specification, the word “comprise”, or variations such as “comprises” or “comprising” will be understood to imply the inclusion of a stated integer or groups of integers but not exclusion of any other integer or groups of integers.

The following abbreviations are used throughout the application:

| | | | | | |
|---|---|---|---|---|---|
| A = | Ala = | Alanine | T = | Thr = | Threonine |
| V = | Val = | Valine | C = | Cys = | Cysteine |
| L = | Leu = | Leucine | Y = | Tyr = | Tyrosine |
| I = | Ile = | Isoleucine | N = | Asn = | Asparagine |
| P = | Pro = | Proline | Q = | Gln = | Glutamine |
| F = | Phe = | Phenylalanine | D = | Asp = | Aspartic Acid |
| W = | Trp = | Tryptophan | E = | Glu = | Glutamic Acid |
| M = | Met = | Methionine | K = | Lys = | Lysine |
| G = | Gly = | Glycine | R = | Arg = | Arginine |
| S = | Ser = | Serine | H = | His = | Histidine |

Additional definitions are set forth herein.

The term “associating with” refers to a condition of proximity between a chemical entity or compound, or portions thereof, and a binding pocket or binding site on a protein. The association may be non-covalent—wherein the juxtaposition is energetically favored by hydrogen bonding or van der Waals or electrostatic interactions—or it may be covalent.

The term “binding pocket”, as used herein, refers to a region of a molecule or molecular complex, that, as a result of its shape and charge, favorably associates with another chemical entity or compound. The term “pocket” includes, but is not limited to, cleft, channel or site. TAK1 or TAK1—like molecules may have binding pockets which include, but are not limited to, peptide or substrate binding, ATP-binding and antibody binding sites.

The term “chemical entity”, as used herein, refers to chemical compounds, complexes of at least two chemical compounds, and fragments of such compounds or complexes. The chemical entity may be, for example, a ligand, a substrate, a nucleotide triphosphate, a nucleotide diphosphate, phosphate, a nucleotide, an agonist, antagonist, inhibitor, antibody, drug, peptide, protein or compound.

“Conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5, pp. 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

The term “corresponding amino acid” or “residue which corresponds to” refers to a particular amino acid or analogue thereof in a TAK1 homologue that corresponds to an amino acid in the TAK structure. The corresponding amino acid may be an identical, mutated, chemically modified, conserved, conservatively substituted, functionally equivalent or homologous amino acid when compared to the TAK1 amino acid to which it corresponds.

Methods for identifying a corresponding amino acid are known in the art and are based upon sequence, structural alignment, its functional position or a combination thereof as compared to the TAK1 structure. For example, corresponding amino acids may be identified by superimposing the backbone atoms of the amino acids in TAK1 and the TAK1 homologue using well known software applications, such as QUANTA [Molecular Simulations, Inc., San Diego, Calif. ©1998,2000]. The corresponding amino acids may also be identified using sequence alignment programs such as the “bestfit” program available from the Genetics Computer Group which uses the local homology algorithm described by Smith and Waterman in Adv. Appl. Math., 2, 482 (1981), which is incorporated herein by reference.

The term “domain” refers to a portion of the TAK1 protein or homologue that can be separated according to its biological function, for example, catalysis. The domain is usually conserved in sequence or structure when compared to other kinases or related proteins. The domain can comprise a binding pocket, or a sequence or structural motif.

The term “sub-domain” refers to a portion of the domain as defined above in the TAK1 protein or homologue. The catalytic kinase domain (amino acid residues 31 to 303) of TAK1 is a bi-lobal structure consisiting of an N-terminal, β-strand sub-domain (residues 31 to 104) and a C-terminal, α-helical sub-domain (residues 112 to 303).

The term “catalytic active site” refers to the area of the protein kinase to which nucleotide substrates bind. The catalytic active site of TAK1 is at the interface between the N-terminal, β-strand sub-domain and the C-terminal, α-helical sub-domain.

The “TAK1 ATP-binding pocket” of a molecule or molecular complex is defined by the structure coordinates of a certain set of amino acid residues present in the TAK1 structure, as described below. In general, the ligand for the ATP-binding pocket is a nucleotide such as ATP. This binding pocket is in the catalytic active site of the kinase domain. In the protein kinase family, the ATP-binding pocket is generally located at the interface of the α-helical and β-strand sub-domains, and is bordered by the glycine rich loop and the hinge [See, Xie et al., Structure, 6, pp. 983-991 (1998), incorporated herein by reference].

The “TAB1 binding pocket” of a molecule or molecular complex is defined by the structure coordinates of a certain set of amino acid residues present in the TAK1 structure, as described below. In general, the ligand for the TAB 1 binding pocket is the TAK1 binding domain of TAB1. This pocket is the site of association of TAK1 with its protein activator TAB1. The TAB1 binding pocket is located at near the bottom of the α-helical sub-domain, and is over 16 A long, 8 A wide and approximately 8 A deep, and is formed by residues belonging to the top of helices E and H and the tail of F and I.

The term “TAK1-like” refers to all or a portion of a molecule or molecular complex that has a commonality of shape to all or a portion of the TAK1 protein. In the TAK1-like ATP-binding pocket, the commonality of shape is defined by a root mean square deviation of the structure coordinates of the backbone atoms between the amino acids in the TAK1-like ATP-binding pocket and the amino acids in the TAK1 ATP-binding pocket (as set forth in FIG. 1, 2 or 3). Compared to an amino acid in the TAK1 ATP-binding pocket, the corresponding amino acids in the TAK1-like ATP-binding pocket may or may not be identical.

The term “part of an TAK1 ATP-binding pocket” or “part of an TAK1-like ATP-binding pocket” refers to less than all of the amino acid residues that define the TAK1 or TAK1-like ATP-binding pocket. The structure coordinates of residues that constitute part of an TAK1 or TAK1-like ATP-binding pocket may be specific for defining the chemical environment of the binding pocket, or useful in designing fragments of an inhibitor that may interact with those residues. For example, the portion of residues may be key residues that play a role in ligand binding, or may be residues that are spatially related and define a three-dimensional compartment of the binding pocket. The residues may be contiguous or non-contiguous in primary sequence. In one embodiment, part of the TAK1 or TAK1-like ATP-binding pocket is at least two amino acid residues, preferably, E105 and A107. In another embodiment, the amino acids are selected from the group consisting of V42, V90, M104, E105, A107 and L163.

The term “TAK1 kinase domain” refers to the catalytic domain of TAK1. The kinase domain includes, for example, the catalytic active site which comprises the catalytic residues, the activation loop or phosphorylation lip, the DFG motif, and the glycine-rich phosphate anchor or glycine-rich loop [See, Xie et al., Structure, 6, pp. 983-991 (1998); R. Giet and C. Prigent, J. Cell Sci., 112, pp. 3591-3601 (1999), incorporated herein by reference]. The kinase domain in the TAK1 protein comprises residues from about 31 to 303.

The term “part of a TAK1 kinase domain” or “part of a TAK1-like kinase domain” refers to a portion of the TAK1 or TAK1-like catalytic domain. The structure coordinates of residues that constitute part of a TAK1 or TAK1-like kinase domain may be specific for defining the chemical environment of the domain, or useful in designing fragments of an inhibitor that may interact with those residues. For example, the portion of residues may be key residues that play a role in ligand binding, or may be residues that are spatially related and define a three-dimensional compartment of the domain. The residues may be contiguous or non-contiguous in primary sequence. For example, part of a TAK1 kinase domain can be the active site, the glycine-rich loop, the activation loop, or the catalytic loop [see Xie et al., supra].

The term “homologue of TAK1” refers to a molecule or molecular complex that is homologous to TAK1 by three-dimensional structure or sequence. Examples of homologues include but are not limited to the following: human TAK1 with mutations, conservative substitutions, additions, deletions or a combination thereof; TAK1 from a species other than human; a protein comprising an TAK1-like ATP-binding pocket, a kinase domain; another member of the protein kinase family, preferably the MAPKKK kinase family or the CDK kinase family; or another member of the MAPK family of protein kinases. It should be understood that a homologue of TAK1 would include a truncation or extension of TAK1. Additionally, heavy atom derivatives of TAK1 may be obtained, for example, by soaking crystals in solutions containing a heavy atom compound. As is recognized, certain amino acids are particularly useful for preparing heavy atom derivatives, such as methionine, selenomethionine, cysteine, and selenocysteine. mutations are particularly useful for making heavy-atom derivative crystals. Such TAK1 homologues are within this invention.

Any of these changes are not mutually exclusive. A TAK1 homologue may have one or more than one of these changes. However, it is generally understood that in a homologue no more than about 20-30% of the amino acids should be changed relative to the wild-type protein. More specific homologues would be those wherein no more than about 25%, about 10%, or about 5% of the amino acids have been changed relative to the wild-type protein. In the case of protein crystals, a homologue should have no more than about 5-10% of the amino acids changed relative to the wild-type protein. More specific homologues would be those wherein no more than about 10%, about 5%, or about 1% of the amino acids have been changed relative to the wild-type protein. The wild-type TAK1 protein used herein is human TAK1. The wild-type TAB1 protein used herein is human TAK1.

TAB 1 homologues are similarly included in this invention.

The term “part of a TAK1 protein” or “part of a TAK1 homologue” refers to a portion of the amino acid residues of a TAK1 protein or homologue. In one embodiment, part of a TAK1 protein or homologue defines the binding pockets, domains, sub-domains, and motifs of the protein or homologue. The structure coordinates of residues that constitute part of a TAK1 protein or homologue may be specific for defining the chemical environment of the protein, or useful in designing fragments of an inhibitor that may interact with those residues. The portion of residues may also be residues that are spatially related and define a three-dimensional compartment of a binding pocket, motif or domain. The residues may be contiguous or non-contiguous in primary sequence. For example, the portion of residues may be key residues that play a role in ligand or substrate binding, peptide binding, antibody binding, catalysis, structural stabilization or degradation.

The term “TAK1 protein complex” or “TAK1 homologue complex” refers to a molecular complex formed by associating the TAK1 protein or TAK1 homologue with a chemical entity, for example, a ligand, a substrate, nucleotide triphosphate, an agonist or antagonist, inhibitor, drug or compound. In one embodiment, the chemical entity is selected from the group consisting of an ATP, a nucleotide triphosphate and an inhibitor for the ATP-binding pocket. In another embodiment, the inhibitor is an ATP analog such as MgAMP-PNP (adenylyl imidodiphosphate), adenosine, staurosporine or 3-(8-Phenyl-5,6-dihydrothieno[2,3-h]quinazolin-2-ylamino)benzenesulfonamide.

The term “motif” refers to a portion of the TAK1 protein or homologue that defines a structural compartment or carries out a function in the protein, for example, catalysis, structural stabilization, or phosphorylation. The motif may be conserved in sequence, structure and function when compared to other kinases or related proteins. The motif can be contiguous in primary sequence or three-dimensional space. The motif can comprise a-helices and/or (3-sheets. Examples of a motif include but are not limited to a binding pocket, active site, phosphorylation lip or activation loop, the glycine-rich phosphate anchor loop, the catalytic loop [See, Xie et al., Structure, 6, pp. 983-991 (1998); R. Giet and C. Prigent, J. Cell Sci., 112, pp. 3591-3601 (1999)], and the degradation box.

The term “root mean square deviation” or “RMSD” means the square root of the arithmetic mean of the squares of the deviations from the mean. It is a way to express the deviation or variation from a trend or object. For purposes of this invention, the “root mean square deviation” defines the variation in the backbone of a protein from the backbone of TAK1, a binding pocket, a motif, a domain, or portion thereof, as defined by the structure coordinates of TAK1 described herein.

The term “sufficiently homologous to TAK1” refers to a protein that has a sequence homology of at least 35% compared to TAK1 protein. In one embodiment, the sequence homology is at least 40%, at least 60%, at least 80%, at least 90% or at least 95%.

The term “soaked” refers to a process in which the crystal is transferred to a solution containing the compound of interest. In certain embodiments, the compound is diffused into the crystal.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex. It would be readily apparent to those skilled in the art that all or part of the structure coordinates of FIG. 1 (either molecule A or B) may have a RMSD deviation of 0.1 Å because of standard error.

The term “about” when used in the context of RMSD values takes into consideration the standard error of the RMSD value, which is ±0.1Å.

The term “crystallization solution” refers to a solution that promotes crystallization. The solution comprises at least one agent, and may include a buffer, one or more salts, a precipitating agent, one or more detergents, sugars or organic compounds, lanthanide ions, a poly-ionic compound and/or a stabilizer.

The term “generating a three-dimensional structure” or “generating a three-dimensional graphical representation” refers to converting the lists of structure coordinates into structural models in three-dimensional space. This can be achieved through commercially or publicly available software. The three-dimensional structure may be displayed as a graphical representation or used to perform computer modeling or fitting operations. In addition, the structure coordinates themselves may be used to perform computer modeling and fitting operations.

The term “homologue of TAK1” or “TAK1 homologue” refers to a molecule that is homologous to TAK1 by three-dimensional structure or sequence and retains the kinase activity of TAK1. Examples of homologues include, but are not limited to, TAK1 having one or more amino acid residues that are chemically modified, mutated, conservatively substituted, added, deleted or a combination thereof.

The term “homology model” refers to a structural model derived from known three-dimensional structure(s). Generation of the homology model, termed “homology modeling”, can include sequence alignment, residue replacement, residue conformation adjustment through energy minimization, or a combination thereof

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for a TAK1 molecule or molecular complex or homologues thereof, calculating or minimizing energies for an association of a TAK1 molecule or molecular complex or homologues thereof to a chemical entity.

Rational Design of TAK1 Proteins for Crystallization

According to another embodiment, the invention provides a method for designing crystallizable TAK-1 proteins. Initial attempts at crystallization of a TAK1-TAB1 complex based on a previously reported fusion protein (Sakurai H., Nishi A., Sato N., Mizukami J., Miyoshi H. and Sugita T. Biochem Biophys Res Comm. 297 1277-1281 (2002)) proved unsuccessful and so a shorter TAK1 construct comprising 11e31-Gln303 fused directly to the C-terminal 36 residues of TAB1 (His468-Pro504) was expressed in baculovirus and purified. The choice of C-terminus was based on the sequence alignment of the TAK1 (Tyr304-Pro339), TAB1 (His468-Pro504) and AKT2 (Leu423-Leu459) (FIG. 6). AKT2 is a member of the AGC kinase family and requires the protein activator PDK1 to induce full activation (Frodin M, Antal T L, Dummler B A, Jensen C J, Deak M, Gammeltoft S, Biondi R M EMBO J. 2002 Oct 15;21(20):5396-407). The alignment highlights a conserved phenylalanine residue (Phe439 in AKT2, Phe319 in TAK1, Phe484 in TAB1), which in AKT2 lies adjacent to the ATP binding site (Yang J, Cron P, Thompson V, Good V M, Hess D, Hemmings B A, Barford D. Mol Cell. 2002 June;9(6):1227-40.). The TAK1-TAB1 chimera was designed by replacing the C-terminal residues of TAK1 with the corresponding residues of TAB1 preserving the conserved phenylalanine An additional 18 residues were selected N-terminal to the conserved phenylalanine, up to and including Tyr304 of TAK1, as this preserved a conserved serine residue (Ser305 in TAK1, Ser469 in TAB1). A total of 36 residues were thereby replaced and TAK1 and TAB 1 were fused together without a linker region to assist crystallization by removing structurally unimportant residues. Without being bound by theory it is believed that it is advantageous to eliminate the unwieldy linker that had been used in previously studied fusion protein (Glu-Phe-(Gly)5) (SEQ ID NO: 13)). The N-terminus of the TAK1 kinase domain was chosen based on both sequence and structural alignments of related protein kinases with close homology to TAK1 that have previously been crystallized in the literature. This revealed an interaction between residue 131, a highly conserved hydrophobic residue in the N-terminus of β-strand β1, and a neighbouring hydrophobic pocket.

Applicants have observed that both the hydrophobic nature of this residue and hydrophobic nature of the neighbouring pocket is retained in kinases giving crystallizable proteins (FIG. 7). Consequently, residue 131 was chosen as the N-terminus of the TAK1-TAB1 chimera. The choice of N- and C-termini was validated since the construct both retained catalytic activity and crystallized.

### Crystallizable Compositions and Crystals of TAK1 Complexes

This invention provides an isolated, purified TAK1 protein and a crystal thereof

According to one embodiment, the invention provides a crystallizable composition comprising unphosphorylated TAK1 protein. In another embodiment, the invention provides a crystallizable composition comprising unphosphorylated TAK1 protein and a substrate analogue, such as but not limited to adenosine. In one embodiment, the aforementioned crystallizable composition further comprises a precipitant, 600-900 mM sodium citrate (the precipitant), 1 to 200 mM sodium chloride and a buffer that maintains pH at between about 6.5 and about 8.5. The composition may further comprise a reducing agent, such as dithiothreitol (DTT) at between about 1 to about 20 mM. The unphosphorylated TAK1 protein or complex is preferably about 85-100% pure prior to forming the composition.

According to another embodiment, the invention provides a crystal composition comprising TAK1 protein complex. In one embodiment, the crystal has a unit cell dimension of a=58Å, b=144Å, c=134Å, α=β=γ=90° and belongs to space group I222. It will be readily apparent to those skilled in the art that the unit cells of the crystal compositions may deviate ±1-2Å from the above cell dimensions depending on the deviation in the unit cell calculations.

As used herein, the TAK1 protein in the crystal or crystallizable compositions can be a truncated protein with amino acids 31 to 340 as shown in FIGS. 1-3; and the truncated protein with conservative substitutions.

The TAK1 protein may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products. Preferably, the protein is overexpressed from a baculovirus system.

The invention also relates to a method of making crystals of TAK1 complexes or TAK1 homologue complexes. Such methods comprise the steps of:

a) producing a composition comprising a crystallization solution and TAK1 protein or homologue thereof complexed with a chemical entity; and

b) subjecting said composition to devices or conditions which promote crystallization.

In one embodiment, the chemical entity is selected from the group consisting of an ATP analogue, nucleotide triphosphate, nucleotide diphosphate, phosphate, adenosine, or active site inhibitor. In another embodiment, the chemical entity is an ATP analogue. In certain exemplary embodiments, the chemical entity is 3-(8-Phenyl-5,6-dihydrothieno[2,3-h]quinazolin-2-ylamino)benzenesulfon-amide. In yet another embodiment, the crystallization solution is as described previously. In another embodiment, the composition is treated with micro-crystals of TAK1 or TAK1 complexes or homologues thereof. In another embodiment, the composition is treated with micro-crystals of TAK1 complexes or homologues thereof.

In certain embodiments, the invention provides a method of making TAK1 crystals, the method comprising steps of:

a) producing and purifying TAK1 protein;
b) producing a crystallizable composition; and
c) subjecting said composition to devices which promote crystallization.

In one embodiment, the crystallizable composition of step b) is made according to the conditions dislcosed above.

Devices for promoting crystallization can include but are not limited to the hanging-drop, sitting-drop, dialysis or microtube batch devices. [U.S. Pat. No. 4,886,646, 5,096,676, 5,130,105, 5,221,410 and 5,400,741; Pay et al., Proteins: Structure, Function, and Genetics, 20, pp. 98-102 (1994), incorporated herein by reference]. The hanging-drop or sitting-drop methods produce crystals by vapor diffusion. The hanging-drop, sitting-drop, and some adaptations of the microbatch methods [D'Arcy et al., J. Cryst. Growth, 168, pp. 175-180 (1996) and Chayen, J. Appl. Cryst., 30, pp. 198-202 (1997)] produce crystals by vapor diffusion. The hanging drop and sitting drop containing the crystallizable composition is equilibrated in a reservoir containing a higher or lower concentration of the precipitant. As the drop approaches equilibrium with the reservoir, the saturation of protein in the solution leads to the formation of crystals.

Microseeding or seeding may be used to obtain larger, or better quality (i.e., crystals with higher resolution diffraction or single crystals) crystals from initial micro-crystals. Microseeding involves the use of crystalline particles to provide nucleation under controlled crystallization conditions. Microseeding is used to increase the size and quality of crystals. In this instance, micro-crystals are crushed to yield a stock seed solution. The stock seed solution is diluted in series. Using a needle, glass rod or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art following the teachings of the specification to vary the crystallization conditions disclosed herein to identify other crystallization conditions that would produce crystals of TAK1 homologue, TAK1 homologue complex, TAK1 protein or other TAK1 protein complexes. Such variations include, but are not limited to, adjusting pH, protein concentration and/or crystallization temperature, changing the identity or concentration of salt and/or precipitant used, using a different method of crystallization, or introducing additives such as detergents (e.g., TWEEN 20 (monolaurate), LDAO, Brij 30 (4 lauryl ether)), sugars (e.g., glucose, maltose), organic compounds (e.g., dioxane, dimethylformamide), lanthanide ions or polyionic compounds that aid in crystallization. High throughput crystallization assays may also be used to assist in finding or optimizing the crystallization conditions.

### Binding Pockets of TAK1 Protein or Homologues Thereof

As disclosed above, applicants have provided for the first time the three-dimensional X-ray crystal structures of a TAK1 - inhibitor complex. The crystal structure of TAK1 presented here is the first reported for TAK1. The invention will be useful for inhibitor design to study the role of TAK1 in cell signaling. The atomic coordinate data is presented in FIGS. 1-3.

In order to use the structure coordinates generated for TAK1, their complexes, one of their binding pockets, or a TAK1-like binding pocket thereof, it is often at times necessary to convert the coordinates into a three-dimensional shape. This is achieved through the use of commercially available software that is capable of generating three-dimensional graphical representations (e.g., three-dimensional structures) of molecules or portions thereof from a set of structure coordinates.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential inhibitors of the binding pockets of biologically important targets. The ATP and substrate binding pockets of this invention will be useful for drug design.

Applicants invention has confirmed that amino acids E105 and A107 are in the hinge region of the TAK1 protein. A binding pocket comprising these amino acids would be useful for drug design. Accordingly, in one embodiment, this invention provides a binding pocket comprising amino acids E105 and A107.

In another embodiment, the ATP-binding pocket comprises amino acids V42, G43, V50, A61, K63, V90, M104, E105, Y106, A107, E108, G109, 5111, N114, L163 and C174 using the structure of the TAK1 - adenosine complex according to FIG. 1. In another embodiment, the ATP-binding pocket comprises amino acids V42, G43, V50, A61, K63, V90, M104, E105, Y106, A107, E108, G109, 5111, N114, L163 and C174 using the structure of the TAK1-3-[6-(4-Acetyl-3,5-dimethyl-piperazin-1-yl)-pyridin-2-yl] -1H-pyrrolo[2,3-b]pyridine-5-carboxylic acid methyl ester complex according to FIG. 2.

In resolving the crystal structures of the unphosphorylated TAK1—inhibitor complexes, applicants have determined that the above amino acids are within 5Å (“5Å sphere amino acids”) of the inhibitor bound in the ATP-binding pockets. These residues were identified using the program QUANTA [Molecular Simulations, Inc., San Diego, Calif. ©1998,2000], O [T. A. Jones et al., Acta Cryst. A, 47, pp. 110-119 (1991)] and RIBBONS [Carson, J. Appl. Cryst., 24, pp. 958-961 (1991)]. The programs allow one to display and output all residues within 5Å from the inhibitor. Thus, a binding pocket defined by the structural coordinates of these amino acids, as set forth in FIGS. 1 and 2 is considered an TAK1-ATP binding pocket of this invention.

In another embodiment, the ATP-binding pocket comprises amino acids E40, V42, G43, G48, V50, C51, K52, D59, V60, A61, 162, K63, V90, L97, L102, V103, M104, E105, Y106, A107, E108, G109, S111, L112, Y113, N114, P160, N161, L162, L163, L164, K172, 1173, C174 and D175 using the structure of the TAK1- adenosine complex to FIG. 1. In another embodiment, the ATP-binding pocket comprises amino acids E40, V42, G43, G48, V50, C51, K52, D59, V60, A61, 162, K63, V90, L97, L102, V103, M104, E105, Y106, A107, E108, G109, S111, L112, Y113, N114, P160, N161, L162, L163, L164, K172, 1173, C174 and D175 using the structure of the TAK1—3-[6-(4-Acetyl-3,5 -dimethyl-piperazin-1-yl)-pyridin-2-yl]-1H-pyrrolo[2,3-b]pyridine-5-carboxylic acid methyl ester complex_according to FIG. 2. In the crystal structures of the TAK1—inhibitor complexes, applicants have determined that the above amino acids are within 8Å (“8Å sphere amino acids”) of the inhibitor bound in the ATP-binding pockets. These residues were identified using the programs QUANTA, O and RIBBONS, supra. Thus, a binding pocket defined by the structural coordinates of these amino acids, as set forth in FIGS. 1 and 2 is considered an TAK1-ATP binding pocket of this invention.

In another embodiment, the ATP-binding pocket comprises amino acids 136, V38, E39, E40, A46, G48, V50, C51, K52, A53, K54, WW55, D59, V60, A61, 162, K63, Q64, L78, R79, Q80, H86, N88, 189, V90, L97, L92, Y93, G94, A95, C96, V100, C101, L102, V103, M104, E105, Y106, A107, E108, G109, S111, L112, Y113, N114, V115, H129, A130, M131, 5132, W133, C134, L135, Q136, C137, 5138, Q139, G140, V141, A142, Y143, L144, H145, 5146, M147, A151, L152, 1153, H154, R155, D156, L157, K158, P159, P160, N161, L162, L163, L164, V165, A166, T169, V170, L171, K172, 1173, C174 and D175 using the structure of the TAK1—adenosine complex to FIG. 1. In another embodiment, the ATP-binding pocket comprises amino acids 136, V38, E39, E40, A46, G48, V50, C51, K52, A53, K54, WW55, D59, V60, A61, 162, K63, Q64, L78, R79, Q80, H86, N88, 189, V90, L97, L92, Y93, G94, A95, C96, V100, C101, L102, V103, M104, E105, Y106, A107, E108, G109, S111, L112, Y113, N114, V115, H129, A130, M131, S132, W133, C134, L135, Q136, C137, S138, Q139, G140, V141, A142, Y143, L144, H145, S146, M147, A151, L152, L153, H154, R155, D156, L157, K158, P159, P160, N161, L162, L163, L164, V165, A166, T169, V170, L171, K172, L173, C174 and D175 using the structure of the TAK1—3-[6-(4-Acetyl-3,5-dimethyl-piperazin-1-yl)-pyridin-2-yl]-1H-pyrrolo[2,3-b]pyridine-5-carboxylic acid methyl ester complex according to FIG. 2. Using a multiple alignment program to compare each TAK1 structure and structures of other members of the protein kinase family [Gerstein et al., J. Mol. Biol., 251, pp. 161-175 (1995), incorporated herein by reference], applicants have identified the above amino acids as the ATP-binding pocket. First, a sequence alignment between members of the protein kinase family including Aurora-2 [PDB Accession number 1MUO], p38 [K. P. Wilson et al., J. Biol. Chem., 271, pp. 27696-27700 (1996); Z. Wang et al., Proc. Natl. Acad. Sci. U.S.A., 94, pp. 2327-32 (1997)], CDK2 [PDB Accession number 1B38], SRC [Xu, W., et al., Cell 3, pp. 629-638 (1999); PDB Accession number 2SRC], MK2 [U.S. Provisional application 60/337,513] and LCK [Yamaguchi H., Hendrickson W. A., Nature. 384, pp. 484-489 (1996); PDB Accession number 3LCK] is performed. Then, a putative core is constructed by superimposing a series of corresponding structures in the protein kinase family. Then, residues of high spatial variation are discarded, and the core alignment is iteratively refined. The amino acids that make up the final core structure have low structural variance and have the same local and global conformation relative to the corresponding residues in the protein family.

In one embodiment, the ATP-binding pocket comprises the amino acids of V42, V90, M104, E105, A107 and L163 according to FIGS. 1 and 2. It will be readily apparent to those of skill in the art that the numbering of amino acids in other homologues of TAK1 may be different than that set forth for TAK1. Corresponding amino acids in homologues of TAK1 are easily identified by visual inspection of the amino acid sequences or by using commercially available sequence homology, structural homology or structure superimposition software programs.

This invention also provides a novel TAB1 binding pocket on the kinase domain. This binding pocket is located on the bottom of the C-lobe and not near the ATP binding pocket. This TAB1-binding pocket will be useful for drug design.

Accordingly, another embodiment of this invention provides a TAK1 TAB1-binding pocket defined by structure coordinates of TAK1 amino acids M131, P256, Y290 and F291 according to FIG. 1; or a TAK1-like TAB1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a TAK1-like TAB 1-binding pocket defined by structure coordinates of a set of corresponding amino acids, wherein the root mean square deviation of the backbone atoms between said set of corresponding amino acids and said TAK1 amino acids is not more than about 1.1Å, 0.9Å, 0.7Å, or 0.5Å and wherein at least one of said corresponding amino acids is not identical to the TAK1 amino acid to which it corresponds.

Another embodiment of this invention provides a TAK1 TAB1-binding pocket defined by structure coordinates of TAK1 amino acids A127, M131, P256, 1259, Y290 and F291 according to FIG. 1; or a TAK1-like TAB1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a TAK1-like TAB 1-binding pocket defined by structure coordinates of a set of corresponding amino acids, wherein the root mean square deviation of the backbone atoms between said set of corresponding amino acids and said TAK1 amino acids is not more than about 1.0Å, 0.8Å, or 0.6Å, and wherein at least one of said corresponding amino acids is not identical to the TAK1 amino acid to which it corresponds.

Another embodiment of this invention provides a TAK1 TAB1-binding pocket defined by structure coordinates of TAK1 amino acids M131, P256, Y290 and F291 according to FIG. 2; or a TAK1-like TAB 1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a TAK1-like TAB 1-binding pocket defined by structure coordinates of a set of corresponding amino acids, wherein the root mean square deviation of the backbone atoms between said set of corresponding amino acids and said TAK1 amino acids is not more than about 1.1Å, 0.9Å, 0.7Å, or 0.5Å and wherein at least one of said corresponding amino acids is not identical to the TAK1 amino acid to which it corresponds.

Another embodiment of this invention provides a TAK1 TAB1-binding pocket defined by structure coordinates of TAK1 amino acids A127, M131, P256, 1259, Y290 and F291 according to FIG. 2; or a TAK1-like TAB 1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a TAK1-like TAB 1-binding pocket defined by structure coordinates of a set of corresponding amino acids, wherein the root mean square deviation of the backbone atoms between said set of corresponding amino acids and said TAK1 amino acids is not more than about 1.0Å, 0.8Å, or 0.6Å, and wherein at least one of said corresponding amino acids is not identical to the TAK1 amino acid to which it corresponds.

Applicants have also determined a unique re-orientation of the N-lobe relative to the C-lobe. This determination was crucial in solving the three-dimensional structure of TAK 1 .

Those of skill in the art understand that a set of structure coordinates for a molecule or a molecular-complex or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates indicated above may be generated because of mathematical manipulations of the TAK1 structure coordinates. For example, the structure coordinates set forth in FIGS. 1 and 2 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of TAK1 would also be expected to bind to another binding pocket whose structure coordinates defined a shape that fell within the acceptable root mean square deviation.

Various computational analyses maybe necessary to determine whether a binding pocket, motif, domain or portion thereof of a molecule or molecular complex is sufficiently similar to the binding pocket, motif, domain or portion thereof of TAK1. Such analyses may be carried out in well known software applications, such as ProFit [A. C. R. Martin, SciTech Software, ProFit version 1.8, University College London, http://www.bioinforg.uk/software], Swiss-Pdb Viewer [Guex et al., Electrophoresis, 18, pp. 2714-2723 (1997)], the Molecular Similarity application of QUANTA [Molecular Simulations Inc., San Diego, Calif. © 1998,2000] and as described in the accompanying User's Guide, which are incorporated herein by reference.

The above programs permit comparisons between different structures, different conformations of the same structure, and different parts of the same structure. The procedure used in QUANTA [Molecular Simulations, Inc., San Diego, CA ©1998,2000] and Swiss-Pdb Viewer to compare structures is divided into four steps: 1) load the structures to be compared; 2) define the atom equivalences in these structures; 3) perform a fitting operation on the structures; and 4) analyze the results. The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within the above programs is defined by user input, for the purpose of this invention we will define equivalent atoms as protein backbone atoms (N, Cα, C and O) for TAK1 amino acids and corresponding amino acids in the structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482 (1981), which is incorporated herein by reference. A suitable amino acid sequence alignment will require that the proteins being aligned share minimum percentage of identical amino acids. Generally, a first protein being aligned with a second protein should share in excess of about 35% identical amino acids with the second protein [Hanks et al., Science, 241, 42 (1988); Hanks and Quinn, Meth. Enzymol,, 200, 38 (1991)]. The identification of equivalent residues can also be assisted by secondary structure alignment, for example, aligning the a-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. For programs that calculate an average of the individual RMSD values of the backbone atoms, an RMSD cutoff value can be used to exclude pairs of equivalent atoms with extreme individual RMSD values. In the program ProFit, the RMSD cutoff value can be specified by the user.

The RMSD values between other protein kinases the TAK1 protein complexes (FIGS. 1 and 2) and other kinases are illustrated in Table 1. The RMSD values were determined by the programs ProFit from initial rigid fitting results from QUANTA. The RMSD values provided in Table 1 are averages of individual RMSD values calculated for the backbone atoms in the kinase or ATP-binding pocket. The RMSD cutoff value in ProFit was specified as 3Å.

For the 5Å and 8Å sphere amino acids, the values for the RMSD values of the ATP-binding pocket between the TAK1—3-[6-(4-Acetyl-3,5-dimethyl-piperazin-1-yl)-pyridin-2-yl]-1H-pyrrolo[2,3-b]pyridine-5-carboxylic acid methyl ester inhibitor complex and the TAK1—adenosine complex are 0.33Å and 0.30Å, respectively. The comparison of the whole kinase domain yields RMSD values of 0.11Å using the TAK1—3-[6-(4-Acetyl-3,5-dimethyl-piperazin-1-yl)-pyridsin-2-yl]-1H-pyrrolo[2,3-b]pyridine-5-carboxylic acid methyl ester inhibitor complex as a reference.

TABLE 1
RMSD values for TAK1-3-[6-(4-Acetyl-3,5-dimethyl-
piperazin-1-yl)-pyridin-2-yl]-1H-pyrrolo[2,3-b]pyridine-5-
carboxylic acid methyl ester inhibitor complex

| | RMSD value | RMSD value | RMSD value |
|---|---|---|---|
| | between ATP- | between ATP- | between TAK- |
| | binding pocket (8 Å | binding pocket (5 Å | complex |
| | sphere of amino | sphere of amino | kinase |
| | acids) and | acids) and | domain and |
| | corresponding | corresponding | kinase |
| | amino | amino | domain |
| | acids in | acids in | in protein |
| Protein | protein (Å) | protein (Å) | (Å) |
| Aur-2^{a} | 1.87 | 2.11 | 2.36 |
| P38^{b} | 3.63 | 1.50 | 8.56 |
| FLT-3^{c} | 2.11 | 0.92 | 2.53 |
| ITK^{d} | 1.54 | 0.69 | 2.55 |
| MK2^{e} | 1.15 | 1.25 | 8.84 |
| LCK^{f} | 2.07 | 1.27 | 1.86 |
| Adenosine^{g} | 0.30 | 0.33 | 0.11 |

^{a}Aurora-2 kinase: United States Provisional application VPI/01-12_which is U.S. Provisional Application No. 60/377,510, filed on May 1, 2002, expired.
^{b}p38: Wilson et al., J. Biol. Chem., 271, pp. 27696-27700 (1996); Z. Wang et al., Proc. Natl. Acad. Sci. U.S.A., 94, pp. 2327-2332 (1997); PDB Accession number 1WFC.
^{c}FMS-like Tyrosine Kinase 3 (FLT3): Griffith et al., et al., Mol. Cell. 13, pp. 169 (2004); PDB Accession number 1RJB.
^{d}Interleukin-2 Tyrosine Kinase: Brown et al., J. Biol. Chem. 279, pp. 18727 (2004); PDB Accession number 1SM2.
^{e}Mitogen activated protein kinase activated protein (MAPKAP) kinase 2: United States Provisional application 60/337,513.
^{f}Lymphocyte-specific kinase (LCK):ref Yamaguchi H., Hendrickson W.A., Nature. 384, pp. 484-489 (1996); PDB Accession number 3LCK.
^{g}TAK-1/TAB-1 Adenosine complex (this application).

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in FIGS. 1 and 2 are encompassed by this invention.

Therefore, one embodiment of this invention provides a molecule or molecular complex comprising all or part of a TAK1 ATP-binding pocket as defined herein. Another embodiment of this invention provides a molecule or molecular complex comprising all or part of a TAK1 TAB1-binding pocket defined.

In one embodiment, the above molecules or molecular complexes are in crystalline form.

### Computer Systems

As would be recognized, this invention is ideally suited for use in computer-implemented inventions. Accordingly, this invention provides a computer system comprising one or more of a) atomic coordinate data as disclosed herein +/− a root mean square deviation from the Ca atoms of not more than 1.5Å (or 1.0Å or 0.5Å); b) structure factor data (where a structure factor comprises the amplitude and phase of the diffracted wave) for TAK1, said structure factor data being derivable from the atomic coordinate data of Table 1 ±a root mean square deviation from the Ca atoms of not more than 1.5Å (or 1.0Å or 0.5Å); c) atomic coordinate data of a target TAK1 protein generated by homology modeling of the target based on the data disclosed herein ± a root mean square deviation from the Ca atoms of not more than 1.5Å (or 1.0Å or 0.5Å); d) atomic coordinate data of a target TAK1 protein generated by interpreting X-ray crystallographic data or NMR data by reference to the data disclosed herein ±a root mean square deviation from the Ca atoms of not more than 1.5Å (or 1.0Å or 0.5Å); or (e) structure factor data a derivable from the atomic coordinate data of (c) or (d). In certain embodiments, a computer system comprises: a computer-readable data storage medium comprising data storage material encoded with the computer -readable data; (a) a working memory for storing instructions for processing said computer -readable data; and (b) a central-processing unit coupled to said working memory and to said computer -readable data storage medium for processing said computer—readable data and thereby generating structures and/or performing rational drug design

According to another embodiment of this invention is provided a machine-readable data storage medium, comprising a data storage material encoded with machine-readable data, wherein said data comprises all or part of an TAK1 ATP-binding pocket defined by structure coordinates of TAK1 amino acids V42, G43, V50, A61, K63, V90, M104, E105, A107, E108, G109, S111, N114, L163 and C174, according to FIG. 1; or a molecule or molecular complex comprising all or part of an TAK1-like ATP-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a molecule or molecular complex comprising all or part of an TAK1-like ATP-binding pocket defined by structure coordinates of a set of corresponding amino acids, wherein the root mean square deviation of the backbone atoms between said set of corresponding amino acids and said TAK1 amino acids is not more than about 1.1, 0.9, 0.7 or 0.5Å, and wherein at least one of said corresponding amino acids is not identical to the TAK1 amino acid to which it corresponds.

In other embodiments of this invention is provided a machine-readable data storage medium, comprising a data storage material encoded with machine-readable data, wherein said data comprises all or part of any molecule or molecular complex disclosed herein. Alternatively, the data storage material is encoded with machine-readable data comprising all or part of a binding pocket of this invention or a TAK1 according to this invention.

In one embodiment of this invention is provided a computer comprising:

a machine-readable data storage medium, comprising a data storage material encoded with machine-readable data, wherein said data comprises all or part of a TAK1 ATP-binding pocket defined by structure coordinates of TAK1 amino acids V42, G43, V50, A61, K63, V90, M104, E105, A107, E108, G109, S111, N114, L163 and C174, according to FIG. 1; or a molecule or molecular complex comprising all or part of a TAK1-like ATP-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a molecule or molecular complex comprising all or part of a TAK1-like ATP-binding pocket defined by structure coordinates of a set of corresponding amino acids, wherein the root mean square deviation of the backbone atoms between said set of corresponding amino acids and said TAK1 amino acids is not more than about 0.5Å, and wherein at least one of said corresponding amino acids is not identical to the TAK1 amino acid to which it corresponds.

According to another embodiment of this invention is provided a machine-readable data storage medium, comprising a data storage material encoded with machine-readable data, wherein said data comprises all or part of a TAB1-binding pocket defined by structure coordinates of TAK1 amino acids A127, M131, P256, 1259, Y290 and F291 according to FIG. 1; or a molecule or molecular complex comprising all or part of a TAK1-like TAB1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a molecule or molecular complex comprising all or part of a TAK1-like ATP-binding pocket defined by structure coordinates of a set of corresponding amino acids, wherein the root mean square deviation of the backbone atoms between said set of corresponding amino acids and said TAK1 amino acids is not more than about 1.1, 0.9, 0.7 or 0.5Å, and wherein at least one of said corresponding amino acids is not identical to the TAK1 amino acid to which it corresponds.

In other embodiments of this invention is provided a machine-readable data storage medium, comprising a data storage material encoded with machine-readable data, wherein said data comprises all or part of any molecule or molecular complex disclosed in the above paragraphs.

In one embodiment of this invention is provided a computer comprising:

a machine-readable data storage medium, comprising a data storage material encoded with machine-readable data, wherein said data comprises all or part of a TAB1-binding pocket defined by structure coordinates of TAK1 amino acids A127, M131, P256, I259, Y290 and F291, according to FIG. 1; or a molecule or molecular complex comprising all or part of a TAK1-like TAB1-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å, or 1.0Å; or a molecule or molecular complex comprising all or part of a TAK1-like ATP-binding pocket defined by structure coordinates of a set of corresponding amino acids, wherein the root mean square deviation of the backbone atoms between said set of corresponding amino acids and said TAK1 amino acids is not more than about 0.5Å, and wherein at least one of said corresponding amino acids is not identical to the TAK1 amino acid to which it corresponds.

In other embodiments of this invention is provided a computer comprising:

a machine-readable data storage medium, comprising a data storage material encoded with machine-readable data, wherein said data comprises all or part of any molecule or molecular complex disclosed in the above paragraphs.

In one embodiment, a computer according to this invention comprises a working memory for storing instructions for processing the machine-readable data, a central-processing unit coupled to the working memory and to said machine-readable data storage medium for processing said machine-readable data into the three-dimensional structure. In one embodiment, the computer further comprises a display for displaying the three-dimensional structure as a graphical representation. In another embodiment, the computer further comprises commercially available software program to display the graphical representation. Examples of software programs include but are not limited to QUANTA [Molecular Simulations, Inc., San Diego, Calif. ©1998,2000], O [Jones et al., Acta Cryst. A, 47, pp. 110-119 (1991)] and RIBBONS [M. Carson, J. Appl. Cryst., 24, pp. 958-961 (1991)], which are incorporated herein by reference.

This invention also provides a computer comprising:

a) a machine-readable data storage medium comprising a data storage material encoded with machine-readable data, wherein the data defines any one of the above binding pockets or protein of the molecule or molecular complex;
b) a working memory for storing instructions for processing said machine-readable data;
c) a central processing unit (CPU) coupled to the working memory and to the machine-readable data storage medium for processing said machine readable data as well as an instruction or set of instructions for generating three-dimensional structural information of said binding pocket or protein; and
d) output hardware coupled to the CPU for outputting three-dimensional structural information of the binding pocket or protein, or information produced by using the three-dimensional structural information of said binding pocket or protein. The output hardware may include monitors, touchscreens, printers, facsimile machines, modems, disk drives, CD-ROMs, etc.

In the above embodiment, the outputting involves three-dimensional structural information. A computer of this invention may also be adapted to output other information or results. For example, a list of test compounds or potential inhibitor compounds may be outputted.

Three-dimensional data generation may be provided by an instruction or set of instructions such as a computer program or commands for generating a three-dimensional structure or graphical representation from structure coordinates, or by subtracting distances between atoms, calculating chemical energies for a TAK1 molecule or molecular complex or homologues thereof, or calculating or minimizing energies for an association of a TAK1 molecule or molecular complex or homologues thereof to a chemical entity. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through display terminals, touchscreens, printers, modems, facsimile machines, CD-ROMs or disk drives. The information can be in graphical or alphanumeric form.

FIG. 8 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bi-directional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding pocket of this invention using a program such as QUANTA [Molecular Simulations, Inc., San Diego, Calif. 01998,2000] as described herein. Output hardware might also include a printer 42, so that hard copy output may be produced, or a disk drive 24, to store system output for later use. Output hardware may also include a display terminal, a CD or DVD recorder, ZIP™ or JAZ™ drive, or other machine-readable data storage device.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are addressed in reference to the computational methods of drug discovery as described herein. Specific references to components of the hardware system 10 are included as appropriate throughout the following description of the data storage medium.

FIG. 9 shows a cross section of a magnetic data storage medium 100 which can be encoded with a machine-readable data that can be carried out by a system such as system 10 of FIG. 10. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in a manner that may be conventional, machine readable data such as that described herein, for execution by a system such as system 10 of FIG. 8.

FIG. 10 shows a cross section of an optically-readable data storage medium 110 which also can be encoded with such a machine-readable data, or set of instructions, which can be carried out by a system such as system 10 of FIG. 7. Medium 110 can be a conventional compact disk read only memory (CD-ROM) or a rewritable medium such as a magneto-optical disk that is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

In one embodiment, the data defines the above-mentioned binding pockets by comprising the structure coordinates of said amino acid residues according to FIG. 1, 2 or 3.

To use the structure coordinates generated for TAK1 or TAK1 homologue, one of its binding pockets, motifs, domains, or portion thereof, it is at times necessary to convert them into a three-dimensional shape or to generate three-dimensional structural information from them. This is achieved through the use of commercially or publicly available software that is capable of generating a three-dimensional structure of molecules or portions thereof from a set of structure coordinates. In one embodiment, the three-dimensional structure may be displayed as a graphical representation.

Therefore, according to another embodiment, this invention provides a machine-readable data storage medium comprising a data storage material encoded with machine readable data. In one embodiment, a machine programmed with instructions for using said data, is capable of generating a three-dimensional structure of any of the molecule or molecular complexes, or binding pockets thereof, that are described herein.

In certain embodiment, this invention also provides a computer for producing a three-dimensional structure of:

a) a molecule or molecular complex
comprising all or part of a TAK1 ATP-binding pocket defined by structure coordinates of TAK1 amino acids V377, A389, V419, F435, E436, F437, M438, C442, L489 and S499, according to FIG. 1;
b) a molecule or molecular complex
comprising all or part of a TAK1-like ATP-binding pocket defined by structure coordinates of corresponding amino acids that are identical to said TAK1 amino acids, wherein the root mean square deviation of the backbone atoms between said corresponding amino acids and said TAK1 amino acids is not more than about 3.0 Å, 2.5Å, 2.0Å, 1.5Å or 1.0Å; or 0.5Å; and/or
c) a molecule or molecular complex
comprising all or part of a TAK1-like ATP-binding pocket defined by structure coordinates of a set of corresponding amino acids, wherein the root mean square deviation of the backbone atoms between said set of corresponding amino acids and said TAK1 amino acids is not more than about 0.6Å, 0.5Å or 0.4Å, and wherein at least one of said corresponding amino acids is not identical to the TAK1 amino acid to which it corresponds, comprising:
i) a machine-readable data storage medium, comprising a data storage material encoded with machine-readable data, wherein said data comprises all or part of a TAK1 ATP-binding pocket defined by structure coordinates of TAK1 amino acids V377, A389, V419, F435, E436, F437, M438, C442, L489 and S499, according to FIG. 1; all or part of a TAK1-like ATP-binding pocket defined by structure coordinates of corresponding amino acids that are identical to said TAK1 amino acids, wherein the root mean square deviation of the backbone atoms between said corresponding amino acids and said TAK1 amino acids is not more than about 3.0Å, 2.5Å, 2.0Å, 1.5Å or 1.0Å; or all or part of a TAK1-like ATP-binding pocket defined by structure coordinates of a set of corresponding amino acids, wherein the root mean square deviation of the backbone atoms between said set of corresponding amino acids and said TAK1 amino acids is not more than about 0.6Å, 0.5Å or 0.4Å, and wherein at least one of said corresponding amino acids is not identical to the TAK1 amino acid to which it corresponds; and
ii) instructions for processing said machine-readable data into said three-dimensional structure.

According to other embodiments, the computer is also for producing the three-dimensional structure of the aforementioned molecules and molecular complexes and comprises the corresponding machine-readable data storage mediums. In one embodiment, the three-dimensional structure is displayed as a graphical representation.

In one embodiment, the structure coordinates of said molecules or molecular complexes are produced by homology modeling of at least a portion of the structure coordinates of FIG. 1 or 2 . Homology modeling can be used to generate structural models of TAK1 homologues or other homologous proteins based on the known structure of TAK1. This can be achieved by performing one or more of the following steps: performing sequence alignment between the amino acid sequence of an unknown molecule against the amino acid sequence of TAK1; identifying conserved and variable regions by sequence or structure; generating structure co-ordinates for structurally conserved residues of the unknown structure from those of TAK1; generating conformations for the structurally variable residues in the unknown structure; replacing the non-conserved residues of TAK1 with residues in the unknown structure; building side chain conformations; and refining and/or evaluating the unknown structure.

For example, since the protein sequence of the catalytic domains of TAK1 and homologues thereof can be aligned relative to each other, it is possible to construct models of the structures of TAK1 homologues, particularly in the regions of the active site, using the TAK1 structure. Software programs that are useful in homology modeling include XALIGN [Wishart, D. S. et al., Comput. Appl. Biosci., 10, pp. 687-88 (1994)] and CLUSTAL W Alignment Tool [Higgins D. G. et al., Methods Enzymol, 266, pp. 383-402 (1996)]. See also, U.S. Pat. No. 5,884,230. These references are incorporated herein by reference.

To perform the sequence alignment, programs such as the “bestfit” program available from the Genetics Computer Group [Waterman in Advances in Applied Mathematics 2, 482 (1981), which is incorporated herein by reference] and CLUSTAL W Alignment Tool [Higgins D. G. et al., Methods Enzymol, 266, pp. 383-402 (1996), which is incorporated by reference] can be used. To model the amino acid side chains of homologous TAK1 proteins, the amino acid residues in TAK1 can be replaced, using a computer graphics program such as “O” [Jones et al, (1991) Acta Cryst. Sect. A, 47: 110-119], by those of the homologous protein, where they differ. The same orientation or a different orientation of the amino acid can be used. Insertions and deletions of amino acid residues may be necessary where gaps occur in the sequence alignment.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol, 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three dimensional structure of the above molecules or molecular complexes (e.g., TAK1, homologues and portions thereof), or binding pockets thereof, can be stored in a machine-readable storage medium, which is capable of displaying three-dimensional structural information or a graphical three-dimensional representation of the structure.

### Rational Drug Design

The TAK1 structure coordinates or the three-dimensional graphical representation generated from these coordinates may be used in conjunction with a computer for a variety of purposes, including drug discovery. Accordingly, this invention allows for a method for structure based drug design comprising the applying the coordinates disclosed herein to identify TAK1 inhibitors. In certain embodiments, the computer is programmed with software to translate those coordinates into the three-dimensional structure of TAK1.

For example, the structure encoded by the data may be computationally evaluated for its ability to associate with chemical entities. Chemical entities that associate with TAK1 may inhibit or activate TAK1 or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention provides a method for designing, selecting and/or optimizing a chemical entity that binds to the molecule or molecular complex comprising the steps of:

(a) providing the structure coordinates of said molecule or molecular complex on a computer comprising the means for generating three-dimensional structural information from said structure coordinates; and
(b) designing, selecting and/or optimizing said chemical entity by employing means for performing a fitting operation between said chemical entity and said three-dimensional structural information of said molecule or molecular complex.

Three-dimensional structural information in step (a) may be generated by instructions such as a computer program or commands that can generate a three-dimensional structure or graphical representation; subtract distances between atoms; calculate chemical energies for a TAK1 molecule, molecular complex or homologues thereof; or calculate or minimize energies of an association of TAK1 molecule, molecular complex or homologues thereof to a chemical entity. These types of computer programs are known in the art. The graphical representation can be generated or displayed by commercially available software programs. Examples of software programs include but are not limited to QUANTA [Accelrys ©2001, 2002], O [Jones et al., Acta Crystallogr. A47, pp. 110-119 (1991)] and RIBBONS [Carson, J. Appl. Crystallogr., 24, pp. 9589-961 (1991)], which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

Another embodiment of the invention provides a method for evaluating the potential of a chemical entity to associate with the molecule or molecular complex as described previously. This evaluating may be for the purposes of optimizing or minimizing associating with a TAK1 protein.

This method comprises the steps of: a) employing computational means to perform a fitting operation between the chemical entity and the molecule or molecular complex described before; b) analyzing the results of said fitting operation to quantify the association between the chemical entity and the molecule or molecular complex; and, optionally, c) outputting said quantified association to a suitable output hardware, such as a CRT display terminal, a printer, a CD or DVD recorder, ZIP™ or JAZ™ drive, a disk drive, or other machine-readable data storage device, as described previously. The method may further comprise generating a three-dimensional structure, graphical representation thereof, or both, of the molecule or molecular complex prior to step a). In one embodiment, the method is for evaluating the ability of a chemical entity to associate with the binding pocket of a molecule or molecular complex.

In another embodiment, the method comprises the steps of:

a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a TAK1 protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, thereby evaluating the ability of said chemical entity to associate with said binding pocket.

In another embodiment, the invention provides a method of using a computer for evaluating the ability of a chemical entity to associate with the molecule or molecular complex, wherein said computer comprises a machine-readable data storage medium comprising a data storage material encoded with said structure coordinates defining said binding pocket and means for generating a three-dimensional graphical representation of the binding pocket, and wherein said method comprises the steps of:

(a) positioning a first chemical entity within all or part of said binding pocket using a graphical three-dimensional representation of the structure of the chemical entity and the binding pocket;
(b) performing a fitting operation between said chemical entity and said binding pocket by employing computational means;
(c) analyzing the results of said fitting operation to quantitate the association between said chemical entity and all or part of the binding pocket; and
(d) outputting said quantitated association to a suitable output hardware.

The above method may further comprise the steps of:

(e) repeating steps (a) through (d) with a second chemical entity; and
(f) selecting at least one of said first or second chemical entity that associates with said all or part of said binding pocket based on said quantitated association of said first or second chemical entity.

Alternatively, the structure coordinates of the TAK1 binding pockets may be utilized in a method for identifying an agonist or antagonist of a molecule comprising a binding pocket of TAK1. In certain embodiments, the method comprises steps of:

a) using a three-dimensional structure of the molecule or molecular complex to design, select or optimize a chemical entity;
b) contacting the chemical entity with the molecule or molecular complex;
c) monitoring the catalytic activity of the molecule or molecular complex; and
d) classifying the chemical entity as an agonist or antagonist based on the effect of the chemical entity on the catalytic activity of the molecule or molecular complex.

In one embodiment, step a) is performed using a graphical representation of the binding pocket or portion thereof of the molecule or molecular complex.

In one embodiment, the three-dimensional structure is displayed as a graphical representation.

In another embodiment, the method comprises the steps of:

a) constructing a computer model of a binding pocket of the molecule or molecular complex;
b) selecting a chemical entity to be evaluated by a method selected from the group consisting of assembling said chemical entity; selecting a chemical entity from a small molecule database; de novo ligand design of said chemical entity; and modifying a known agonist or inhibitor, or a portion thereof, of a TAK1 protein or homologue thereof;
c) employing computational means to perform a fitting program operation between computer models of said chemical entity to be evaluated and said binding pocket in order to provide an energy-minimized configuration of said chemical entity in the binding pocket; and
d) evaluating the results of said fitting operation to quantify the association between said chemical entity and the binding pocket model, thereby evaluating the ability of said chemical entity to associate with said binding pocket;
e) synthesizing said chemical entity; and
f) contacting said chemical entity with said molecule or molecular complex to determine the ability of said compound to activate or inhibit said molecule.

In another embodiment is provided a method of using a computer for selecting an orientation of a chemical entity that interacts favorably with a binding pocket or domain comprising amino acid residues selected from the group consisting of:

(i) a set of amino acid residues which are identical to TAK1 amino acid residues E105 and A107 as disclosed herein, wherein the root mean square deviation of the backbone atoms between the set of amino acid residues and the TAK1 amino acid residues which are identical is not greater than about 1.5Å; said method comprising the steps of:
(a) providing the structure coordinates of said binding pocket, domain or complex thereof on a computer comprising means for generating three-dimensional structural information from said structure coordinates;
(b) employing computational means to dock a first chemical entity in all or part of the binding pocket or domain;
(c) quantifying the association between said chemical entity and all or part of the binding pocket or domain for different orientations of the chemical entity; and
(d) selecting the orientation of the chemical entity with the most favorable interaction based on said quantified association. This method optionally further comprises the step of generating a three-dimensional graphical representation of the binding pocket or domain prior to step (b). The method also optionally comprises. This method optionally employs energy minimization, molecular dynamics simulations, or rigid-body minimizations are performed simultaneously with or following step (b). This method optionally further comprises the steps of: (e) repeating steps (b) through (d) with a second chemical entity; and (f) selecting at least one of said first or second chemical entity that interacts more favorably with said binding pocket or domain based on said quantified association of said first or second chemical entity.

In another embodiment, this invention provides a method of using a computer for selecting an orientation of a chemical entity with a favorable shape complementarity in a binding pocket comprising amino acid residues selected from the group consisting of: (i) a set of amino acid residues which are identical to TAK1 amino acid residues E105 and A107 as disclosed herein, wherein the root mean square deviation of the backbone atoms between the set of amino acid residues and the TAK1 amino acid residues which are identical is not greater than about 1.5Å; said method comprising the steps of:

(a) providing the structure coordinates of said binding pocket and all or part of the ligand bound therein on a computer comprising the means for generating three-dimensional structural information from said structure coordinates;
(b) employing computational means to dock a first chemical entity in all or part of the binding pocket;
(c) quantitating the contact score of said chemical entity in different orientations in the binding pocket; and
(d) selecting an orientation with the highest contact score. This method optionally comprises the further step of generating a three-dimensional graphical representation of all or part of the binding pocket and all or part of the ligand bound therein prior to step (b)

This method optionally comprises the further steps of (e) repeating steps (b) through (d) with a second chemical entity; and (f) selecting at least one of said first or second chemical entity that has a higher contact score based on said quantitated contact score of said first or second chemical entity.

This invention also provides a method of designing a compound or complex that interacts with a binding pocket or domain comprising amino acid residues selected from the group consisting of:

(i) a set of amino acid residues which are identical to TAK1 amino acid residues E105 and A107 as disclosed herein, wherein the root mean square deviation of the backbone atoms between the set of amino acid residues and the TAK1 amino acid residues which are identical is not greater than about 1.5Å; comprising the steps of:
(a) providing the structure coordinates of said binding pocket or domain on a computer comprising the means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the association between the first or second chemical entity and part of the binding pocket or domain;
(e) repeating steps (b) to (d) with another first and second chemical entity;
(f) selecting a first and a second chemical entity based on said quantified association of both of said first and second chemical entity;
(g) optionally, visually inspecting the relationship of the selected first and second chemical entity to each other in relation to the binding pocket or domain on a computer screen using the three-dimensional graphical representation of the binding pocket or domain and said first and second chemical entity; and
(h) assembling the selected first and second chemical entity into a compound or complex that interacts with said binding pocket or domain by model building.

This invention also provides a method of a method for identifying a candidate inhibitor that interacts with a binding site of a TAK1 protein or TAK1 kinase domain, or a homologue thereof, comprising the steps of:

(a) obtaining a crystal comprising a TAK1 protein or TAK1 kinase domain, or homologue thereof;
(b) obtaining the structure coordinates of amino acids of the crystal of step (a);
(c) generating a three-dimensional structure of the a TAK1 protein or TAK1 kinase domain or homologue thereof using the structure coordinates of the amino acids obtained in step (b) with a root mean square deviation from backbone atoms of said amino acids of not more than ±3.0Å;
(d) determining a binding site of the a TAK1 protein or TAK1 kinase domain or homologue thereof from said three-dimensional structure; and
(e) performing docking to identify the candidate inhibitor which interacts with said binding site. This method optionally further comprises the step of:
(f) contacting the identified candidate inhibitor with the a TAK1 protein or TAK1 kinase domain or homologue thereof in order to determine the effect of the inhibitor on catalytic activity. In certain embodiments, the binding site of the TAK1 kinase domain or homologue thereof determined in step (d) comprises the structure coordinates of E105 and A107 according to this invention, wherein the root mean square deviation from the backbone atoms of said amino acids is not more than ±1.5Å. In other embodiments, the binding site is any of the binding pockets defined herein.

Also provided is a method for identifying a candidate inhibitor that interacts with a binding site of a TAK1 kinase domain, or homologue thereof, comprising the step of determining a binding site from a three-dimensional structure of the TAK1 kinase domain or homologue thereof to design or identify the candidate inhibitor which interacts with said binding site.

Also provided is a method for identifying a candidate inhibitor of a molecule or molecular complex comprising a binding pocket or domain comprising amino acid residues selected from the group consisting of:
(i) a set of amino acid residues which are identical to TAK1 amino acid residues E105 and A107 as disclosed herein, wherein the root mean square deviation of the backbone atoms between said set of amino acid residues and said TAK1 amino acid residues which are identical is not greater than about 1.5Å; and
(ii) a set of amino acid residues which are identical to TAK1 amino acid residues V42, V90, M104, E105, A107, and as disclosed herein, wherein the root mean square deviation of the backbone atoms between said set of amino acid residues and said TAK1 amino acid residues which are identical is not greater than about 1.5Å; comprising the steps of:
(a) using a three-dimensional structure of all or part of the binding pocket or domain to design, select or optimize a plurality of chemical entities; and
(b) selecting said candidate inhibitor based on the inhibitory effect of said chemical entities on the catalytic activity of the molecule or molecular complex.

Also provided is a method of using a crystal according to any one of the embodiments herein in an inhibitor screening assay comprising:

(a) selecting a potential inhibitor by performing rational drug design with a three-dimensional structure determined for the crystal, wherein said selecting is performed in conjunction with computer modeling;
(b) contacting the potential inhibitor with a kinase; and
(c) detecting the ability of the potential inhibitor to inhibit the kinase.

Also provided is a method for identifying a potential inhibitor of a kinase comprising:

a) selecting or designing a potential inhibitor by performing rational drug design with a computer readable data storage material encoded with computer readable data comprising structure coordinates disclosed herein, wherein said selecting is performed in conjunction with computer modeling;
b) contacting the potential inhibitor with a kinase; and
c) detecting the ability of the potential inhibitor for inhibiting the kinase.

Also provided is a method for performing iterative drug design comprising crystallizing a TAK1 protein according to the method disclosed herein.

Also provided is a method for identifying an agent that interacts with an active site of a TAK1 (including a homologue or complex thereof), comprising the steps of:
a) obtaining a crystallized complex comprising TAK1;
b) obtaining the structural coordinates of amino acids of the crystallized complex of step a), wherein the structural coordinates are set forth herein;
c) generating a three dimensional model of TAK1 using the structural coordinates of the amino acids generated in step b) +/−a root mean square deviation from the backbone atoms of said amino acids of not more than 1.5Å;
d) determining an active site of the TAK1 from the three dimensional model; and
e) performing computer fitting analysis to identify an agent which interacts with the active site.

In another embodiment, this invention provides a method of identifying a TAK1 binding compound or a TAK1 inhibitor, comprising the step of using a three-dimensional structural representation of TAK1 or a fragment thereof comprising a TAK1 ATP-binding site or a TAK1 TAB1-binding site, to computationally screen a candidate compound for an ability to bind the TAK1 ATP-binding site or a TAK1 TAB1-binding site, respectively. In another embodiment, this invention provides a method of identifying a TAK1 binding compound or a TAK1 inhibitor comprising the step of using a three-dimensional structural representation of TAK1, or a fragment thereof comprising a TAK1 ATP-binding site or a TAK1 TAB 1-binding site, to computationally design a synthesizable candidate compound that binds or inhibits TAK1.

Any methods of this invention optionally comprise the steps of: synthesizing or otherwise obtaining the candidate compound; and testing the candidate compound for TAK1 binding activity or inhibitory activity.

Any methods of this invention optionally comprise a step of computationally design a binding compound comprises the steps of: identifying chemical entities or fragments capable of associating with the TAK1 ATP-binding site or a TAK1 TAB1-binding site; and assembling the chemical entities or fragments into a single molecule to provide the structure of the candidate compound.

In a specific embodiment, this invention provides a method of identifying a TAK1 binding compound or a TAK1 inhibitor, comprising the steps of (a) using a three-dimensional structural representation of TAK1, or a fragment thereof comprising a TAK1 ATP-binding site or a TAK1 TAB 1-binding site, to computationally screen a candidate compound for an ability to bind the TAK1 ATP-binding site or a TAK1 TAB1-binding site, (b) synthesizing the candidate compound; and (c) screening the candidate compound for TAK1 binding activity or TAK1 inhibitory activity, wherein the structural information comprises the atomic structure coordinates of residues comprising a TAK1 ATP-binding site or a TAK1 TAB 1-binding site.

In any embodiment of this invention, the three-dimensional structural representation comprises the three-dimensional structure defined by atomic structure coordinates according to FIG. 1 or FIG. 2

In another embodiment, this invention provides a method for designing an agent that I-nteracts with TAK1, comprising: providing a composition including TAK1; generating a three dimensional model of TAK1; and utilizing the three dimensional model to design an agent that interacts with TAK1, wherein the three dimensional model of TAK-1 includes relative structural coordinates of a plurality of atoms of TAK1, and the relative structural coordinates are selected according to: FIG. 1, ± a root mean square deviation from the backbone atoms of amino acids of not more than 1.5Å; or FIG. 2, ± a root mean square deviation from the backbone atoms of amino acids of not more than 1.5Å. In this embodiment, the three dimensional model optionally includes an agent that interact with TAK1. In one aspect, the method and the the three dimensional model is used to alter the chemical structure of the agent. This method optionally further comprises synthesizing or obtaining an agent and/or contacting the agent with TAK1 to determine the interaction between the agent and TAK1.

In another embodiment, the invention provides a method for designing an agent that interacts with TAK1, comprising: generating a three dimensional model of TAK1 including relative structural coordinates of a plurality of atoms of an active site of TAK1 and relative structural coordinates of a first agent that interacts with TAK1; utilizing the three dimensional model to design a second agent that interacts with TAK1, wherein utilizing includes altering the relative structural coordinates of the first agent; synthesizing or obtaining the second agent; and determining the interaction of TAK1 with the second agent, wherein the relative structural coordinates of TAK1 are selected according to: FIG. 1, ± a root mean square deviation from the backbone atoms of amino acids of not more than 1.5Å; or FIG. 2, ± a root mean square deviation from the backbone atoms of amino acids of not more than 1.5Å.

In this embodiment, the altering of the relative structural coordinates of the first agent includes adding, removing, or changing the position of an atom of the first agent. This method optionally further comprises comparing the model including relative structural coordinates of the first agent to a model including the second agent.

In another embodiment, the invention provides a method for designing an agent that interacts with TAK1, comprising: generating a three dimensional model of TAK1; utilizing the three dimensional model to design an agent that interacts with TAK1; and synthesizing or obtaining the agent, wherein the three dimensional model of TAK1 includes relative structural coordinates of a plurality of atoms of TAK1, and the relative structural coordinates are selected according to: FIG. 1, ± a root mean square deviation from the backbone atoms of amino acids of not more than 1.5Å; or FIG. 2, ± a root mean square deviation from the backbone atoms of amino acids of not more than 1.5Å. In this embodiment, the three dimensional model optionally includes the agent and the method can involve utilizing the three dimensional model to alter the chemical structure of the agent. Further, this embodiment, optionally further comprises providing a composition including TAK1.

In embodiments involving a composition, the composition optionally includes a crystal including TAK1 or the composition includes an isotopically labeled TAK1 and the method optionally comprises determining the relative structural coordinates of atoms of TAK1 from the composition.

In one aspect, this invention involves utilizing the three dimensional includes designing an agent that interacts more strongly with TAK1 than with another kinase. In embodiments involving an agent, the agent is an agent designed by a structure based drug design method. In certain embodiments, the method optionally further comprises contacting the agent with TAK1 to determine the interaction between the agent and TAK1.

A would be recognized, in a method of this invention, a three dimensional model may be used to determine the fit of an agent with an active site of TAK1. In any embodiment of this invention, a three dimensional model may be used to identify residues of TAK1 that can influence the interaction of an agent with TAK1. In another aspect, this invention involves comparing a three dimensional model of TAK1 to another kinase.

In any of these methods, the relative structural coordinates include relative structural coordinates of an atom of TAK1 binding pocket, more specifically an ATP-binding pocket or a TAB1-binding pocket of TAK1. In specific embodiments, the ATP-binding pocket and a TAB1-binding pocket of TAK1 are as defined herein.

In another embodiment, this invention provides a method of identifying a compound that binds to a TAK1 binding site, said method comprising: modeling a test compound that fits spatially into the TAK1 binding site using an atomic structural model of the TAK1 binding site or portion thereof; and screening said test compound in an assay that measures binding of the test compound to the TAK1 binding site, thereby identifying a test compound that binds to the TAK1 binding site.

In certain embodiments, the TAK1 binding site is the ATP-binding site or the TAB 1-binding site.

In certain embodiments, the atomic structural model is a model of human TAK1 and comprises atomic coordinates of amino acid residues selected from the group consisting those amino acids as shown in FIG. 1 or FIG. 2. In a more specific embodiment, the atomic structural model is a model of human TAK1 ATP-binding site or the TAB1-binding site and comprises atomic coordinates of amino acid residues selected from the group consisting those amino acids of the ATP-binding site or the TAB1-binding site as disclosed herein according to FIG. 1 or FIG. 2.

This invention includes atomic structural models wherein data which is experimentally derived. As would be recognized, in computer-aided drug design methods of this invention, the atomic structural model is provided to a computerized modeling system.

In certain embodiments, the assay is in vitro. In other embodiments, the assay is in vivo. As would be recognized, screening can include high throughput screening. Test compounds may be obtained by any means (e.g., synthses or purchase) and the test compound can be from a library of compounds. The test compound is an agonist or antagonist of TAK1 binding. Ideally, the test compound is an inhibitor of TAK1.

In certain embodiments, the test compound interacts with one or more amino acid residue of the TAK1 ATP-binding pocket or the TAK1 TAB-1-binding pocket. Nothing herein limits the structure of the test compound. In certain embodiments, the test compound is a small organic molecule, a peptide, or a peptidomimetic, with small organic molecules being preferred as test compounds for the ATP-binding pocket and small organic molecules or peptidomimetic being preferred as test compounds for the TAB1-binding site.

In any embodiment of this invention involving a crystal, the crystal is optionally a TAK1 kinase domain bound to an active site inhibitor. In certain embodiments, the crystal belongs to space group 1222 and has unit cell parameters of a=58.4, b=144.3, and c=134.7.

Any of the methods of this invention involving a binding pocket may employ a binding pocket defined by the TAK1-TAB1 chimera of FIG. 1 or FIG. 2. In preferred embodiments, a binding pocket employed in a method of this invention is one or more of the binding pockets defined herein.

For the first time, the present invention permits the use of molecular design techniques to identify, select and design chemical entities, including inhibitory compounds, capable of binding to TAK1 or TAK1-like binding pockets, motifs and domains. It should be understood that these chemical entities may be peptides, peptidomimetics, small organic compounds, or antibodies.

Applicants' elucidation of binding pockets on TAK1 provides the necessary information for designing new chemical entities and compounds that may interact with TAK1 or TAK1-like substrate or ATP-binding pockets, in whole or in part.

Throughout this description, disclosure about the ability of a chemical entity to bind to, associate with or inhibit TAK1 binding pockets refers to features of the entity alone. Assays to determine if a compound binds to TAK1 are well known in the art and are exemplified below.

The design of chemical entities that bind to or inhibit TAK1 binding pockets according to this invention generally involves consideration of two factors. First, the entity must be capable of physically and structurally associating with parts or all of the TAK1 binding pockets. Non-covalent molecular interactions important in this association include hydrogen bonding, van der Waals interactions, hydrophobic interactions and electrostatic interactions.

Second, the entity must be able to assume a conformation that allows it to associate with the TAK1 binding pockets directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the TAK1 or TAK1-like binding pockets.

The potential inhibitory or binding effect of a chemical entity on TAK1 binding pockets may be analyzed prior to its actual synthesis and testing by the use of computer modeling techniques. If the theoretical structure of the given entity suggests insufficient interaction and association between it and the TAK1 binding pockets, testing of the entity is obviated. However, if computer modeling indicates a strong interaction, the compound may then be synthesized and tested for its ability to bind to a TAK1 binding pocket. This may be achieved by testing the ability of the molecule to inhibit TAK1 using the assays described in Example 7. In this manner, synthesis of inoperative compounds may be avoided.

A potential inhibitor of a TAK1 binding pocket may be computationally evaluated by means of a series of steps in which chemical entities or fragments are screened and selected for their ability to associate with the TAK1 binding pockets.

One skilled in the art may use one of several methods to screen chemical entities or fragments for their ability to associate with a TAK1 binding pocket. This process may begin by visual inspection of, for example, a TAK1 binding pocket on the computer screen based on the TAK1 structure coordinates in FIG. 1 or 2 or other coordinates which define a similar shape generated from the machine-readable storage medium. Selected fragments or chemical entities may then be positioned in a variety of orientations, or docked, within that binding pocket as defined supra. Docking may be accomplished using software such as QUANTA and Sybyl [Tripos Associates, St. Louis, Mo.], followed by energy minimization and molecular dynamics with standard molecular mechanics force fields, such as CHARMM and AMBER.

Specialized computer programs may also assist in the process of selecting fragments or chemical entities. These include:

1. GRID [P. J. Goodford, “A Computational Procedure for Determining Energetically Favorable Binding Sites on Biologically Important Macromolecules”, J. Med. Chem, 28, pp. 849-857 (1985)]. GRID is available from Oxford University, Oxford, UK.

2. MCSS [A. Miranker et al., “Functionality Maps of Binding Sites: A Multiple Copy Simultaneous Search Method.” Proteins: Structure, Function and Genetics, 11, pp. 29-34 (1991)]. MCSS is available from Molecular Simulations, San Diego, Calif.

3. AUTODOCK [D. S. Goodsell et al., “Automated Docking of Substrates to Proteins by Simulated Annealing”, Proteins: Structure, Function, and Genetics, 8, pp. 195-202 (1990)]. AUTODOCK is available from Scripps Research Institute, La Jolla, Calif.

4. DOCK [I. D. Kuntz et al., “A Geometric Approach to Macromolecule-Ligand Interactions”, J. Mol. Biol., 161, pp. 269-288 (1982)]. DOCK is available from University of California, San Francisco, Calif.

Once suitable chemical entities or fragments have been selected, they can be assembled into a single compound or complex of compounds. Assembly may be preceded by visual inspection of the relationship of the fragments to each other on the three-dimensional image displayed on a computer screen in relation to the structure coordinates of TAK1. This would be followed by manual model building using software such as QUANTA or Sybyl [Tripos Associates, St. Louis, Mo.].

*-Useful programs to aid one of skill in the art in connecting the individual chemical entities or fragments include:

1. CAVEAT [P. A. Bartlett et al., “CAVEAT: A Program to Facilitate the Structure-Derived Design of Biologically Active Molecules”, in Molecular Recognition in Chemical and Biological Problems“, Special Pub., Royal Chem. Soc., 78, pp. 182-196 (1989); G. Lauri and P. A. Bartlett, “CAVEAT: a Program to Facilitate the Design of Organic Molecules”, J. Comput. Aided Mol. Des. , 8, pp. 51-66 (1994)]. CAVEAT is available from the University of California, Berkeley, Calif.

2. 3D Database systems such as ISIS (MDL Information Systems, San Leandro, Calif.). This area is reviewed in Y. C. Martin, “3D Database Searching in Drug Design”, J. Med. Chem., 35, pp. 2145-2154 (1992).

3. HOOK [M. B. Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994)]. HOOK is available from Molecular Simulations, San Diego, Calif.

Instead of proceeding to build an inhibitor of a TAK1 binding pocket in a step-wise fashion one fragment or chemical entity at a time as described above, inhibitory or other TAK1 binding compounds may be designed as a whole or “de novo” using either an empty binding pocket or optionally including some portion(s) of a known inhibitor(s). There are many de novo ligand design methods including:

1. LUDI [H.-J. Bohm, “The Computer Program LUDI: A New Method for the De Novo Design of Enzyme Inhibitors”, J. Comp. Aid. Molec. Design, 6, pp. 61-78 (1992)]. LUDI is available from Molecular Simulations Incorporated, San Diego, Calif.

2. LEGEND [Y. Nishibata et al., Tetrahedron, 47, p. 8985 (1991)]. LEGEND is available from Molecular Simulations Incorporated, San Diego, Calif.

3. LeapFrog [available from Tripos Associates, St. Louis, Mo.].

4. SPROUT [V. Gillet et al., “SPROUT: A Program for Structure Generation)”, J. Comput. Aided Mol. Design, 7, pp. 127-153 (1993)]. SPROUT is available from the University of Leeds, UK.

Other molecular modeling techniques may also be employed in accordance with this invention [see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a chemical entity has been designed or selected by the above methods, the efficiency with which that chemical entity may bind to a TAK1 binding pocket may be tested and optimized by computational evaluation. For example, an effective TAK1 binding pocket inhibitor must preferably demonstrate a relatively small difference in energy between its bound and free states (i.e., a small deformation energy of binding). Thus, the most efficient TAK1 binding pocket inhibitors should preferably be designed with a deformation energy of binding of not greater than about 10 kcal/mole, more preferably, not greater than 7 kcal/mole. TAK1 binding pocket inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to a TAK1 binding pocket may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C [M. J. Frisch, Gaussian, Inc., Pittsburgh, Pa. ©1995]; AMBER, version 4.1 [P. A. Kollman, University of California at San Francisco, 01995]; QUANTA/CHARMM [Accelrys, San Diego, Calif. A ©2001, 2002]; Insight II/Discover [Accelrys, San Diego, Calif. ©2001, 2002]; DelPhi [Accelrys, San Diego, Calif. ©2001, 2002]; and AMSOL [Quantum Chemistry Program Exchange, Indiana University]. These programs may be implemented, for instance, using a Silicon Graphics workstation such as an Indigo2 with “IMPACT” graphics. Other hardware systems and software packages will be known to those skilled in the art.

Another approach enabled by this invention, is the computational screening of small molecule databases for chemical entities or compounds that can bind in whole, or in part, to a TAK1 binding pocket. In this screening, the quality of fit of such entities to the binding pocket may be judged either by shape complementarity or by estimated interaction energy [E. C. Meng et al., J. Comp. Chem., 13, pp. 505-524 (1992)].

Another particularly useful drug design technique enabled by this invention is iterative drug design. Iterative drug design is a method for optimizing associations between a protein and a compound by determining and evaluating the three-dimensional structures of successive sets of protein/compound complexes.

According to another embodiment, the invention provides compounds which associate with a TAK1 binding pocket produced or identified by the method set forth above.

Another particularly useful drug design technique enabled by this invention is iterative drug design. Iterative drug design is a method for optimizing associations between a protein and a compound by determining and evaluating the three-dimensional structures of successive sets of protein/compound complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex.

### Structure Determination of Other Molecules

The structure coordinates set forth in FIG. 1 or 2 can also be used to aid in obtaining structural information about another crystallized molecule or molecular complex. This may be achieved by any of a number of well-known techniques, including molecular replacement.

According to an alternate embodiment, the machine-readable data storage medium comprises a data storage material encoded with a first set of machine readable data which comprises the Fourier transform of at least a portion of the structure coordinates set forth in FIG. 1 or 2 or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex, wherein said computer comprises:

a) a machine-readable data storage medium comprising a data storage material encoded with machine-readable data, wherein said data comprises at least a portion of the structural coordinates of TAK1 according to FIG. 1 or 2 or homology model thereof;
b) a machine-readable data storage medium comprising a data storage material encoded with machine-readable data, wherein said data comprises X-ray diffraction data obtained from said molecule or molecular complex; and
c) instructions for performing a Fourier transform of the machine readable data of (a) and for processing said machine readable data of (b) into structure coordinates.

For example, the Fourier transform of at least a portion of the structure coordinates set forth in FIG. 1 or 2 or homology model thereof may be used to determine at least a portion of the structure coordinates of TAK1 homologues.

Therefore, in another embodiment this invention provides a method of utilizing molecular replacement to obtain structural information about a molecule or molecular complex whose structure is unknown comprising the steps of:

a) crystallizing said molecule or molecular complex of unknown structure;
b) generating an X-ray diffraction pattern from said crystallized molecule or molecular complex;
c) applying at least a portion of the structure coordinates set forth in FIG. 1 or 2 or homology model thereof to the X-ray diffraction pattern to generate a three-dimensional electron density map of the molecule or molecular complex whose structure is unknown; and
d) generating a structural model of the molecule or molecular complex from the three-dimensional electron density map.

In one embodiment, the method is performed using a computer. In another embodiment, the molecule is selected from the group consisting of TAK1 and TAK1 homologues. In another embodiment, the molecule is a TAK1 molecular complex or homologue thereof.

By using molecular replacement, all or part of the structure coordinates of the TAK1 as provided by this invention (and set forth in FIG. 1 or 2) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that can not be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the TAK1 according to FIG. 1 or 2 or homology model thereof within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex [E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecule or molecular complex that is sufficiently homologous to any portion of the TAK1 can be resolved by this method.

In a preferred embodiment, the method of molecular replacement is utilized to obtain structural information about a TAK1 homologue. The structure coordinates of TAK1 as provided by this invention are particularly useful in solving the structure of TAK1 complexes that are bound by ligands, substrates and inhibitors.

Furthermore, the structure coordinates of TAK1 as provided by this invention are useful in solving the structure of TAK1 proteins that have amino acid substitutions, additions and/or deletions (referred to collectively as “TAK1 mutants”, as compared to naturally occurring TAK1). These TAK1 mutants may optionally be crystallized in co-complex with a chemical entity, such as a non-hydrolyzable ATP analog or a suicide substrate. The crystal structures of a series of such complexes may then be solved by molecular replacement and compared with that of wild-type TAK1. Potential sites for modification within the various binding pockets of the enzyme may thus be identified. This information provides an additional tool for determining the most efficient binding interactions, for example, increased hydrophobic interactions, between TAK1 and a chemical entity or compound.

The structure coordinates are also particularly useful in solving the structure of crystals of TAK1 or TAK1 homologues co-complexed with a variety of chemical entities. This approach enables the determination of the optimal sites for interaction between chemical entities, including candidate TAK1 inhibitors. For example, high resolution X-ray diffraction data collected from crystals exposed to different types of solvent allows the determination of where each type of solvent molecule resides. Small molecules that bind tightly to those sites can then be designed and synthesized and tested for their TAK1 inhibition activity.

All of the complexes referred to above may be studied using well-known X-ray diffraction techniques and may be refined versus 1.5-3.4A resolution X-ray data to an R value of about 0.30 or less using computer software, such as X-PLOR (Yale University, ©1992, distributed by Molecular Simulations, Inc.; see, e.g., Blundell & Johnson, supra; Meth. Enzymol., vol. 114 & 115, H. W. Wyckoff et al., eds., Academic Press (1985)), CNS (Brunger et al., Acta Crystallogr. D. Biol. Crystallogr., 54, pp. 905-921, (1998)) or CNX (Accelrys, ©2000,2001). This information may thus be used to optimize known TAK1 inhibitors, and more importantly, to design new TAK1 inhibitors.

In order that this invention be more fully understood, the following examples are set forth. These examples are for the purpose of illustration only and are not to be construed as limiting the scope of the invention in any way.

### EXAMPLE 1

Expression and Purification of TAK-TAB Constructs for Crystallography and Enzymology

The expression of TAK1 was carried out using standard procedures known in the art.

A truncated version of the TAK1 kinase domain (residues I31-Q303) fused to a 36-residue TAB1 segment (residues H468-P504) was cloned downstream of the polyhedrin promoter in the baculovirus donor vector, pBEV10TOPO, using the BamHI and EcoRI sites. The vector incorporated an N-terminal hexa-histidine purification tag and thrombin cleavage site. pBEV10TOPO is a Bac-to-Bac compatible vector and recombinant virus was generated according to the manufacturer's recommendations. These initially transfected Spodoptera frugiperda (Sf9) cells were tested for the expression of TAK1(I31-Q303)/TAB1(H468-P504) protein by loading a crude extract of the transfected insect cells onto an SDS-PAGE gel and immunoblot analysis using an anti-His (Sigma) antibody. Upon confirmation of the expression of the TAK1(I31-Q303)/TAB1(H468-P504) protein the virus was further amplified two times to obtain high titer stocks and used for optimisation of expression studies in Hi5 and Sf9 insect cells (1.5×10⁶ cells/ml) at 27° C. with shaking at 100 rpm, using defined volumes of virus. After infection, cells were harvested at regular intervals of 24, 48 and 72 hours and optimum expression was determined by SDS-PAGE gel and immunoblot analysis using an anti-His (Sigma) antibody. Large-scale cultivation/expression was conducted with Hi5 insect cells (1.5x10⁶ cells/m1) using a multiplicity of infection (M.O.I.) of between 2 and 5 of recombinant TAK1(I31-Q303)/TAB1(H468-P504) virus particles/cell, incubated at 27° C., 100 rpm and harvested 48 hours post-infection.

Cell pellets were resuspended into Lysis buffer (50 mM Hepes, pH 7.8, 250 mM NaCl, 5 mM β-mercaptoethanol, 10% Glycerol (v/v), 0.05% Tween, 5 mM Imidazole, 0.5 mM Sodium orthovanadate, 50 mM sodium fluoride, 10 mM β-glycerophosphate, Protease inhibitor Cocktail I and Phosphatase Inhibitor) and disrupted by dounce homogenization, on ice. Resuspended cells were further mechanically disrupted using a Microfluidizer (Microfluidics, Newton, Mass.). The cell debris was removed by centrifugation (21,000 rpm, 15 min at 4° C.) and the supernatant incubated for 2.5 hours at 4° C. with pre-equilibrated Nickel-NTA metal affinity resin. The NiNTA resin was collected by centrifugation (1000 g, 4 min) and the non-specifically bound protein was removed by washing with 30× bead volume of Lysis buffer. The TAK1(I31-Q303)/TAB(H468-P504) protein was eluted 3 times with Lysis buffer containing 200 mM Imidazole and a final elution was carried out using Lysis buffer containing 500 mM Imidazole. The N-terminal hexa-histidine tag was removed by a 4° C. overnight incubation using 20 Units thrombin (Sigma) per mg of eluted protein and successful cleavage analysed by SDS-PAGE gel. The cleaved protein was then isolated by size-exclusion on a Superdex 200(26/60) column (Amersham Biotech, Sweden) pre-equilibrated in Gel Filtration Buffer (50 mM Hepes, pH 7.8, 500 mM NaCl, 5 mM DTT and 10% Glycerol). Further purification of TAK1(I31-Q303)/TAB(H468-P504) was performed using anion exchange chromatography. Protein was applied to a 6 ml Resource Q column (Amersham Biotech, Sweden) pre-equilibrated with Buffer A (25 mM Tris, pH 8.0, 50 mM NaCl and 5 mM DTT) at a flow rate of 0.5 ml/min. Unbound protein was extensively washed with 10 CV of Buffer A. Bound protein was eluted with a linear salt gradient (0-15%) of Buffer B (25 mM Tris, pH 8.0, 1 M NaCl and 5 mM DTT) over 10 CV at a flow rate of 0.5 ml/min. A second gradient (15-100%) was applied over 2 CV to elute remaining proteins .The resultant protein fractions were analysed by SDS-PAGE gel and fractions containing purified TAK1(I31 Q303)/TAB1(H468-P504 were pooled accordingly. The purified protein sample was dialysed against 50 mM Hepes pH8.0 containing 200 mM NaCl, 10% glycerol and 5 mM DTT at 4° C. and concentrated to 10 mg/ml for crystallization. All proteins TAK-TAB chimera proteins were prepared using a similar protocol.

### EXAMPLE 2

### Enzymatic Characterization of Chimeric TAK-TAB Proteins

Activity of the TAK-1:TAB-1 fusion contructs was determined using a standard coupled enzyme system (Fox et al., Protein Sci.,. 7, pp. 2249 (1998)). Reactions were carried out in a solution containing 100 mM HEPES (pH 7.5), 10 mM MgCl₂, 25 mM NaCl, 2 mM DTT and 3% DMSO. Final peptidic substrate (full length Myelin Basic Protein, Vertex Pharmaceuticals Inc., Cambridge, Mass.) concentration in the assay was 15 mM. Reactions were carried out at 30° C. in the presence of 500 nM TAK-1:TAB1 construct and a titration of ATP (Sigma Chemicals, St Louis, Mo.) at final assay concentrations spanning 0 to 500 mM. Final concentrations of the components of the coupled enzyme system were 2.5 mM phosphoenolpyruvate, 300 mM NADH, 60 mg/ml pyruvate kinase and 20 mg/ml lactate dehydrogenase. An assay stock buffer solution was prepared containing all of the reagents listed above with the exception of ATP and DMSO. The assay stock buffer solution (60 ml) was incubated in a 96 well plate with 2 ml DMSO. The reaction was initiated by the addition of 5 ml of ATP (final assay concentrations spanning 0 to 500 mM). Rates of reaction were obtained using a Molecular Devices Spectramax plate reader (Sunnyvale, Calif.) over 10 min at 30° C. The ATP Km and Vmax values were determined from the rate data as a function of ATP concentration using computerized nonlinear regression (Prism 4.0a, Graphpad Software, San Diego, Calif.) .

### EXAMPLE 3

### Formation of TAK1—Inhibitor Complex for Crystallization

Crystals of TAK1—Inhibitor Complex Crystals were Formed by Co-Crystallizing the protein with the inhibitors or with adenosine. The inhibitor was added to the TAK1 protein solution immediately after the final protein concentration step (Example 1), immediately prior to setting up the crystallization drop.

### EXAMPLE 4

### Crystallization of TAK1 and TAK1—Inhibitor Complexes

Crystallization of TAK1 was carried out using the hanging drop vapor diffusion technique. The TAK1 formed lozenge-like crystals over a reservoir containing 600-800 mM sodium citrate, 200 mM sodium chloride, 100 mM Tris-HCl pH7.0 and 10 mM DTT. The crystallization droplet contained 0.25 μl of 10 mg ml-¹ protein solution and 0.25 μl of reservoir solution. Crystals formed in approximately 72 hours.

The formed crystals were transferred to a reservoir solution containing 15% ethylene glycol. After soaking the crystals in 15% ethylene glycol for less than 2 minutes, the crystals were scooped up with a cryo-loop, frozen in liquid nitrogen and stored for data collection.

### EXAMPLE 5

### Soaking of Preformed TAK1 Complex Crystals in Solutions of Other Inhibitors

An alternative method for preparing complex crystals of TAK1 is to remove a co-complex crystal grown by hanging drop vapour diffusion (Example 3) from the hanging drop and place it in a solution consisting of a reservoir solution containing 0.5 mM staurosporine or another inhibitor for a period of time between 1 and 24 hours.

The crystals can then be transferred to a reservoir solution containing 15% ethylene glycol and 0.5 mM staurosporine or another inhibitor. After soaking the crystal in this solution for less than 2 minutes, the crystals were scooped up with a cryo-loop, frozen in liquid nitrogen and stored for data collection. Subsequent data collection and structure determination (Example 5) reveals that inhibitors bound to the ATP-binding site of TAK1 can be exchanged for the TAK1 complex crystals.

### EXAMPLE 6

### X-Ray Data Collection and Structure Determination

The TAK1—inhibitor complex structures and the TAK1—adenosine structure were solved by molecular replacement using X-ray diffraction data collected either (i) at beam line 14.2 of the CCLRC Synchrotron Radiation Source, Daresbury, Cheshire, UK, or (ii) Vertex Pharmaceuticals (Europe) Ltd, 88 Milton Park, Abingdon, Oxfordshire OX14 4RY, UK. The diffraction images were processed with the program MOSFLM [A. G. Leslie, Acta Cryst. D, 55, pp. 1696-1702 (1999)] and the data was scaled using SCALA [Collaborative Computational Project, N., Acta Cryst. D, 50, pp. 760-763 (1994)].

The data statistics, unit cell parameters and spacegroup of the TAK1—adenosine crystal structure is given in Table 2. The starting phases for the TAK1 complexes were obtained by molecular replacement using coordinates of Aurora-2 as a search model in the program AMoRe [J. Navaza, Acta. Cryst. A, 50, pp. 157-163 (1994)]. The asymmetric unit contained a single TAK1 complex. Multiple rounds of rebuilding with QUANTA [Molecular Simulations, Inc., San Diego, Calif. ©1998,2000] and refinement with CNX [Accelrys Inc., San Diego, Calif. ©2000] resulted in a final model that included residues 31 to 178 and 191 to 303 of TAK1 and 468 to 495 of TAB1. The refined model has a crystallographic R-factor of 21.2% and R-free of 23.1%.

The data statistics, unit cell parameters and spacegroup of the TAK1—3-[6-(4-Acetyl-3,5-dimethyl-piperazin-1-yl)-pyridin-2-yl]-1H-pyrrolo [2,3-b]pyridine-5-carboxylic acid methyl ester crystal structure is given in Table 3. The starting phases were obtained by molecular replacement using coordinates of the TAK1-adenosinecomplex as a search model in the program AMoRe. Multiple rounds of rebuilding with QUANTA [Molecular Simulations, Inc., San Diego, Calif. ©1998,2000] and refinement with CNX [Accelrys Inc., San Diego, Calif. ©2000] resulted in a final model that included residues 31 to 178 and 191 to 303 of TAK1 and 468 to 495 of TAB1. The refined model has a crystallographic R-factor of 27.8% and R-free of 32.1%.

In the above models, disordered residues were not included in the model. Alanine or glycine residues were used in the model if the side chains of certain residues could not be located in the electron density.

### EXAMPLE 7

### Overall Structure of the TAK1-TAB1 Chimera

TAK1 has the typical bi-lobal catalytic kinase fold or structural domain [S. K. Hanks, et al., Science, 241, pp. 42-52 (1988); Hanks, S. K. and A. M. Quinn, Meth. Enzymol., 200, pp. 38-62 (1991)] with a β-strand sub-domain (residues 31-104) at the N-terminal end and an a-helical sub-domain at the C-terminal end (residues 112-303) (FIG. 3). The ATP-binding pocket is at the interface of the a-helical and I3-strand domains, and is bordered by the glycine rich loop and the hinge. The activation loop is disorder in both crystal structures.

Comparison with other kinases such as LCK , p38 and Aurora2 revealed that the structure of TAK1 resembles closely the substrate-bound, activated, form of a kinase. The overall topology of the kinase domain is similar to other serine/threonine kinases and several other tyrosine kinases, particularly LCK, ITK and Aurora-2, and distinct from other members of the MAP kinase family (P38 and MK2; Table 1).

### EXAMPLE 8

### Catalytic Active Site of TAK1—Inhibitor Complexes

The inhibitor 3- [6-(4-Acetyl-3,5-dimethyl-piperazin-1-yl)-pyridin-2-yl]-1H-pyrrolo[2,3-b]pyridine-5 -carboxylic acid methyl ester is bound in the deep cleft of the catalytic active site in the TAK1 structure. The inhibitor forms two hydrogen bonds with the hinge portion of the ATP-binding pocket (dotted lines).

The side chains of D175 and K63 are positioned inside the ATP-binding pocket and make a salt-bridge interaction with each other. Like other kinases, K63 and D175 are catalytically important residues and resemble a catalytically active conformation.

### EXAMPLE 9

### The Use of TAK1 Coordinates for Inhibitor Design

The coordinates of FIG. 1 or 2 are used to design compounds, including inhibitory compounds, that associate with TAK1 or homologues of TAK1. This process may be aided by using a computer comprising a machine-readable data storage medium encoded with a set of machine-executable instructions, wherein the recorded instructions are capable of displaying a three-dimensional representation of the TAK1 or a portion thereof. The graphical representation is used according to the methods described herein to design compounds. Such compounds associate with the TAK1 at the ATP-binding pocket, substrate binding pocket or TAB 1 binding pocket.

### EXAMPLE 10

### The Use of TAK1 Coordinates in the Design of TAK1-Specific Antibodies

The atomic coordinates in FIG. 1 or 2 also define, in great detail, the external solvent-accessible, hydrophilic, and mobile surface regions of the TAK1 catalytic kinase domain. Anti-peptide antibodies are known to react strongly against highly mobile regions but do not react with well-ordered regions of proteins. Mobility is therefore a major factor in the recognition of proteins by anti-peptide antibodies [J. A. Tainer et al., Nature, 312, pp. 127-134 (1984)]

One skilled in the art would therefore be able to use the X-ray crystallography data to determine possible antigenic sites in the TAK1 kinase domain. Possible antigenic sites are exposed, small and mobile regions on the kinase surface which have atomic B-factors of greater than 80Å² in FIGS. 1 and 2. This information can be used in conjunction with data from immunological studies to design and produce specific monoclonal or polyclonal antibodies.

This process may be aided by using a computer comprising a machine-readable data storage medium encoded with a set of machine-executable instructions, wherein the recorded instructions are capable of displaying a three-dimensional representation of the TAK1 or a portion thereof

### EXAMPLE 11

### Enzymatic Investigation of TAK1-TAB1 Chimeras

Many studies have attempted to further elucidate the molecular mechanisms that regulate the activation of Tak1 and more specifically the role that Tab 1 plays in the process. The activity of Tak1 is dependent on a series of Tak1 catalysed intramolecular phosphorylation events mapped to three residues on Tak1 (Thr184, Thr187, Ser192) (ref 15, 16, 17) along with as yet unmapped phosphorylation sites on Tab1 (Sakurai 2000).

We have prepared and analysed a number of Tak1-Tab1 fusion proteins to explore the effect that varied truncations in both Tak1 and Tab1 had on substrate kinetics. The purified recombinant fusion protein first described by Sakurai et al (Table1) shows a high affinity for ATP with Km of 24±μM and moderate kinase activity with kcat of 7.2 min⁻¹. Truncation of the TAB1 region to just 36 residues had no significant effect on the substrate kinetics with the purified recombinant protein showing a Km of 21±1 μM and kcat of 11.4 min⁻¹, contrasting with data for co-expression of Tab1 and Tak1 in mammalian cells, which showed differences in enzyme activity (15). Our data shows that varying the length of the Tab1 peptide has no direct effect on either ATP binding affinity or enzyme rate. These differences might arise from enhanced stability of cellular TAK-TAB complexes.

We then examined the role of the Tak1 N-terminus on activity by characterising the kinetics of a truncated fusion protein consisting of the Tak1 residues 31-301 and the short 37-residue Tab1 peptide. No significant differences were observed in either substrate affinity (Km 27 μM) or enzyme turnover (kcat 15min⁻¹) when compared with the analogous construct containing the full Tak1 N-terminus. This data suggests that the N-terminus has no direct inhibitory capacity for our proteins.

TABLE 2
Summary of data collection for TAK1-adenosine complex
Space Group: 1222
Unit Cell: a = 58.4 Å, b = 144.3 Å, c = 134.7 Å; α = β = γ = 90°

| Source | Daresbury SRS 14.2 |
|---|---|
| Wavelength (λ) | 1.488 |
| Resolution (Å) | 1.9 |
| No. of Reflections | 137639/38477 |
| (measured/unique) | |
| Completeness (%) | 85.2/48.8 |
| (overall/outer shell) | |
| I/σ(I) | 10.7/1.5 |
| (overall/outer shell) | |
| R_{merge} * (%) | 4.4/52.2 |
| (overall/outer shell) | |
| Molecules per asymmetric unit | 1 |

*R_{merge} = 100 × ΣₕΣⱼ |<I(h)> − I(h)ⱼ | / ΣₕΣⱼ <I(h)>

### Structure Refinement

| | |
|---|---|
| Resolution (Å) | 20-1.9 |
| No. of reflections | 35019 |
| R factor (%) | 21.2 |
| Free R factor (%) † | 23.1 |
| RMSD values | 0.008/1.6 |
| Bond lengths (Å)/angles (°) | |

† The Free R factor was calculated with 2.0% of the data.

TABLE 3
Summary of data collection for TAK1-3-
[6-(4-Acetyl-3,5-dimethyl-piperazin-
1-yl)-pyridin-2-yl]1H-pyrrolo[2,3-b]pyridine-
5-carboxylic acid methyl ester complex
Space Group: 1222
Unit Cell: a = 58.1 Å, b = 133.3 Å, c = 143.4 Å; α = β = γ = 90°

| Source | Vertex |
|---|---|
| Wavelength (λ) | 1.5438 |
| Resolution (Å) | 3.3 |
| No. of Reflections | 73870/8654 |
| (measured/unique) | |
| Completeness (%) | 99.2/98.6 |
| (overall/outer shell) | |
| I/σ(I) | 5.6/1.6 |
| (overall/outer shell) | |
| R_{merge}* (%) | 13.3/50.6 |
| (overall/outer shell) | |
| Molecules per asymmetric unit | 1 |

*R_{merge} =100 × ΣₕΣⱼ |<I(h)> − I(h)_{J} / ΣₕΣⱼ <I(h)>

### Structure Refinement

| | |
|---|---|
| Resolution (Å) | 20-3.3 |
| No. of reflections | 7852 |
| R factor (%) | 7.8 |
| Free R factor (%) †† | 32.2 |
| RMSD values | |
| Bond lengths (Å)/angles (°) | 0.019/1.6 |

†† The Free R factor was calculated with 2.5% of the data.

TABLE 4
Enzymatic characterization of proteins

| | Km (ATP) | Kcat |
|---|---|---|
| Protein | (μM) | (s-1) |
| M1-Q303: EFG₅: Q437-P504 | 24 ± 2 | 0.12 |
| I31-Q303: H468-P504 | 27 ± 1 | 0.26 |
| M1-Q303: H468-P504 | 21 ± 1 | 0.19 |

What is claimed is:

1. An isolated, purified human transforming growth factor-beta-activated kinase 1 (TAK1) construct of amino acids I31-Q303 that is directly fused to a human TAK1 binding protein (TAB1) segment of amino acids H468-P504.

2. A crystal comprising a TAK1 kinase domain of amino acids I31-Q303 that is directly fused to a TAB1 segment of amino acids H468-P504.

3. The crystal according to claim 2 wherein the TAK1 kinase domain is complexed with an active site inhibitor.

4. The crystal according to claim 3, wherein the active site inhibitor is an ATP, a nucleotide triphohsphate or an ATP analogue

5. The crystal according to claim 4, wherein the active site inhibitor is an ATP analog selected from the group consisting of adenosine, adenylyl imidodiphosphate, staurosporine and 3-(8-phenyl-5,6-dihydrothieno[2,3-h]quinazolin-2-ylamino)benzenesulfonamide.

6. The crystal according to claim 4, wherein the active site inhibitor is adenosine or 3-[6-(4- acetyl-3,5 - dimethyl-piperazine-1-yl)-pyridin-2yl]-1H-pyrrolo[2,3 -b] pyridine-5-carboxylic acid methyl ester.

7. The crystal according to claim 5 wherein the amino acids of TAK1 kinase domain and TAB 1 segment are coordinated according to the atom coordinates of FIG. 1.

8. The crystal according to claim 6 wherein the amino acids of TAK1 kinase domain and TAB1 segment are coordinated according to the atom coordinates of FIG. 2.

9. A crystallizable composition comprising:
a) TAK1 kinase domain; and
b) a TAB1 segment,
wherein the TAK1 kinase domain is directly fused to the TAB1 segment.

10. The crystallizable composition according to claim 9 further comprising 600-900 mM sodium citrate, 1 to 200 mM sodium chloride, and a buffer that maintains pH at between about 6.5 and about 8.5 .

11. The crystallizable composition according to claim 10 further comprising a reducing agent at between about 1 to about 20 mM.

12. The crystallizable composition according to any one of claims 9-11, wherein the TAK1 kinase domain comprises residues I31-Q303 of human TAK1.

13. The crystallizable composition according to any one of claims 9-11, wherein the TAK1 kinase domain comprises residues I31-Q303 of human TAK1 and the TAB1 segment comprises amino acids H468-P504 of human TABl.

14. A method of utilizing molecular replacement to obtain structural information about a molecule or a molecular complex of unknown structure, wherein the molecule is sufficiently homologous to TAK1, comprising the steps of:
(a) crystallizing said molecule or molecular complex;
(b) generating an X-ray diffraction pattern from said crystallized molecule or molecular complex; and
(c) applying at least a portion of the structure coordinates of FIG. 1 or FIG. 2 to the X-ray diffraction pattern to generate a three-dimensional electron density map of at least a portion of the molecule or molecular complex of unknown structure; and
(d) generating a structural model of the molecule or molecular complex from the three-dimensional electron density map.

15. A method of identifying a TAK1 binding compound, comprising the step of using at least a portion of the structure coordinates of FIG. 1 or FIG. 2 to computationally screen a candidate compound for an ability to bind an ATP-binding site of TAK1.
